SEQUENCE LISTING (41 sequences)

SEQ ID NO: 1 - protein, 9 aa, synthetic construct
  REGION 1..9: note = HERV epitope
KLLGDINWI

SEQ ID NO: 2 - protein, 9 aa, synthetic construct
  REGION 1..9: note = HERV epitope
FIFTLIAVI

SEQ ID NO: 3 - protein, 9 aa, synthetic construct
  REGION 1..9: note = HERV epitope
RMLTDLRAV

SEQ ID NO: 4 - protein, 9 aa, synthetic construct
  REGION 1..9: note = HERV epitope
FLSLYFVSV

SEQ ID NO: 5 - protein, 9 aa, synthetic construct
  REGION 1..9: note = HERV epitope
TLIAVIMGL

SEQ ID NO: 6 - protein, 9 aa, synthetic construct
  REGION 1..9: note = HERV epitope
YLDQIATLI

SEQ ID NO: 7 - protein, 9 aa, synthetic construct
  REGION 1..9: note = HERV epitope
FLQAEVANA

SEQ ID NO: 8 - protein, 9 aa, synthetic construct
  REGION 1..9: note = HERV epitope
WMGDRLMSL

SEQ ID NO: 9 - protein, 9 aa, synthetic construct
  REGION 1..9: note = HERV epitope
ALHSSVQSV

SEQ ID NO: 10 - protein, 9 aa, synthetic construct
  REGION 1..9: note = HERV epitope
ILTEVLKGV

SEQ ID NO: 11 - protein, 9 aa, synthetic construct
  REGION 1..9: note = HERV epitope
LMAQAITGV

SEQ ID NO: 12 - protein, 9 aa, synthetic construct
  REGION 1..9: note = HERV epitope
RLSYVHVTV

SEQ ID NO: 13 - protein, 9 aa, synthetic construct
  REGION 1..9: note = HERV epitope
VLQDFDQPI

SEQ ID NO: 14 - protein, 9 aa, synthetic construct
  REGION 1..9: note = HERV epitope
ALMIVSMVV

SEQ ID NO: 15 - protein, 9 aa, synthetic construct
  REGION 1..9: note = HERV epitope
MLLAALMIV

SEQ ID NO: 16 - protein, 9 aa, synthetic construct
  REGION 1..9: note = HERV epitope
YIDDILCAA

SEQ ID NO: 17 - protein, 9 aa, synthetic construct
  REGION 1..9: note = HERV epitope
YIIHYIDDI

SEQ ID NO: 18 - protein, 9 aa, synthetic construct
  REGION 1..9: note = HERV epitope
YIWCPTWRL

SEQ ID NO: 19 - protein, 9 aa, synthetic construct
  REGION 1..9: note = HERV epitope
YIWCPTWSL

SEQ ID NO: 20 - protein, 666 aa, Homo sapiens
  REGION 1..666: note = HERV-K10 Gag protein
MGQTKSKIKSKYASYLSFIKILLKRGGVKVSTKNLIKLFQIIEQFCPWFPEQGTSDLKDWKRIGKELKQAGRKGNIIPLTVWNDWAIIKAALEPFQTEEDSISVSDAPGSCLIDCNENTRKKSQKETESLHCEYVAEPVMAQSTQNVDYNQLQEVIYPETLKLEGKGPELMGPSESKPRGTSPLPAGQVLVRLQPQKQVKENKTQPQVAYQYWPLAELQYRPPPESQYGYPGMPPAPQGRAPYHQPPTRRLNPMAPPSRQGSELHEIIDKSRKEGDTEAWQFPVTLEPMPPGEGAQEGEPPTVEARYKSFSIKMLKDMKEGVKQYGPNSPYMRTLLDSIAYGHRLIPYDWEILAKSSLSPSQFLQFKTWWIDGVQEQVRRNRAANPPVNIDADQLLGIGQNWSTISQQALMQNEAIEQVRAICLRAWEKIQDPGSTCPSFNTVRQGSKEPYPDFVARLQDVAQKSIADEKAGKVIVELMAYENANPECQSAIKPLKGKVPAGSDVISEYVKACDGIGGAMHKAMLMAQAITGVVLGGQVRTFGGKCYNCGQIGHLKKNCPVLNKQNITIQATTTGREPPDLCPRCKKGKHWASQCRSKFDKNGQPLSGNEQRGQPQAPQQTGAFPIQPFVPQGFQGQQPPLSQVFQGISQLPQYNNCPSPQAAVQQ

SEQ ID NO: 21 - protein, 666 aa, Homo sapiens
  REGION 1..666: note = HERV-K113 Gag protein
MGQTKSKIKSKYASYLSFIKILLKRGGVKVSTKNLIKLFQIIEQFCPWFPEQGTLDLKDWKRIGKELKQAGRKGNIIPLTVWNDWAIIKAALEPFQTEEDSVSVSDAPGSCIIDCNEKTRKKSQKETESLHCEYVAEPVMAQSTQNADYNQLQEVIYPETLKLEGKGPELMGPSESKPRGTSPLPAGQVPVTLQPQKQVKENKTQPPVAYQYWPPAELQYQPPPESQYGYPGMPPAPQGRAPYPQPPTRRLNPTAPPSRQGSELHEIIDKSRKEGDTEAWQFPVTLELMPPGEGAQEGEPPTVEARYKSFSIKMLKDMKEGVKQYGPNSPYMRTLLDSIAHGHRLIPYDWEILAKSSLSPSQFLQFKTWWIDGVQEQVRRNRAANPPVNIDADQLLGIGQNWSTISQQALMQNEAIEQVRAICLRAWEKIQDPGSTCPSFNTVRQGSKEPYPDFVARLQDVAQKSIADEKARKVIVELMAYENANPECQSAIKPLKGKVPAGSDVISEYVKACDGMGGAMHKAMLMAQAITGVVLGGQVRTFGGKCYNCGQIGHLKKNCPVLNKQNITIQATTTGREPPDLCPRCKKGKHWASQCRSKFDKNGQPLSGNEQRGQPQAPQQTGAFPIQPFVPQGFQGQQPPLSQVFQGISQLPQYNNCPPPQAAVQQ

SEQ ID NO: 22 - protein, 665 aa, Homo sapiens
  REGION 1..665: note = HERV-K21 Gag protein
MGQTKSKIKSKYASYLSFIKILLKRGGVKVSTKNLIKLFQIIEQFCPWFPEQGTLDLKDWKRIGKELKQAGRKGNIIPLTVWNDWAIIKAALEPFQTEEDSISVSDAPGSCIIDCNENTRKKSQKETEGLHCEYAAEPVMAQSTQNVDYNQLQEVIYPETLKLEGKGPELVGPSESKPRGTSPLPAGQVPVTLQPQTQVKENKTQPPVAYQYWPPAELQYRPPPESQYGYPGMPPAPQGRAPYPQPPTRRLNPTAPPSRQGSELHEIIDKSKEGDTEAWQFPVMLEPMPPGEGAQEGEPPTVEARYKSFSIKMLKDMKEGVKQYGPNSPYMRTLLDSIAHGHRLIPYDWEILAKSSLLPSQFLQFKTWWIDGVQEQVQRNRAANPPVNIDADQLLGIGQNWSTISQQALMQNEAIEQVRAICLRAWEKIQDPGSTCPSFNTVRQSSKEPYPDFVARLQDVAQKSIADEKARKVIVELMAYENANPECQSAIKPLKGKVPAGSDVISEYVKACDGIGGAMHKAMLMAQAITGVVLGGQVRTFGGKCYNCGQIGHLKKNCPVLNKQNITIQATTTGREPPDLCPRCKKGKHWASQCRSKFDKNGQPLSGNEQRGQPQAPQQTGAFPIQPFVPQGFQGQQPPLSQVFQGISQLPQYNNCPPPQAAVQQ

SEQ ID NO: 23 - protein, 956 aa, Homo sapiens
  REGION 1..956: note = HERV-K113 Pol protein
NKSRKRRNRVSFLGAATVEPPKPIPLTWKTEKPVWVNQWPLPKQKLEALHLLANEQLEKGHIEPSFSPWNSPVFVIQKKSGKWRMLTDLRAVNAVIQPMGPLQPGLPSPAMIPKDWPLIIIDLKDCFFTIPLAEQDCEKFAFTIPAINNKEPATRFQWKVLPQGMLNSPTICQTFVGRALQPVRDKFSDCYIIHYIDDILCAAETKDKLIDCYTFLQAEVANAGLAIASDKIQTSTPFHYLGMQIENRKIKPQKIEIRKDTLKTLNDFQKLLGDINWIRPTLGIPTYVMSNLFSILRGDSDLNSKRMLTPETTKEIKLVEEKIQSAQINRIDPLAPLRLLIFATAHSPIGIIIQNTDLVEWSFLPHSTVKTFTLYLDQIATLIGQTRLRIIKLCGNDPDKIVVPLTKEQVRQAFINSGAWQIGLANFVGIIDNHYPKTKIFQFLKLTTWILPKITRREPLENALTVFTDGSSNGKAAYTGLKERVIKTPYQSAQRAELVAVITVLQDFDQPINIISDSAYVVQATRDVETALIKYSMDDQLNQLFNLLQQTVRKRNFPFYITHIRAHTNLPGPLTKANEQADLLVSSALIKAQELHALTHVNAAGLKNKFDVTWKQAKDIVQHCTQCQVLHLPTQEAGVNPRGLCPNALWQMDVTHVPSFGRLSYVHVTVDTYSHFIWATCQTGESTSHVKKHLLSCFAVMGVPEKIKTDNGPGYCSKAFQKFLSQWKISHTTGIPYNSQGQAIVERTNRTLKTQLVKQKEGGDSKECTTPQMQLNLAPYTLNFLNIYRNQTTTSAEQHLTGKKNSPHEGKLIWWKDNKNKTWEIGKVITWGRGFACVSPGENQLPVWMPTRHLKFYNEPIGDAKKSTSAETETPQSSTVDSQDEQNGDVRRTDEVAIHQEGRAADLGTTKEADAVSYKISREHKGDTNPREYAACSLDDCINGGKSPYACRSSCS

SEQ ID NO: 24 - protein, 1117 aa, Homo sapiens
  REGION 1..1117: note = HERV-K9 Pol protein
MGQTKSKIKSKYASYLSFIKILLKRGGVKVSTKNLIKLFQIIEQFCPWFPEQGTLDLKDWKRIGKELKQAGRKGNIIPLTVWNDWAIIKAALEPFQTEEDSISVSDAPGSGIIDCNEKTRKKSQKETESLHCEYVAEPVMAQSTQNVDYNQLQEVIYPETLKLEGKGPELVGPSESKPRGTSPLPAGQVPVTLQPQKQVKENKTQPPVAYQYWPPAELQYRPPPESQYGYPGMPPAPQGRAPYPQPPTRRLNPTAPPSRQGSELHEIIDKSRKEGDTEAWQFPVTLEPMPPGEGAQEGEPPTVEARYKSFSIKILKDMKEGVKQYGPNSPYMRTLLDSIAHGHRLIPYDWEILAKSSLSPSQFLQFKTWWIDGVQEQVRRNRAANPPVNIDADQLLGIGQNWSTISQQALMQNEAIEQVRAICLRAWEKIQDPGSTCPSFNTVRQGSKEPYPDFVARLQDVAQKSIADEKARKVIVELMAYENANPECQSAIKPLKGKVPAGSDVISEYVKACDGIGGAMHKAMLMAQAITGVVLGGQVRTFGGKCYNCGQIGHLKKNCPVLNKQNITIQATTTGREPPDLCPRCKKGKHWASQCRSKFDKNGQPLSGNEQRGQPQAPQQTGAFPIQPFVPQGFQGQQPPLSQVFQGISQLPQYNNCPPPQVAVQQVDLCTIQAVSLLPGEPPQKIPTGVYGPLPEGTVGLILGRSSLNLKGVQIHTSVVDSDYKGEIQLVISSSVPWSASPGDRIAQLLLLPYIKGGNSEIKRIGGLGSTDPTGKAAYWASQVSENRPVCKAIIQGKQFEGLVDTGADVSIIALNQWPKNWPKQKAVTGLVGIGTASEVYQSMEILHCLGPDNQESTVQPMITSIPLNLWGRDLLQQWGAEITMPAPLYSPTSQKIMTKRGYIPGKGLGKNEDGIKIPFEAKINQKREGIGYPFLGAATIEPPKPIPLTWKTEKPVWVNQWPLPKQKLEALHLLANEQLEKGHIEPSFSPWNSPVFVIQKKSGKWRMLTDLRAVNAVIQPMGPLQPGLPSPAMIPKDWPLIIIDLKDCFFTIPLAEQDCEKFAFTIPAINNKEPATRFQWKVLPQGMLNSPTICQTFVGRALQPVKVFRLLYYSLY

SEQ ID NO: 25 - protein, 956 aa, Homo sapiens
  REGION 1..956: note = HERV-K6 Pol protein
NKSRKRRNRESLLGAATVEPPKPIPLTWKTEKPVWVNQWPLPKQKLEALHLLANEQLEKGHIEPSFSPWNSPVFVIQKKSGKWRMLTDLRAVNAVIQPMGPLQPGLPSPAMIPKDWPLIIIDLKDCFFTIPLAEQDCEKFAFTIPAINNKEPATRFQWKVLPQGMLNSPTICQTFVGRALQPVREKFSDCYIIHCIDDILCAAETKDKLIDCYTFLQAEVANAGLAIASDKIQTSTPFHYLGMQIENRKIKPQKIEIRKDTLKTLNDFQKLLGDINWIRPTLGIPTYAMSNLFSILRGDSDLNSKRMLTPEATKEIKLVEEKIQSAQINRIDPLAPLQLLIFATAHSPTGIIIQNTDLVEWSFLPHSTVKTFTLYLDQIATLIGQTRLRIIKLCGNDPDKIVVPLTKEQVRQAFINSGAWKIGLANFVGIIDNHYPKTKIFQFLKLTTWILPKITRREPLENALTVFTDGSSNGKAAYTGPKERVIKTPYQSAQRAELVAVITVLQDFDQPINIISDSAYVVQATRDVETALIKYSMDDQLNQLFNLLQQTVRKRNFPFYITHIRAHTNLPGPLTKANEQADLLVSSALIKAQELHALTHVNAAGLKNKFDVTWKQAKDIVQHCTQCQVLHLPTQEAGVNPRGLCPNALWQMDVTHVPSFGRLSYVHVTVDTYSHFIWATCQTGESTSHVKKHLLSCFAVMGVPEKIKTDNGPGYCSKAFQKFLSQWKISHTTGIPYNSQGQAIVERTNRTLKTQLVKQKEGGDSKECTTPQMQLNLALYTLNFLNIYRNQTTTSAEQHLTGKKNSPHEGKLIWWKDNKNKTWEIGKVITWGRGFACVSPGENQLPVWIPTRHLKFYNEPIRDAKKSTSAETETSQSSTVDSQDEQNGDVRRTDEVAIHQEGRAANLGTTKEADAVSYKISREHKGDTNPREYAACSLDDCINGGKSPYACRSSCS

SEQ ID NO: 26 - protein, 699 aa, Homo sapiens
  REGION 1..699: note = HERV-K113 Env protein
MNPSEMQRKAPPRRRRHRNRAPLTHKMNKMVTSEEQMKLPSTKKAEPPTWAQLKKLTQLATKYLENTKVTQTPESMLLAALMIVSMVVSLPMPAGAAAANYTYWAYVPFPPLIRAVTWMDNPIEIYVNDSVWVPGPTDDCCPAKPEEEGMMINISIGYRYPPICLGRAPGCLMPAVQNWLVEVPTVSPISRFTYHMVSGMSLRPRVNYLQDFSYQRSLKFRPKGKPCPKEIPKESKNTEVLVWEECVANSAVILQNNEFGTLIDWAPRGQFYHNCSGQTQSCPSAQVSPAVDSDLTESLDKHKHKKLQSFYPWEWGEKGISTARPKIISPVSGPEHPELWRLTVASHHIRIWSGNQTLETRDRKPFYTIDLNSSLTVPLQSCVKPPYMLVVGNIVIKPDSQTITCENCRLLTCIDSTFNWQHRILLVRAREGVWIPVSMDRPWEASPSVHILTEVLKGVLNRSKRFIFTLIAVIMGLIAVTATAAVAGVALHSSVQSVNFVNDWQNNSTRLWNSQSSIDQKLANQINDLRQTVIWMGDRLMSLEHRFQLQCDWNTSDFCITPQIYNESEHHWDMVRCHLQGREDNLTLDISKLKEQIFEASKAHLNLVPGTEAIAGVADGLANLNTVTWVKTIGSTTIINLILILVCLFCLLLVYRCTQQLRRDSDHRERAMMTMVVLSKRKGGNVGKSKRDQIVTVSV

SEQ ID NO: 27 - protein, 9 aa, synthetic construct
  REGION 1..9: note = Peptide 1
FLQFKTWWI

SEQ ID NO: 28 - protein, 9 aa, synthetic construct
  REGION 1..9: note = Peptide 2
RLIPYDWEI

SEQ ID NO: 29 - protein, 9 aa, synthetic construct
  REGION 1..9: note = Peptide 3
KLIDCYTFL

SEQ ID NO: 30 - protein, 9 aa, synthetic construct
  REGION 1..9: note = Peptide 4
YLSFIKILL

SEQ ID NO: 31 - protein, 9 aa, synthetic construct
  REGION 1..9: note = Peptide 5
AMIPKDWPL

SEQ ID NO: 32 - protein, 9 aa, synthetic construct
  REGION 1..9: note = Peptide 6
YAMSNLFSI

SEQ ID NO: 33 - DNA, 7557 bp, synthetic construct
  source 1..7557: mol_type = other DNA
  misc_feature 1..7557: note = HERV
tggccaattcctgccttaactgatgacattgtcttgtgaagttccttctcctggctcatcctggctcaaaagctcccctactgagcaccttgtgacccccactctgcctgccagagaacaacccccctttgactgtaattttcctttacatacccaaatcctataaaacggccccaccccatctcccttcgctgactcttttcggactcaacccacctgcacccaggtgaaataaacagctttattgctcacacaaagcctgtttggtggtctcttcacacagacgcacatgaaatttggtgctgtgactcggatcgggggacctcccttgggagatcaatccccggtcctcctgttctttgctccatgagaaagatccacctgtgacctcaggtcctcagaccgaccagcccaagaaacatctcaccaatttcaaatccagtaagcggcctctttttactctcttctccaacctccctcactatccctcaacctctttctcctttcagccttggcaccacacttcaatctctcccttcttttaatttcaattcctttcattttctggtagagacaaaggagacacattttatccgtggacccaaaactccggcaccggtcacggactgggaaggcagccttcccttggtgtttaatcattgcagggacacctctctgattattcacccatgtttcagaggtgtcagaccacgcagggatgcctgccttggtccttcatgcttagcggcaagtcccacttttctgggggaggggcaagtaccccaaccccttctgtgtctctaccccttctccgcctttctggggggcaagaaacccccgaccacttctccttcacccttagcggcaagtcccacttttctaggggaggagcaagtaccccaacctcgtatctctgcgccccgatctcttatttccgcaccctgaccccttatatctctgcaccctgatcccttatttccatgccccaacctcttatatctctgcacccgatcccttatttccacaccccaacctctatatctctgtgccccaatcccttatttccacgccctgacctcgtatctctgtgccccgacccctttcccgcttttctggagggtaagaacccccaaaccccttccctccatgtctctactctcttttctctgggcttgcctccttcactatgggcaaacttccaccctccattcctacttcttctcccttagcctgtgttcttaagaacttaaaacctcttcaactctcacctgacctaaaatctaagcatcttattttcttctgcaatgccgcttgaccccaatacaaactcgacagtagttccaaatagccagaaaatggcactttcaatttttccatcctgcaagatctaaataattcttgtcataaaataggcaaacggtctgaggtggctgacgtccaggcattcttttacacatcagtccctttctagtctctgtgcccagtgcaactcgtcccaaatccttctttccctcccacttgtcccctcagtcccaaccccaagcgtcactgagtctttctaatcttccttttctacagatctatctgacctctcccctcctggccaggctgaggtaggtcccaattcttcctcagcctccgctcctccaccctataatccttttatcacctcccctcctcacacccagtccggcttacagtttcgttccgtgtctagtcctcccccacctgcccagcaattcactgttaaaaaggtggctgaagctaaaggcatagtcaaggttaatgctcctttttatttatcagacctctcccaaatcagtgagcatttagactctttcatcaaatatgaaaaacccagcccagttcatgactcgtttggcagcaaccctgagacactttacagccctagaccctaaaaggtcaaaaggccgtcttattctcaaaatacattttattacccaatctgctcttgacattaaataaaactccaaaaattaaattccagccctcaaaccacacaacaggatttaattaacctagccttcaaggtgtacaataatagaaaaaagttgcaattccttgcctccactgtgagacaaaccccagacacatctccagcacacaagaacttccaaacgcttgaaccgcagccaccaggcgttcctccagaagctcctcccccaggagcttgctacaagtgccagaaatctggccaccaggccaaggaatgcctgcagcccaggattcctcctaagccgcgtcccatctgtgcgggaccccactggaaatcggactgtcccactcaccttggcagccactcccagagcccctggaactctggcccaaggctctctgactccttcccagaccttcttggcttagcagctgaagactgaagctgcccgatcgcctcagaatccccctagaccatcacggacgccaagcttcgggtaactctcacagtggaaggtaagtccgtccccttcttaatcaatacggaggctacccactccacattaccttattttcaagggcctgtttcccttgcctccataactgttgtgggtattgatggccaggcttctaaacctcttaaaactccccaactctggtgccaacttagacaatactcttttaagcgctccttttagttatccccacctgcccagttcccttattaggccgagacactttaactaaattatctgcttccctgactattcctggactacagctacatctcattgctgccctcttcccaatccaaagcctcctttgcatcctcctcttgtattcccccaccttaacccacaagtataagatacctctactccctccttggcgaccgatcatgcaccccttaccatcccattaaaacctaatcacccttacccggctcaatgccaatatcccatcccacagcatgctttgaaaggattaaagcctgctatcactcacctgctacagcatggccttttaaagcctataaactctccttataatttccccattttaccggtcctaaaaccagacaagccttacaagttagttcaggatctgcgccttatcaaccaaattgttttgcctatccaccccgtggtgccaaacccatatactctcctatcctcaatacctccctccacaatccattattctgttctggatctcaaacatgctttctttactattcctttgcacccttaatcccagcctctcttcgctttcacttggactgaccctgacacccatcaagctcagcaaattacctaggctgtactgccgcaaagcttcacagacagcccccattacttcagtcaagcccaaatttcttccttatctgttacctatctcagcataattctcataaaaacacacgcgctgtccctgctgatcgtgtccgattaatctcccaaacctcaatcccttacaaaacaacaactcctttccgtcctaggcatggttagtgcggtcagagatcttacacaagagccaggaaggcaccctgtagcctttctgtccaaacaacttgaccctactgttttaggctggccaccatgtctccttgcagcggctgctgccgccctaatacttttagaggccctcaaaatcacaaactatgctcaacttactctctacatttctcataacttccaaaatctattttcttcctcatacctgatgcatatactttctgtaccccggctccttcagctgtacttactctttgttaagtcccgcaattaccattgttcctggcccggacttcaatccggcctcccacattattcctgataccacacctgaccctcatgactgcatctctctgatccacctgcgttcaccccatttccccacatttcctccttccctgtctctcaccctgatcacacttagtttattgatggcggttccaccaggcctaatctccacacaccagcaaaggcaggctatgctacagtaccagccactagcccgcctcttagaacctctcatttcctttccatcgtggaaatctatcctcaaggaaataacttctcagtgttccatctgctattctactactcctcagggattattcaggccccctcccttccctacacatcaagctcgaggatttgcccccacccaggactggcaaattagctttactcaacatgccccgagtcagataactaaaatatctcttagtctaggtagacactttcactatataggtagagtcctttcctacagggtctgagaaggccaccgcagtcatttcttcccttctgtcagacataattcctcagtttagccttcccacctctatacagtctgataacagaccagcctttattagtcaaatcagccaagcagtttttcaagctcttagtattcagtgaaacctttatatcccttacggtcctcagtcttcaggaaaagtagaacagactaatggtcttttaaaaacacacctcaccaagctcagccaccaacttaaaaaggactagacaatacttttaccactttcccttctcagaagtcagacctgtcctcagaatgctacagcgtacagcccatttgagctcctgtatggatgctcctctttattaggccccagtctcattccagacaccagactaacttagactgtgccccagaaaaacttgtcatccctaccatcttctgtctagtcacactcctattcaccgttctcagctactcatacatgccctgctcttgtttacactgccggtttacactgtttctccaagtcatcacagctgatatcccctggtgctatccccaaactgccactcttagctcttgaagtaaataaataatctttactggcaaggctatgctgaacctccttaggcactctctaattagatgtcccgggtcctcccaattcttagacctttaatacctgtttttctccttctcttattccgtttagtttttcaattcatacaaaaccgtatccaggccatcaccaataattctaaatgaaaaatgtttcttctaacaaccccacaatatcaccccttaccacaaaatcttccttcagcttaatctctcccactgtaggttcccacgccgcccctaatcccgctcaaagcagccctgagaaacatcacccattatctctccatatcacccccaaaaattttcgacatcccaacactttaccactatttcgttttatttttcttattaatataagaagacaggaatgtcaggcctctgagcccaagctaagccatcatatcccctgtgacctgcacgtgcacatccagatggccggttcctgccttaactgatgacattccaccataaaagaagtgaaaatggcctgttcctgccttaactgatgacatcgtcttgtgaaattccttcttctggctcatcctggctcaaaagctcccctactgagcaccttgtgacccccactctgcccgccagagaacaacccccctttgaatgtaattttcctttacgtacccaaattctataaaacggccccaccccatctcccttcgctgactctcttttcggactcaactcacctgcacccaggtgaaataaacagctttattgctcacacaaagcctgtttggtggtctcttcacacagacgcgcatgaaacatatcaactaaatgcacagggctaatgttgaaggcgctctgaggattggatggtagcaatgtatcaatgttaattttctgaaggctatactatggttaggaaggacagtgttcttgtttgtaaaaaataccattcaaaggtgataggacattagatcagaaacttactcaggaaaagaacagtccttttgctgtgcttccaactgttttataagtttgtggctatttcaaaatttnnnnnnnnnnnngaaagaaagcaactgttccaagcaatgtaactatcttcaagtacaatgtcaaatttgtacatgctagctaaggtaatcaattccctgctccccccaagaaaacacagtgaggtattttcagttgctgaatatcaaatgtttattctttgccttcttactactgttaaaatctctttgataagtggctcctcttaaaactggatttgatttcacagaaattttgtgaattattattcatcttgaggtgtcccatgctggggatgaggatgtgtcgaggcacagcttggacaaccaggccaggtgggccaccatgaaggttctcctccccataccccaccccacacacagaaacctgcaaacaactcagggaacacctgtgcaaagccagtcaggttgtccaagtcagctgaaaacagaacaccatactagattcaaattacaagttcagtactgtactgtacacatccagcattacacatgcttaagataatataagtatgcataatgacatatcgggtctcacttgtaaactataagcacacagttacaaaatgttaaactacaacataatcccagtttacctaaagttacaaatgttgttgcatcgacattggctgcttcagaagccttgggcatgcagacaggaagtagagaaaatattccagctaagaataatcatattccacatatttaagaatctttctagccaaatcatattctaaagaaggctatcttgaagggccagggccaatttccaggctgtgtggtttttatgagagggaattgtgccctgacacatatgtgctcttcacagatcctggaattatgaatgcaggtagaccactgtgattcccagggtgacttcagcctctgggagcaagaatacctagtggcctctggctcaggggagtctctcaaccctggaattccacccaggtctggagggctgctccaggctgatggggaccattttgacctgtcaagcccagaggcaccatgggtatagtctgacagcaacctatagtaactgcagctttcaggctaaatgaatattctgctctaccccatgagaatgctggcccattaaagatgattttgtttgcaattttaatatgatctacaaaaagaactaaacttgtaattaatagcaccatgaaaacatagataatggtagaaaataggataataaaataaatttgctttcaattccgggcaatgtttcctaccactaacttgaacaaagtaaaccattttaactgaatttgagaaaagaacatgtctttctggagcaaaaatattcttgtagggagaatataggcaaatatattcattatgaattttatatacatatgtgtgtatatatgtatgcatagatatataaaactattgagcagcagaggaaagaatgtaagacatagctctctattgagagtggctgttgggctgtcaagcagtcatgtagaagtatttttaaaaagaaaattgagggcacaagaaagcatctaaagtcttggtaaaatggtcaactggggttcagcaattgaaggagagtactgagtatgtactaatgtacagttcacatcaacatctcagaaataaaatagctccagctttgaagcatctcccttgactgattaaaagaggaattctcatccctacagaaagaagaggtagtatg

SEQ ID NO: 34 - DNA, 7594 bp, synthetic construct
  source 1..7594: mol_type = other DNA
  misc_feature 1..7594: note = HERV
tggttattttatctatagattccagacattgcatagacaagcactgtgaaaatcccctgtgctgttgtgctccattctgattaccggtgtatgcagctcccagtcatataccccactgcttgctcaatcaatcacgaccctttcacgcagactcccttggagttgtaagcccttaaaagggacaggaattgcttactcggggagctcggtttttggagacgtgagtctgccaatgctcctagctgaataaagcccttccttctacaactcagtgtctgaggggttttgtctgtgtcttgtcctactacattgcttggtattctgaccagaaagcgaggtgattaacagacatggtctaggcagccccttaggcagcttaggcctgctctgtggagcatccctgcaggggactgcagccagcttgagcaacatggatcctgagagcactcctgggtaggcattttccctggtggaatgccttgttagagcagagcatggcagaccccaatggaggatcaatatggtggctgaacaccaggaggaactggcacttggagtccggacatctgaaacttggtaagactggtctttagaccttgcccactccattttagtggaagcatggcctgatcacccacagcgtgcctgtaccggcactttggtttttgtttttgacttgacttggattgcttgatacttcggttttggttttgacctggcttggatttcttgatactctgattttggttttgattctggtttggtgtaaactgtaaaagtgcgtgtgtgccctttttatccgttctttgttttgtggtgtgcatgaggtgtgagtgtggtgttttgtctcaaggaagcatgggtcaggcacaaagtaagcccaccacactaggaaatatgtggaaaaatttcaagaaaagatttaagggagactatggagtactatgacaccaggaaaacttaaaactttgtgtaaggtagactggccagcattagaggtggccatcagaaggaagcctggacaggtcccttgtttcaaaggtatggcacaaggtaacttgtaagccagggcacccagaccagttcccatacatagacacttggttacagctggttttagaccaccccccacagtggttgagagaacagcagcataagcggctggcagaggcaaggaaagaccagcagagagagagaaaggaagagacagagagacaaagagggagtcaagaagagagagagagaaagagaagcagagagagaggaagagacagaggcaaaaggaaagtcaaagagagagagagagacagaaagtcaaagagagagacagagaaatatacaagtagttaagaaaaaaaaagtgtacactattcctttaaaagccaaggtaaatttaaaacctataattgataattaaaggtattctccataagcctataacactccaataccactttgttgtcagtgtaaacaagggggtatcccaaaagcactgaggccttcctatcaaaaatccttagccctgtaacctatggatggcccaaatgcattcaatctgtagtggcaactgctttgctaacagaagaaagtaaaaaataacttttagaggaaacctcattgtgagcacacctcaccagttcagaagtatcctaaggnnnnnnnnnnnnnnnnnnnnnnnnnnncgatgatttaacattaaccactgaaaattcccttaacccagcaggtttcccaacaggggatctaaatcttaattaccatacaaaggtccgaccagacctaggaggaactcccttcaggacaggatgatagatggttcctcccgggcaattgaaggnnnnnnnnnnnngccatctataccaattctaagttagtttggactaaacaaggtcttattaatagcaaaggataattgaaatcccaaacttacaaggttttcaacaaaagtaaagtttactaaaagttaacagtgtaacatgtattatagtaacctgtaatcttgtggccttagacagtctagtccacagacataaaggaagtttgctttggaaaagaacggttatcttcaggaaaaaaaggggggcagaatttatgcaaaaataatgttatatggtaaattcttgtcctgaaataaattaaccggttgtttaaagaaagaaatgtttgtaataagtcagacagctgaggcatgttgaagaattgtctgtgaaagtcgtgaaagagaaaaaaagttataaaaaaaagtgtgttagaaaaagaatttatgcaagaaatgttgtataatttaaaagtaattaggcctcctgaatgtaaaactattgaagagacagttttgtgcaaggtgtataaggaaagtaaagtatacctttggtaaagggattataaggagacataagaatgtggactttttacctacattaaagggttaaaaaaatgttttaaaggtttaagcaagttttcaaatgttagttgtgtgtaaattctgtgtgtaaatatattagctaaagttaaaggggtatcatccagtttttctgagaactggacattaaagtaaaaacacaacaggtttctcttaaagcactaacctgctctttaacaaagattataaaaggttaaaaagagtctataaaaatcttacctaatggtccgacattaaaaattgaataaatatgcctacaaagttttattaaaactaactttaacattaatagcatactaatataaaggtgaaatttagcttatctggtataaaaatcatacaagaagaattgtcaaatataaaatagtgtttggcttctttggtctaaaaactaataaaaaataggtactaaaggaaatttcgtagtagaaaggcaccaaggactataaagtccactgccgatgtccccacatttaaaacaaaaggtcaatttctttaaaaaataattacatacttgctttatcttccactttcctttccctcaaaactaaaagtcttttagcacatgtaccacccctagaatttctggtaaacccgcaccagcctgaagatcatgttctcatcaaagggtggaaagaaaaaaactcgagccagcctgagaaggaccctaccttgtgctgctatccaccgagactgctgttcatacagcagaaaaggaatggactcatcacacccaagccaagaaagcaccaccccctccagagttgtgggccacagtcccaggagaaaaccctacaaaactaaagctaagaaaagtttaactccttcttctattctatcgctctttcttctttcttcactctatttctgaccatctagttattaacataaccaagtcaattttgcctcaaactattgcatttaatgcttgccttgttataccctgtggggacttgccaagtcaaagacagctctctacttcagaaaagtacctctgtccctcctgactctcctcagactgggcattaataaattgggaccatttaatccagggagattttgattaaaaaccccagtgtcaatcaggagtcttgccccccagtagagagtttttatgctgtagttggtccaatgttctgtggaccactaaagagcaaggatggactgccccaaccggtttttgtaatttcctaaaatcatacattcattttactagaggatcatagacgttaaagactgaaaacaaactttggcaattaagcaggatgccaagatgcaaatgcctggttgaaatggatcaaatattctgtccacacataaaacaaaagcaattgttatgcttgtgcacatggcagtccagaggcccagattgtcccctttccactggggtggtcctccagtcgaccaggcatgggctgcatggtagctcttttccaggattctacagcctggagtaatcagtcatgccaagctccctctgctatatcccaaagtccagcaccctgtgggtcagcccccaggggacatccagcttccatctcccaacactaagttcacttcttgtctctcacgacagggaggaaacttagccttctttggagacctgaagggatgcaatgagcttaagaattttcaagagcttatcaatcagtcagcccttgttcatccctgagcagatgtgtggtggtattgtggtggacctttactaggcactgtgcccaataactggagtggtacttgtactttagtccagttggctatccctttcaccctggcatttcatcaaccagagggaggaaaaataagatatcataaagcaagagaagccccttatgggtctttcaactctcacgtctatttagatgcaattgaagtcccatgaggaataccagatcaatttaaagcccaaaatcaaatagctgcaggatttgagtcaatatttcggtgggtgacagttaataaaaatgtagattggataaactacatctgttaaaaccaacagcaacaagcttttcatgagttaaaagaaaaactcatgtcggccccagccctggggctacatgacctgacaaaatctttaacactatatgtgtcagagagaaaaaaaagtggcagatggagttttgacccagactgtggggccctggctgaggcctgcctacctcgtcaaagatctagatggagtttctaagagttggcccctgtgcttaagagccttggcagcaacagctctgctagcacaagaggtggataagctaactcttgggcaaaacctaaacttaaaggccccccacgctatggtgactttcatgaatacatcattggctaacaaatgctagattaaccaagtaccaaagcttgctatgtgaaaatccccgcataaccattgagttttgcaacaccctaaaccctgccaccttgctcccagtatcagaaagcccagttgaacataactgtgtagaggtattggactccgtttattctagtgggaccaatctccaagaccacccttgaacagtagactgtgagcggtacatgaatgggagcagcttcgccaacccctgtaaagtgactctgaagaagacgagaagccctgctccagtcacacccagaagctaactggtccacgcatggctgaagcatgagaaaactcttcatgggactcattttccttaaaatttggacttgtatagtaagtacttcaactgaccttcctcagactgaggactgttcccagtgtatatatcaagtcacagaggtaggacaaaaagttgctacagtcttattttatgcttattgtaagtgtactgggactgtaaaaagaacttgtttgtataatgctattctatacaaggtatgtagcccaggaaatgaccaacctgatgtgtgttatgacccatctgagcctcccatgaccacagttttaaaaataagattaagtactgaggactggtgggggctcataaatgatatgagtaaagtgttagccaaaacagaaggaaagaaaagggattcccaaacaagtcaccttgaaatttgatgcctgtgctgtcattaatagtaataagttagaaacatgatgtggttctcttaattaggaaagaggctgtatggcagaaaataagtacatttgtcatgaattaggactgtgtggaaataaatgtagatactggtcttgtgtcatttaggctatgtggataaaaaataaaaagaatcctgtccaccttcagaaagggaaagtggcccttcctgtaccagtggtcagtgtaaccccttagaactagtaataaccaatccccttgatccttgctgaaaaaatggggagcatgtaaccctagaaattgatggggctggactggatcctcgagtaaatatcgtggtttgaggagaagtttataaatgctctcctgagccagtatttcaaatcttctatgatgaactaaatgtgccagtaccagaaattccaggaaaaacaagaaatttgtttttgcaataagccgagcatgtagcccagtctctcaatgtcacttcatgttatgtatgtggaggaactgtaatgggagatcaatggccatgggaagcccgagaattagtacctacagacccagttcctgataaattcccagctcaaaagactcaccctgataacttctaggtcctaaaagcctcaatcattagacaatactgtatagcaagagtggggaaggacttcacccttcctgtgggaagactcagctgccttgggcaaaaactgtataatagtactacaaaaacagccacctggtggagttcaaaccacactaagaaaaatccatttagttaattcccaaagttgcaaactgtgtggacccacccagagtcccaccaggactggacagcccccactggattatactggatatgtgtgcatagagcttacaccaaattacccggccagtgggcaggtagttgtgttattggcactattaaaccatctttcttcctactgcccataaagacaggtgaactcctgggcttccctgtctatgcttcctgcaaaaagagaagcatagctataggaaattggaaagatgatgaatggccccctgaaagaataatataatattatgggcctgctacttgggcacaagatggctcatagggattccagactcccatttacatactcaaccaaatcatatggttacaagctatcttagaaataatcactaataagactggcagagccttgactattctggcccagcaagaaactcagatgagaaatgctatctatcaaaatagattggctcttgacaacttgctagcagctgaaggaggggtctgtaggaaatttaaccttactaattgctgtctacacatagatgatcagggcaagcagttgaagacatagttagaaatataacaaaattggcacatgtgcccatgcaagtgtggcatggatttgatcctggggccatgtttgaaaaatggttcccagtgctaagaagatttaaaactcttataataggagttataatagtaatagaaacctgcttactgctcccttgtttgctacccatacttcttcaaatgataaaaagcttcatcactaccttagtttaccaaaatgcttcagcacaagtgtactatatgaatcactatcgatctgtcttacaagaagacatgggtagtgagaatgaaagtgagaactcccactaatgagtgagattctcaaagagggggaataagggaggagaccacccctcatattgtcttatgcccaatttctgcctccaaagaagaagaagtaaaaactaaaaggcagaaatgaaatccacaggcagatagcctggtgccgtgccctgggcctggttaaagatcaacccctgacctaatcagttacgttatctatagattccagacattgtatggaaaagcactgtgaaaatccctgtcctgttctgttccgttctgattactggtgcatgcagcccccagtcatgtaccgcctgcttgctcaatcaatcacgaccctttcatgtggacccccttagagctgtaagcccttaaaagggacaggaattgctcactcggggagctcagtttttggagacgtgagtctgctgatgctcccagtggagtaaagctcttccttctacaactcggtgtctgagtggttttgtctgcagctca

SEQ ID NO: 35 - DNA, 5829 bp, synthetic construct
  source 1..5829: mol_type = other DNA
  misc_feature 1..5829: note = HERV
tgtcaggcctctgagcccaagctaagccatcatatcccctgtgacctgcatgtatacatccagatggcctgaagcaagtggagaatcacaaaagaagtgaaaatggcctgttgctgccttaacagatgacattactttgtgaaattccttcttctggctcagaagctcccccactgagcaccttgtgacccccacccctgcctgccagagaataatcccctttgactgtaattttccactacctacctaaatcctataaaacagccccacccctatctccctttgctgactctctttttggactcagcctgcctgcacccaggtgattaaaaagctttattgctcacacaaagcctgtttggttgtctcttcacacggacgcccgtgacatttggtgctgtgacttcgatcgggggacttccctggggagatcaatcccctgtcctcctgctctttactccatgagaaagatccacctacgacctctggtcctccgatgaaccagcccaaggaacatctcaccaattttaaattgggtaagcggcctccttttactgccttctccaacctctctcactatccctcaacctctttctcctttcaatcttggcaccatccttcactctctcccttctcttaatttcagttcctttccttttctggtagagacagaggagatgtgttttatccatgaacccaaaactccggtgccggtcagggacttgggaagacagatttcccttggtgtttaatcactgcagggacacctgcttgattattcacccacgtttcagagatgcctgatcaccgcagggatgcctgccttgatccttcacccttagtgacaaacaccagtttcctagggtgcaaacacccccccaccccttctctccgtgtctctaccctctcttttctctgggcttgcctccttcactataggcaaccttccaccctccattcctccttcttctcccttagcctgtgttctcaagaacttaaaacctcttcaactcacacctgacctgaaacctaaatgccttattttcttctgcaatgctgcttaaccccaatacaaactcaacaatggttccaaatagccagaaaaaggcactttcaatttttccatcctacaagatctagataattcttgtcgtacaatgggcaaacggtctgaggtgcctgaggtccatgcattcttttacacatgggtccctctctagtctcttttcccaatgtgactcatcccaaatcctccttctttcccttccacccttcccctcagtcctaaccccaagcatcactgagtcttttcaatattccttttctacagacccatctgacctctcccctcatccccagactgctcctcctcaggtcgctccccaccaggctgaatcaggctccaattcttcctcaggctctgctcccccaccctataatccttctatcacctccccttctcacacccagtccgacttacagtttctttccccgactagcccccgccacctgcccagcaatttcctcttaaatcggtggctggagctaaaggcatagtcaaggttaatgctcctttttctttatccgacctctcccaaatcagttagtgtttaggctctttttcatcaaatgtaaaaacccaccccagttcatgacttatttggcagcaactctgagacgcttcacagccctagaccctgaaaggtcagaaggctgtcttattctcaatatgcattttattacgcaattcattcccaacattaaataaagctccaaaaattagattttggcccttaaaccccacacaggtcttaattaaccttgtcttcaaggtgtacaataatacagaagagttgcaattacttgccgctgctgtgagagaaactccagccacatctccagcacacaagaacttcaaaacgcctaagccacagcagtcaggcatttcttcaggacctcctcccccagtatcttgtttcaagtgctggaaatctggccactaggccaaggaatgcccacagcctgggattcctcctaagctgtgtcccatatgtgcaggaccccactggaaatcggactgtccaactcacctggcagccactcccagagcccctggaactctggcccaaggctctctgactgactccttcccagatcttcttggctcagtggctgaagactgacactgcccacttgcctcagaagccttctggaccatcacagatgctttcggtaactcttacagtggagggtaagtccttcctcttcttaatcaatatggagacaacccactgcacattacctgcttttaaagggcctgtttcctttgcctccataactgttgtgcgtattgacagccaggcttctaaacctctcaaaactccccaactctggtgccaacttggacaatattcttttatgcactcctttttagttatctccacctgcccagctcccttattaggccaagacattttaactagattatctgcttccctgactaggctacagccacacctcattgccacccttttccccagttcaaagcctccttcaaatcctccccttgtatctccctaccttaatccacaggtatgggacacctctactccctccctggcaactggtcacatacccattactatcccattaaaacctaataacccttatcccactcaatgccaataccccatcccacagcaaactttaaaaggattaaagtttgttatcacttgcctgttacagcatgcccttttaaagcctataaaccctctttacaattcccccattttacctgtccagaaactggacaagtcttacaggttacgtcaggatctgtgccttatcaaccaaattgttttgcgtatccaccctgtggtgtgaaacctcatatactctcctatcctcaatacctccctccacaacccattattctgttctaggtaaacctagctgaccccatagatcctaaatcctttctccactcccctatccattccttaaaaaacagctcccacactagctctccctaacttatcactccctttccactacacacagctgaagtgcagggctgtgcagttggagttcttacacaagagctgggaccacgccctttagcctttctgtccaaacaacttgacgttactgttttagcctagccctcatgtctgcatgtggcaggtaccactgctttaatacttttagaggtcctcaaaatcacaaactatgctcaactcactctctgcagttgtcataacttccaaaatctattttcttcctcacatctgatgcatatactttcttcccccctccactacctctcagcaagccgaactcattgccttaacttgagccctcactcttgcaaaaggagtacacatcaatatttatactgactctaaatatgccgtccatatcctgaaccaccatgctgttatatgggcaaaaagaggtttcttcactacacaagggtcctccatcattaatgcctctttaacaaatagtcttctcaaggccgctatacttccaaaggaagctggagtcattcactgcaagggccatcaaaaggcatcagatcccatcactcagggcaacacttatgctgataagatagctaaagaagcagctagcattccaatttctctccctaactgccagtttttctccttctcatcggtcactcccacctactcccccactgaaacttccacctatcaatctcttcccacacaaggaaaatggtccttagaccaaggaaaatatcccctttcagcctcacaggcccattctattcttttgtcatttcatagcctcttccatgtaggttgcaaaccgctagcccatctcttagaacctctcatttcctttccatcatggaaatctatcctcaaggaaatcacttcttagtgttccatctgctattctgctactcctcagggattgttcaggccccctcccttccctacacatcaagctcagggatttgcccctgcccaggactggcaaattaattttactcacatgccccaagtcaggaaactaaaataccttttggtctgggtagacactttcactggatgggtagaggcctttcccacagggtctgagaaggccaccgtgttcatttcttcccttctgtcagacataattccatgatttggccttcccacctctatacagtccaataacagacaggcctttgctagtcaaatcacccaagcagtttctcaggctcttggtattcagtggaaacttcataccccttaccatcctcaatcttcaggaaaagcaaaacagactaatggtcttttaaagacacacctcaccaagctcagcctccaacttaaggactgggcagtacttttacaacttacccttctcagaattcgggcctgttcttggaatgctacagggtacagcccatttgagctcctgtatggatgctcatttttattaggccccagtctcattccagacaccagcccaacttggattgtgccccaaaaacttgtcatccctactatcttctgtctagtcattcttctattcaccattcccaactactcataaatgccctgctcttgtttacactgccggtttacactgtttctccaagccatcacagctgatatctcctggtgctatccccaaaccaccactcttaactccctcttaaagtaaataatctttgctggcagggcaccctccaatactttcaccctgatgaagtcctattctttaccttaaactcactcttattctcgtttctgttcttatgccaccctctacctctccccagctatctctaccacactatcaatctcactcactctctcctagccatttctaatccttcttcaacaaacaattgctgactttgcatttctctttcctccaaaatcgccaaggccccgacttactgctaaaaaggggggactctgtatattttttaatgaagtgttgtttttatctaaatcaatctggcctagtatacgacaacataaaaactcaaggatagagcctaaaaacttgccaaccaagcaagtaattatgctgaacccccttgggcactctctaattggatgtcctgggtcttcccattcttagtcctttaatacctgttattcttctcttatttgggccttgtgtcttctgtttagtttctcaattcatccaaaaccatatccaggccatcaccaatcattctatacacaaatgctccttctaacaaccccacaatatcaccccttaacacaaaatcttccttcagcttaatctctcccactctaggttcccatgccacccctaatcctgctcacagcagccctgagaaacatcgcccattatctctccataccacctccccaaaatttttgctgctccaacacttcaacactattttatgttatttttcttattaatataagaagataggaatgtcaggcctctgagcccaagctaagccatcatgtcacctgtgacctgcatgtatacatccagatggcctgaagcaagtgaagaatcacaaaagaagtgaaaatggccggtttctgccttaactgatgacattaccttgtgaaattccttctcctggctcagaagctcccccactgagcaccttgcgacccccgcccctgcccaccagagaacaaccccctttgaatgtaattttccactacctacccaaatcctataaaacggccccacccctatctccctttgctgactctctttttggactcagcctgcctgcacccaggtgattaaaaagctttattgctcacacaaagcctgtttggtggtctcttcactcagacccatgtgaca

SEQ ID NO: 36 - DNA, 8968 bp, synthetic construct
  source 1..8968: mol_type = other DNA
  misc_feature 1..8968: note = HERV
atgggaaattttaattctaaaaaggccaattcccaatcacttagtttctagtcctcctttaaaaattacttcttcaaaatcccaagaaataaaagagatttgctaaagattcaactaagcaaaagtcctatcaagtatctttacatatactcaccacctatttgcccttctttcttggtgacctagctgtatgtcctttgcccatttttgttgggtggagagaagtttggtctttaatttttaagtactctctaaataataaagcaattagcttttgtgatgtaagttgcatttttttcttgcagtttatcagttgatttttcttaagctggttttttttccctctcttccatgattttaaattgaaattcaggttcatgtctatctcactccttatactgctgtcaaagtaaatgtggcaaaattcacctttaaagcaactattaaacagaattacatagaaatgagcattagtcatatataaagctattattaaatatccagaaatattctgatgttttctagaggtcattagcatggcccataatatcataacacatttttgttctctaaagatgtggactggttaaagaacacaaggtcacttattttaaaaatatcagaacacgtttattattttgatacattttttaaaagttgtgttttaaaaagttaatttagtatattctgaatccatacaaaagtaagctagtacttttataataacaattattctttcatttcaatttttattatgttttttatatagaacacagcacaaagtgcttttgaattaaagtttagaaaatatcgaaagcggccaggcgcggtggctcatgcctgtaatcgcagcactttgggaggccgaggcaggtggatcacgaggtcaggagatccagaccatcctggctaacatggtgaaaccccgtctctactaaaaatacaaaaaaattagccgggcgtgttggcaggtgcctgtagtcccatctactggtaatcccaactactcaggaggctgaggcaggaaaatggtgtcaacccgggaggcggagcttgcagtgagccgagattacgccactgcactccagcctgggagacagagccagactccatctcaaaaaaaaagaagatatcgaaatcttattttccaaatatttctcttcagtagtcttaaccactcttcccatgatcatttcatgcctttctagtaaattaatttttctccctagagaaagtaaaaactgatatagcatatatttcactctaaactgtttaaaagactagggttctgctctataaaataaaaggaaacaaactaagggggaaaagtacgtatgtaaaaaaacacatatgtgaaagaagataggcagaaaatgaatcaaaatattaggagtggttatcagtaggtagcataattataagtgacttatttttcataactttctgcattttccaagctcttcttactgagcccattccatattacaaatattttaaaaaagaacagtgaccaccttacaaaccagaaattccaggcaacaccttattcctttaagtaatccaaactattttctatctcctaaatgcagtcttttcccctgactagatactgtgtgcgtttagcacgaggcagaagggtgggggtttcacagtgacgcacacaaagaggcaggagctttgctaggtaccagggaaggggcagcatcttcaagtgcacatgtctgcgtctctgtgaagtcacttcaaagactttatgaagctcagatctgattctgttgaaatcaattggctcctttcccaccaaaattgtattcgagatgttcttcagctgtatagtactttgtcaaggaaggaccctgaaataacaggatgttttcatttaattaacagcatggtagacaactgaactacccagaatttacaggctgataagctttggatattttctaaactttattccacaataacaggagttgctttaggaggacaagttagaacatttggagggaaatgttataattgtgatcaaattggtcatctaaaaaagaattgcccagtctcaaataaaaaagagccacctgacttatgtccaagatgtaaacaaggaaaacactgggctagtcaatgtcattctaaatttgatagaaatgggcaaccattgtcgggaaaatgagcaaaggggccagcctcaggccccgcaacaaactggggcattcccaattcagccctttgttcctcagggttttcagggacaacaacccccactgtcccaagtgcctcagggaatgagccagttaccacagtacaacaattgtcccccgccacaagcggcagtgcagctgtggatttatgtactataaaggcagtctctctgcttccaggggagcccccacaaaaaatctccacaggggtatatggcccactgcctgaggggactgtaggactaatcttaggtagatcaagtctaagtctgaagggagttcaaattcgtactgatgtggttgattcagactataatggcgaaattcaattggttattagctcttcaattccttggagtgccagtccaggagacaggattgcgcaattattactcctgccttatattaaggttggaaatagtgagataaaaagaacaggagggtttggaagcactgatccaacaggaaaggctgcatattgggcaagtcgggtctcagagaacagacctgcgtgtaaggccattattcaaggaaaacagtttgaagggttagtagacactggagcagatgtctctatcattgctttaaatcagtggccaaaaattggcctaaactaaaggctgttacagggcttgtcagcgtaggcacagcctcagaagtgtatcaaagtactgtgattttacattgtttagggccagataatcgagacagtacttcccatgttaaaaaacatttattatcttgttttgctgtaatgggagttccagaaaaagatcaaaactgacaatggaccaggatactgtagtaaagctttccaaaaattcttacatcagtggaaagtttcacatacaacaggaattccctataattcccacagacaggccatagttgaaagaactaatagaacactcaaaactcagttaagcaaaaagaagggggagacagtaaggaatgtaccactcctcagatgcaacttaatctagcactctatactttaaattttttaaacatttatagaaatcagactactacttctgcagaacaacattttactggtaaaaagaacagcccacatgaaggaaaactgatttggtggaaagataataaaaataagacatgggaaatagggaaggtggtgacgtgggggagaggttttgcttgtgtttcaccaggagaaaatcagcttcctgtttggatacccactagacatttgaagatctacgatgaacccatcggagatgcaaagaaaagcgccaccgcggagatggaaacaccagtcacatggatggataatcctattgaagtatatgttaatgatagtgtatgggtacctggccccacagatgatcattgccctgccaaacctgaggaagaagggatgatgataaatatttccattgggtatcattatcctcctatctgcctagggagagcaccaggatgtttaatgcctgcagtccaaaattggttggtagaagtacctactgtcagtcccatcagtagattcacttatcacatggtaagcgggatgtcactcaggccacgggtaaattatttacaagacttttcttatcaaagatcattaaaatttagacctaaagggaaaccttgccccaaggaaattcccaaaggatcaaaaaatacagaagttttagtttgggaagaatgtgtggccaatagtgcggtgatattacaaaacagtgaattcggaactattatagattgggcacctcgaggtcaattctaccacaattgcacaggacaaactcagtcgtgtcccagtgcacaagtgagtctagctgttgatagcgacttaacagaaagtttagacaaacataagctaaaaaattacagtctttctacccttgggaatagggagaaaaaggaatctctaccccaagaccaaaaataataagtcctgtttctggtcctgaacatccagaattatggaggcttactgtggcctcatactgcattagaatttggtctagaaatcaaactttagaaacgagatcgtaagctattttatactatcaacctaaattccagtctaacagttcctttccaaacctgtgtaaagcccccttatatgctagttgtaggaaatatagttattaaaccagactctcaaactataagctgtgaaaattgcagattgtttacttgcatttattcaacttttaattggcaacagtgtattctgctggtgagggcaagagaagccgtgtggatttctatgtccatggaccaaccgtgggaggccttgccatccgtccatattttgactgaagtattaaaagccattttaaatagatccaaaagattcatttttactttaattacagtgattatgggattaattgcagtcacagctacggctgctatggcaggagttgcattgcactcttctgttcagtcagtaaactttgttaatgattggcaaaaaaattctacaagattgtggaattcacaatctggtattgatcaaaaattggcaaaccaaattaatgatcttagacaaactgtcatttggatgggagacaggcttatgaacttagaccatcgtttccagttacagtgtgactggaatatctcagatttttgtattacaccccaagtttataatgagtctgagcatcactgggacatggtcagatgccatctacagggaacagaagataatctcactttagacatttccaaattaaaggaacaaatttttgaaacatcaaaagcccatttaaatttggtgccaggaactgaggcaatcgcaggagttgctgatggcctcgcaaatcttaaccctgtcacttgggttaagaccatcggaagtactacgattataaatttcatattaatccttgtgtgcctgttttgtctgttgttagtctgcaggtgtacccaacagctccaaagagacagcaaccatcgagaaacagccatgatgatgatggcggttttgtcgaaaagggggaaatgtggggaaaagaaagatcagattgttactgtgtctgtgtagaaagaagtagacataggagactccattttgttctgtactaagaaaaattcttctgccttgagatgctgttgatctgtaaccctacccccaaccctgtgcttcctgaaacatgtgctgtgtcaactcagggttaaatggattaagggctgtgcagatgtgctttgttaaacaaatgcttgaaggcagcatgcttgttaagagtcatcaccactccctaatctcaagtacccagggacacaaaacactgcagaaggcagcagggacctctgtctaggaaatccaggtattgtccaaggtttttccccatgtgatagcctgagatatggcctcatgggaagggaaagacctgaccttcccccagcctgacacccgtaaagggtctgcgctgaggaagattagtaaaagaggaaggaacgcctcgttgctgttgagacaagaggaaggcatctgtctcctgctcatccctgggcaatggaatgtctcggtgtaaaacccgattgtatattccatctactgagttaggggaaaaccgccttagggctggaggtgggacatgcaggcagcaatactgctctttaaggcattgagatatttatgtgtatacatatctaaagcacagcagttaattctttaccttgtttatgatgccgagacctttgttcacgtgtttacctgctgaacttctctccactattatcctatgaccctgccacatccccctctccgagaaacacccattaatgatcaataaatactaagggaactcagaggccggtggatcctccgtatgctgaacaccggtcccctgggcccccttttttctttctctatactttgtctctgtgtctctttcttttccaagtctctagttccacctaatgagaaacatccacaggtgtggagggccaacccaccccttcagcattccatataaattacacggtcattagaagtgtttattgccctctgtgatggctgctatgtaggacatagaaagtatggcgaaaatggagacattagtagaaaacagagagattaatgattaacttgaccttttccttttttaattttgtccaaagaaaaagtgttctcagagttgagctactaaacagcctccttccattctagaaaacgtacaggtcacccataacagaaaaaatgtttttttgattctgttgttctacaccatggtggtagcaggggacagaaataggaactggtcatcttggaggggtcagcttcagaaagcatgtcctcctcttctacttacagttaagaatgagctgcagtagtggatttagtacaaggtatccacgtgcttccccagccgtgctgaagcgtctccttttaaggccgggaatggtgtccttccaccaagggacacccctcctgatgaggcctccagctcatctgttccccggttattactgccaccagattcctcatcttcgttctccttccatttcacaggacttcccagcatcacctgttacctgtattcctccaaatactctcatgtccaacacacagctatgtctttggagcctgcctctttactccgccccagaacctgggtgcacaaggtcttaacgcttggcacagattctaactcacttcttgactagcatattttgccacagatgattcactttgttcccatggaacactgtggtcaaaattctaatcttaaaagggaaagttgaaacaaaatttttaaggactttatacctaccgaaaagtgatcagtagtcagtgtacacaatttgaggtgtactttgatgtaagagtatttccaaaaagttgaggataaatgaaattttcataaattaattttgtcaggcctctgaacccaagccaagccatcgcatcccctgtgacttgcacgtatacacccagatggcctgaagtaactgaagaatcacaaaagaagtgaaaatgccctgccccgcctttactgatgacattccaccacaaaagaaatgtaaatggccggtccttgccttaagtgatgacattaccttgtgaaagtccttttcctggctcatcctggctcaaaaagctcccccactgagcaccttgcgacccccacttctgcccgccagagaacaaagccccgttgactgtaattttcctttacctgcccaaatcttataaaacggccccacccctatctcccttcgctgactcttttcagactcagcccgcctgcacccaggtgaaataaacagccatgttgctcacacaaagcctgtttggtgatctcttcacagggacgtgcatgaaaaatttctcaattgattaacgtgctcagttgattactgaacatttctcatttgattatactcagagattggggagatctttaaaaaattgtctctattcatcagaagttaatttagcatggaaaagaaaacacacataagcacaatgtatggcacattgtaagtggctaagaatgtttgaagaataaataagcaactacatgaatgcaatagagttaacaggcaaaaaacaacacaggaaatgtcaattactataggttatgtactgttataatgatgggagaaatgtttgtcatttggtaaaaaaagcaggattcaatattgtgcaatcaatataatctcaaggtgtatatgcaaatgtctaacaatatgcaaagaagcttaagaatactactagatagagattattgcaggtaatttttatattttttatattttcccctatttcttgtttttaacaatgatgagatattactgttaaaataaattagtttattttttaaatggtgtagtcaggaaatgtgagtttcaaagagagatatcattttggactagagttgtttgggagagcatcttggttgattgaattcagagctggagctgggttcagggaatttagatagaaagaaaggcggaaaagtgttttaggcaggtaatctgacaggaatgaggctaaatcaaataatttatcattgtggaatatttttaaaatgaactcttgtaaaaatggttgttgttcttccagtctgctgttatttcgtctgtttgtaaatgagtggccacacagctggccctgaatcagttctgcctgacttttcccccatagcactctgatagtaaaggaaaatgaatgcctcacgcaagaactttggagacatagtctaaagcatccctgaaagcacaatattttctgtcttattttatcnnnnnnnnnnnnnnnnnnnnnnnnnnnngagacggagtcttgctctgtcacccaggctggagtacagtggcgcgatctcggctcactgcaagctccgcctcccgggctcacgccattctcctgcctcagcctcccgagtagctgggactacaggcgcccaccaccacgcctggctaaattttnnnnnnnnnnnnnnnnatttagtagagaaggggtttcaccgtgttagccagaatggcctcgatctcctgactcatgatccactcgcctcggccgcccaaagtgctgggattacaggcgtgagccacagcgcccagccgtcttattttatcttaaacttcctgcattatttatttgtcttttcctcccttataaatgtgtttgtgttcatgttgaaataatacaatttttatttgaactattggagtaatattagaaaaatatattctctaggaaaccatgccattttcatcttatgatttttatatccccttggtatattagcattagccatgtatcccccagtgtatacatatgccca

SEQ ID NO: 37 - DNA, 6815 bp, synthetic construct
  source 1..6815: mol_type = other DNA
  misc_feature 1..6815: note = HERV
tgtgttttcttttcttttttaagagacaggtgtcaggcctctgagcccaagctaagccatcatattccctgtgacctgcacatacacatccagatggccggtttctgccttaactgatgacattccaccacaaaagaagtgaaaatgtcctgttcctgctttaactgatgacattgtcttgtgaaattccttctcctggctcatcctggctcaaaagctcccccactgagtaccttgtgacccccactcctgcccaccagagaacaacccccctttttcctttaccttcccaaatcctataaaatggccccaccccatctcccttcgctgactctctttttggactcagccaccagcacccaggtaaaataaacagccatgttgctcacacaaagcctgtttggtggtctcttcacacggtcgcgcatgaaatttggtgccgtgactcggatcggggaacctcccttgggagatcaatcccctgtcctcctgctctttgctccgtgagaaagatccacctacgacctcaggtcttcagaccaaccagcccaagaaacatctcaccaatttcaaatccagtaagtggcctctttttacttacttctccaacctccctcactatccctcaacctctttctcctttcaatcttggcgccacacttcaatctctcccttcttttaatttcaattcctttcattttctggtagagacaaaggagacacgtttcatccgtggacccaaaactccggtgccagtcacagactgggaaggcagccttcccttggtgtttaatcattgcagggacgcctctctgattattcacccacatttcagaggtgtcacactacacagggacacctgccttggtccttcacctttagcaggaagtcccacttttctgggggaggggcaagtaccccaaccccttctctccatgtctctaccccttctccgcctttctggggggcaagaaacccccaaccccttctccttcacacttagcagcaagtcccacttttctgggggaggggcaagtaccccaacctcatatctctgctccccgatcccttatttctgtgccctgacctcttacatctctgcaccccaatcccttatttccgtgccctgacctcttatctctgcgccctgatcccttatttccacgccccgacctcttatctctgtgccccaaccccttatttctgtgccctgacccctttcccgcttttctggagggtaagaacccctgaaccccttccctctgtgtctctactctcccttttctctgggcttgcctccttcactatgggcaactttccaccctccattcctccaactcccttagcctgtgctctcaagaacttaaaacctcttcaactcacacctgacctaaaacctaaatgccttattttcttctgcaatgccgcttgaccccaatacaaactcgacagtagttccaaatagccagaaaatggcactttcaagttttccatcctgcaagatctaaataattcttgttgtaaaataggcaaacggtctgaggtgcctgatgtccaggcattcttttacacatcagtcccttcctagtctctgtgcccaatgcaacttgtcccaaatcttccttctttccctccctcctgtcccctcagtcccaaccccaagcgtcactgggtctttctaatcttccttttctccagacccatctgacctctcccctcctccccaggctgcttctcgccaggtcgagctaggtcccaattcttcctcagcctccgctcctccactctataatctttttatcaccacccctcctcacacccagtctggcttacagtttcattctgcgactagccctcccccacctgcccagcaatttcctcttaaaaaggtggctggagctaaaggtatagtcaaggttaatgctcctttttctttatccgacctctcccaaatcagttagcatttagactctttttcgtcaaatataaaaatcccagcccagttcatggcttgttcggtagcaaccctgagacactttacagccctagaccctaaaaggtcaaaaggctgtcttattctcaatatacattttattacccaatctgctcctgacattaaataaaactccaaaaattaaattctggccctcaaaccctacaacaggatttaattaacctcaccttcaaggtgtacaataatagaaaaaagttgcaattccttgcctccactgtgagacaaaccccagccacatctccagcacacaagaacttccaaatgcctgaaccacagcggccaggcatttctccagaacctcctctcccaggagcttgctacaagtgccagaaatctggccactgggccaaggaatgctcacagcccagaattcctcctaagccgcgtcccatctgtgcaggaccccactggaaattggactgttcaactcacttggcaaccactcccagagcccatggaactctggcccaaggctctctgactgagtccttcccagatcttctcggcttagcagctgaagactgacactgcctgatcaccttggaagcctacaggaccatcacagatgctctaggtaactctcacagtggagggtaagtctgtccccttcttaatcaatatggaggctacccactgcacattaccttcttttcaagggcctgtttcccttgcctccataactgttgtgggtattggcggccaggcttctaaacctcttaaaaccccccaactctggtgccaacttagacaatactcttttaagcacccctttttaattatccccacctgcccagttcccttattaggctgaggcactttaactaaattatctgcttccctgactattcctggactacagccgcatctcattgctgcccttctccccaacccaaagcctccttcatgtcctcctcttgtatccccctaccttaacccacaagtataagatacctctaatccctccttggtgaccgattatgcaccccttaccatctcattaaaacctaatcacctttgccctgctcaatgccaagatcccatcccacagcacgctttgggcattaaagcctgttatcactcgcctgctacagcatggtgttttaaagcctataaactctccttacaattcccccattttacctatcctagaacaagacaagccttacaagttagttcaggatctgcgccttatcaaccaaattgttttgcctatccaccccatggtgccgaacccatatactctcctatcctcaatacctccctctacaacccattattctgttctagatctcaaacatgctttctttactattcctttgcacccttcatcccagcctctcttcgctttcacttggactgaccctgacacccatcaggctcagcaaattacctgggctgtactgctgcaaggcttcacagacatccctcattacttcagtcaagctcaaatttcttcctcatctgttacctgtctcagcgtaattcttataaaaacacacgtgctctccctgccgatcatgtctgactgatctctcaaaccccaacaccttctacatcctaggcatggttagatactttcgactttagatacctggttttgccatcctaacaaaaccattatataaactcacaaaaagaaacctagctgaccccatagatcctaaatcctttccccactcgtttttctgttccttgaagacagctttagagactgcccacaaccgagctctccctgactcatcccaacccttttcattacacacagctgaagtgcagggctgtgcagtcagaatttttacaggactgggatcgcgtcctgtagcctttttgtccaaacaacttgaccttactgttttaggctgaccatcatgtctccgtgcagtggctcctgcccccctaatactttcagaggcctttaaaataacaaactatgctcaactcactctctacagttctcataacttccaaaatctatcttcttcctcatacctgattcatatactttctgctccccagctccttcagctgtactcactctttgttaagtcccacaattatcattgttcctggcccagacttcaatctggcctcccacattattcctgataccacacctgaccctcatgactgtatctctctgatccacctgacgttcaccccatttccccacatttccttcttccctgtttctcaccctgatcacacttagtttattgatggcagctccaccaggcctaatcgccacacaccagcaaaggcaggctatgctatagtacaagccactagcccgcctcttagaacctctcatttcctttccatcgtggaaatctatcctcaaggaaataacttctcagtgttccatctgctattctactactcctcagggattattcaggccccctcccttccctacacatcaagcttgaggatttgcccccacccaggactggcaaattagctttactcaacatgccccgagtcagataactaaaatacctcttagtctaggtagacactttcactggataggtagaggcctttcctacagggtctgagaaggccaccgcagtcatttcttcccttctgtcagacataattcctcagtttagccttcccacctctctacagtctgataacagaccagcctttattagtcaaatcagccaagcagtttttcaggctcttagtattcagtgaaacctttatatcccttacggtcctccatcttcaggaaaagtagaacggagtaaaggtcttttaaaaacacacctcaccaagctcagccaccaacttaaaaaggactggacaatacttttactactttcccttctcagaagtcagacctgtcctcagaatgctacaaggtacagcccatttgagctcctgtatagacgctccgttttattaggcccagtctcattccagacaccagaccaacttagactgtgccccaaaaaacttgtcatccctactatcttctgtctagtcatactcttattcaccgttctcaactactcatacatgccctgctcttgtttacactgccggtttacaccgtttctccaagccatcacagctgatatttcctggtgctatccccaaactgccactcttaactcttgaagtaaataatctttgctggtaggactatgctgaatctccttaggcactctctaatcagatgtcctgagtcgtcccaattcttagaccttttatacctatttttctccttctctcattccatttagtttttcaattcatacaaaactgtatccaggccatcaccaataattctaaatgacaaatattccttctaacaaccccacaatatcaccccttaccacaaaatctcccttcagattaatctctcccactctaggttcccatgccacccctaatcccgctcgaagcagccctgagaaacatcgcccattatctctccataccatcccccaaaattttcgctgtccgaacactttaccactatttcattttatttttcttattaatataagaagataggaatatcaggcctctgagcccaagctaagccatcatattccctgtggcctgcacgtacacatccagatggccggttcctgccttaactgatgacattccaccacaaaagaagtgaaaatggcctgttcctgctttaactgatgacattgtcttgcgaaattcctcctcctggctcatcctggctcaaaagctcccccactgagtaccttgtgaccccctcagtcctgcctgccagagaacaacacccctttttcctttacctacccaaatcctataaaacggccccaccccatctctcttcgctgactctctttttggactcagcccgcctgcacccaggtgaaataaacagccatgttgctcacacaaagcctgtttggtggtctcttcacacggacgcgcatgaaaacagggtctctgtctgttatccaggcaggagtgaagtggcacaatgatgactcactgcagcctcaaactcctgggctcaatggaagcctccccccttagcctcctgagcagctaggactacaagcatgcatcaccacacccagctaatttttaaaatttatttttgtagagatgaggtcttgctatgttgcccaggctggtctcaaactcctggcctcaagtgattctcctgccttggcctcccaaagtgttggaattagagatgtgagccaccacatctagcctaaatttgtgttttcagacataaaattgtcacctttcaaaaaggagctctctgttgtgctaaaacagcccactgatgacaaagaaaccactcactagcaaggcaccaaaccaagtggtgagtgaaaagaagtgccaggttactaagcccataaatttgagatacaaaaaccagtggggaacactagagaggcagatctagagactgaggtggaaacctctggaaacacaaaaggtatctgagaactagactgtgactcttgagaattacaaaaaggtcagacacgccaaaaataaaccctccaaaagctgttttctaagcatctcaccaggcttcagttgcaccccatgtggctttatgggacccagggcagagttcatatcacactcagctgaaccctggttgacctttggagtcatagctcataaatcatccctgttttcccaaatttcggggcacaaattgtacctttctccaagggcacccacttggagacaatctcctactttgaggaactgtgatattttcctagtcttgtaaaatagtattactttctggtcctccagggaggagaacaagggacaagtg

SEQ ID NO: 38 - DNA, 5854 bp, synthetic construct
  source 1..5854: mol_type = other DNA
  misc_feature 1..5854: note = HERV
tgcagattcactgagccagacaaaggcatgaatgactgttttcccctaccctcttctcacatgaaaattatgtatttctcaatatccttccctttcccctttagatattgaaaccctcaaaatcatctatggggaaaggaatagacctatctcccaggtgtgtatccttaactttggcaaataaacctcaaaaaatgattgagattttcctgggtcattttccttgattgaccacggagggatcctgagtgaaggtggccctggcctgcagcagctctcctattggtaccagattgggctctttatagcccaaaccaataagacaatttgttgaggtctgggacctcctccctgcaggaagttggggctctgaggtttaatttgatgtttnnnnnnnnnnnnnnnncacctcnnnnnnnnnnnctgggagtttttgcttgcttccatcagggaaggcaagcttgtctgcttcttcatcagtggagaacagtcttcagcttgagtctcatcactaggtaaggaggtgagttgggaatctgtcttgcaaattctctttaataactaaaggtactgttaacaaccagctggtcttaatttctccttacatttagagcactcaaattgtataatttgtgtgattgctgttcgttttgcttagatgtcttatttacttgtttctgtcttgttaggtgtttttttgtatgcttcagtccttctcttattggatttggccaactctaaaccctctagcttatagtatggaattttcctctccaaagaaataagagctcgcccttctcagcctttcttaggcaactgagaattacatgagggtgtctggaagaaacactccctaagatgtgcaacggctctgagtaggatttcccctcagaagaacgtacttagggtgtaatctcagctagcaggtgcaaataaggagctgatctcccatgccttgagcccctgacacactgtgccaggtagccacgataccagttcagcgagtaacggccctgaaaagctaggtccccaagcagcacactttggatccaacaaaccctccgacttggtcaaatctgaaggaaaactctaaattatgtggaaggagccctttaagttggctgaaacccccacagctgtggaacacaaagttccacccttagaaactctggccaggtatatgcaaaaacacttatggtgaattgtcatgcaaatatttaatcaagtggaccactataatcaaggcagattctaacctacagaggcctagatggggatcttctgagatgcccaaattagtgtacctgtgaactaggatgcaaaacgcaggcacaaaactaaacaaccagaatgggagagctactttcaatggtacctagagagtagcaaatggggggaagatcacctcatgtccttacaataagccaacaaacaacttagaaatgctaaccaaggactctctaaagttctatctctcttaaagaaatccccagaatcccttacttccctgtggcacctccccacctccacttacacccccactttacccctgacctctctgaacttcccggaccagatctgttccctccttctcctgcatgttctaatttaacatcttcctctccttgtacaccactcactcaacagaaggcagtagggagtctaacttccaagaccctacctcctgacaattccctgacagtgccagtggcagcctctcatctggaagatgtggaaaagggggaccctgcagggccatctctcatgatcaccctgttttgggaacaaccggtaacacataggggaaccccggtgattgtctaccaacgctggtcaaaggctgagttgcagggcataaagaatttctgggcccccataagatccaattgagtttgcccaagaatttgaattcattatcagaacctatgacccaggccattcagacctctaacagctggcacacatgttggtctcggaagctaaagctaaggaatagctggacaaagtacagtggtcagaccctgtagcagacttaacccttgaaggcccaatagagctacaataaccagcccccagaagtcgagaacacagacacaaagatgtgtgggaatgagcaactgctctgttaaataccatttcttcaatattccaaagggttgtggattgggataaaatccagcaatgccaccagaaccgaaatgaatcaattttagattattttatacattttgactgagataatatgctccaaataattaacttacactctctcgcctggtccttctgacgaaacccctcatgactatgttctgatgactgaccaacttctcacttctgggacagacttacaagagatgccactggataatgctgagatagaatggtatacagatgggtcttatttaagaggatggggatggaaattttagagcaggatatgctgtggtctctttactagaggtagttgaagctggtcctcttcctcaagccaaatcagctgaaggggccaaattgattgccctgactcaagcttgtcgattagcaaaagacaaggctgcaaacataccctgacacctgctaagcatttggagttgctcaggacttcggaatgtgaaaggagagaggatatttaacctcctcagggcaacccataaaaaatggacaaccaactctcagagctgttagaaactattctcaaccaaaacttttgacagttgtaaaaatcccaggtcactctacattggacaccactaaaagtggagataacaaatttgctattgctacagctgaaaaagcagcattcaagccaccagacccaatccaaaaaaatgatcataaagcctgaaacacttaaaaacatgttgaaggaaacctagagtatagccccaacaaaagagaaatccacttggaaacagacaggggaatacctgtctcctgaaactaaaatattgtatggacttaataataaaccattattccaatgggatgtcaggtgccccttaaggaatgtgttaataatctgacacgatggaatccagataaaagtatatcctagtgtaaacaatgttactggaaacgatcctccacagtggcacaaaaggtttattcccagtgtgctatttgtcctaaggataatccacggaagctcctccatgaggcccagggtcattttccccttccagctggaccttttgaggtatggcagcttgattttatccagccgccatcctctcaaggttacatgtcttagtaatggtctgcatgttttcccattgggttgaagcttttccctgcaggcaagcaacagcgatggcagttggaaaactttattagaaaaaattattccactgtggggagtccactgtgaactttacgtgatagaagaactcattttcctggtcaagttattcaaaatatttgtgaaatttggcccatatttcaacatttccattgtgcctaccatccccagtcctcagagctggcggaaaggaccaatggaataattaaaacacaattggctaagttcataaatggacttcacctctgttggcctaaagcactcccctggtgcttcttaccctctggtccaccttgggaaacatcaactatccccttatgaaattataacaggaaggcccatgtgtatgggaaccaaaataaccaacccaacttttctcaagagagatatattgcagtattataagggactcatttgtcatctttacaaaagccaagatttggtaaagaattcctttcatagtccactccctgaagacaaggttcctggttatgatctgcagcctgaagactttgtctattggaaaagacatttaataaaggactcccttcaaccccaatggaaggagccataccaaatactattaacaaatccatgtgctgcaaaaactagacagtataaactcatggatttacacctctcatcttaaaaaggcacaacctcctgagtaggctgtaactcctaccaaggatcttcacctgcagctcattaacattgaacttcaatccaggactaggagcagaccatagctgttgtggactgcttaaacctaagacacaggaccaggcctgtatacaaaagaacacctatgaaacccaagacacaggaccaggcctgtatacaaaagaacacctatgtgtattgtatgatagctattacaattattgtcctagagatactggcaactgctatcttgtgcagaacagggcatttgccttgtctgatttaatgtccttttagtagttaaaatgaatttcacaaccttgctcctatttaccctatgtctctaaaacctcttgccactgtcacccattgttgcccataagacaaacctgtttctacaatgggctcaggattatgcagacagattataaaaggatgcctgtgtgatatgcagactcatgcctctttccagtggctccagcctgccatggtgggtatttcccttacaaggtcaagactagctagaataccaaaatttattacatcacagaaatggtttcgtatccttagtgctggcataacaaaagactaaatatatataatttccccattaaaaacactcaagagcaaggacatgggaaaagattttcaagggaaaggaccagctcaccagctctcactttagcattcccccaactaaaagagaaggtggcaaccatgccccaaacaacagcccattttcaaaatgggataatgcaaatttggaatgggtttatctggttcatcccttcatttggccaactcagccaaaatgctcctttatgcttggaacgaagaaacgaccccaaggaccaatggccaaacactacaagagatatggggtggatacctggagaaattgtattgtgacctggagaaattggtggcagaagctattgcttccnnnnnnnnnnnnnnnnnnnnnttgtgtcatgtcctgtttttgcttacaatgttattatggtatgtccagattagtcatcaactgaaagggctagaataatgattgcccaaagagttgctctaattgaggaggcagtagcatagcctgacccagcttccaggtttgctttcccttttgctgtatatctggtctagatccctatatttttaatttcctcttaccttctttcctccccctattttaatcttcatggggcacgacctgctaagaatgagccttcctagcaaggtgggacctgaatttctgggaataaaccatcctagcaatgaaggaccagctcnnnnnnnnnnngaaaaagaaaaaagcaagctgagaccagagacacattttccttctaaaatgctttctccaaatgattttaaagaaaaaatgggggaaatgtgaaaataaatcttgggaccccaaactcactaagccaaaaggaaaagtcaagctgggaactgggttatgcaagcctgcctctcattttggttcctaaataagatggctacaagatgaaaagctacatacctccctcacaaggaaattccttgtggtccccaagatcttcaccctaaagcatttctgttaaagtgcatcatgcaaatgtgaattgatagcttttcttcacagctgcggggaacataggacagaactcaaagtcatccctctgcacacattgtttcctctgccctattgtctacattaatcttatgtaaaaatgcagattcactgagccagaccaaggcaggaatggctattttctcctacccctctcacatgaacattgggtatttctcaatatcctgccctttcctctttaaatattgaagccctcaaaatcatcttcagagaaaggcatagacctgtttcccggttgtgtgtccttaactctggcaaataaacctcca

SEQ ID NO: 39 - DNA, 17038 bp, synthetic construct
  source 1..17038: mol_type = other DNA
  misc_feature 1..17038: note = HERV
tggttcagtgggtgaaggccaactatttgaagggtttcctagaacatgagacaggagagaggtgaggaaatgagggtgtctgtcctccactcagtggaaatctttgaggatggttcatggccaacactctcttatctaatattgagccctgggagtcctgggatccttttttccataatttttttatatgacacccactgtcttgagacttcaagatataaagagaaaacaggagcatcacactacctgatctcaaaatatgttacagagctgtagtaagcaaaatagcatgacattggcataaagaaaggcacatagaacaacggagcagaatgaataacacagatatattccatgcatttacatccaatggttttttattttttcttttgagatggagtcttgctctgtcactcaggctggagtgcagaggtgcaatctcggttcactgcaacctcagcctcctgggttcaatcattctcttgcctcaaattcctgagtagtggtattacaggtgctgaccaccatgctcagctaatttttatatttttagtggagacgatgtttcatcacgttggccagactaatcttgaactcctggcctcaggtgatccacccacctcgggctcccaaagtgctgaaattgcaggtgttagccaccaagcccagcccatccaatggactttgacaaagatgccaagaactcacaatcaggaaaggacagtcttttcaataaacagtgcagggaaacctggacatctacatgcagaggaatgaaactgcaactctacctgtcaccatacacaaaaatcaaatgaaaatggattaaagatgtgagtctaaggcctgaacctatgaaacacgtagaacaaaatattggggaaatgctccaggacgtttgtctgaaggaagacattttgttttaaaccttcaaaacacaagtaatcgaagcaaaaatagaccattgggattacctcaaactaagcaacttcagcactgctaaaaataaaccaacaaagtgaagagacaacccacagattgggagcaaatatgtgcaaactatgcatctgagatgggattaataactagaaatataagaagctcaaacaactcaataaaacaaatgatttaattgaaaaaggagcaaaagacatgaaatttccccacatacgaaaaagtgctcagtatcactcatcatcagagaaacgcaaattaaaatcaaagtgagttttcatctcaccccattaaaatggcttttaggccgggtgaggtggctcacttgtgtcatcctagaactttgagaacctgaggtgggtgaatctcataaggttgggagtttgagaccagtctgacccacatagagaaacgctgtctctactaaaaatacaaaaattagtagggcgtggtggcgtgtgcctgtaattccagctactcgggaggctgaggcaggagaatcgcttgaacctgggaggtggaggttgtggtgagccgagatagcgccactgcactccagcctgggtgagaagagcaaaactccatctcaaaataaaatgaaataaaataaaatggcttttagctgcaagacaggcaaaagaaatgctggcaaggtggtagagaaaggagaaccctggtaccctgttgggaggagtgtaaattagtacagccattacggagaaaagtatggaagtcctttaaagaactaaaaagaggttgggtgaggtggatcatgcctgtaatcccggcactttgggagactgaggcgggcacctcagttgaggtcatgagtttgagagcagcccagccaacatggggaaaccgcatctatactaaaaaaaccaaaaagtagccaggcatggtggtgtgcacctgtaatcccagctactagggaggctgaggcaggaaaatcatttgaacccaggaggcggaggttgcaatgagccaaggttgcaccactttgactccagcttgggctaaggagggaaactctttctcaaaaaagnnnnnnnnnnnnnnnngagaactttcatagtatccagcaatttcactactgggtttatatccaaaggaaagtaaatcaacatatcgaagtgatatctgcactcgtatgattggtgcagcactgttcacagtagccaagatgaggagtcaacctacctgcccatcagtgggtgaatggatagagagaatgtagtacatacgcacagtggagactactcatccatagaaagaataacatcctgtcatttgcagccacatggatggaactggaggtcattaaaaagattcccatttctcacccatatacaggagctaaaaggtggatctcatgaaggtagagagtagaatggtggctactggaggacaggaagaaaagggtggagggtaaaaaaaatgnnnnnnnnnnnnnnnnnnaaaatgtatttatgaccactagactttacacttaaaaatggtaaatgtggctgggcctggtggcccatgcctgtaatcccagcactttgggaggctgatgcgggtggatcacgtggtcaggagttcgagaccagctcgaccaacatggtgaaaccacctctctactaaaaatacaaaaagtagcctggcgtggtggtgcgtgcctgtagcactagctactcaggtggctgaggcaggagaatcgcttgaacccaggaggcggaggttgcagtgagctgagattgtgccactgcactccatcataggggacagagctagactccacctcaaaaaaaaatgttaaaagtggtaagctatataggtatatttatcctcaataaatatttcttcaaagaaaagtaaagggtgtaggggttgctggtgatgacatctctgtgtgggtgagaggccaggatgggcttctgggaaatgggtaaggttgaggggctgagggaacctctgatctccccaaactgagcccagtctccctcctctgggtctctcctgaccgctttctccatctgcctgggtgcctggagccctggccgtgggcctccatgcaggccatgtaggagggtttggaggtgccctgtcggccatcctgtgccctgatccctccctcacaccgaggctgcgtcttctctctgcatctgtccatgcttctctccatcctcagcaggaagctcctcagctaaggctctaggatcataggacatgggacagccatgggctttcctcacctgtgacagaaacaagcagtgggtcacttgactttgaccactcgtatggagagtcatggaaagagccgaagcatctgtaggtccctccgtgggtggcagggcccagaggaaagtcagcctggaatgttccgttgaccttgggccctgcagggagcctacgttcatgggcctccccttccctggatagatggtacatgtcataggagctccgggagctgcaggacaaggtcacattctctcctgccagaaccgtggggcccagctgggctgagagagaaggtttctcatatagacctggaaggagaagaggcagtttcctcagggaggatcttctttgtcacagctcccttcacctgagctgagaactcactcccctgttctatgacctaatgcnnnnnnnnnnnnnnnnnnaccctctaccccatcgctcttcatgtctatttcctccttccaccttctctgtctctctaggtctctgacctcacttccccacctctagatatgttttctctttttggattgttttattctctctgactctccttggattggttgacttgatgttactttttttaattctgagtttctcactttgtgtcctgttcataactttctgcatatttctatctattatctatcgatctatctatttatctattcggtgcctatctacaaattctctacctgtcatctatatctatatatcatctatttatccatcaattgtctatctatccatcaatcatctattatctatatctatgtatcatctctctctctctatgatttctctatgtctgcctctgtatctctatgtattatctatctatctgtcttcatcatcatcatctctatgtctcatctattaatgaatcaatcaatcatcatctatgtatctataacctattatctatcatctacctatttatcatctatctatatctatccatctatcatctgtcttgctctgcctctcggtctctctagttctctttggaatctctgcaattcatccccacatctccatctttcaatgtccttgtgcctctccctcaggagtctaattttagtgcttttctctgctcccttccatcattctcaccactcctctgccctcttttctctctctttatgtgtctgtgagtctctcaatctccttcctctggctcattctctgtgtgtttatgtctttgctttttggtgtccctgatttctctctgtgcctctcactgatcctctcataagtgggcttatttggaatatgagcctcagaatccagtctggagactacaagttcacacagcatacaggggttggtgttgtggggccatgatatcctgggacgattactctccattacatggaaggcagaggtgtcagaataaacatggcatctgtaggtgccacaaggcctgaggccacagggcccaactcaggtcagaaatatgggtgtccttgggttctcctggtagagaacactttgtggaggtaaaacagaaatgaaacttctaacctgtgccaggtctctgagcaaagtcagcatggagggacacctctctctgggacatgtctgtctgtgtgtctcctttaactctttctgtcttttctaactcccggtatggcccctgtgtctgttctctgttatgacacctggtctctacttgtgtctcctgtttctctgtctctgttggcacagacctcaccaagtcagtctctctccataagaataccaagctcatcttccttacagccacctgggtctccaattcctggatcattcactctgcatcccaatgacaatgagaagaaagtctggacactctcacctatgatcacgatgtccagagggtcactgggagctgacacctgatagggggagtgagtaacagaaccgtagcatctgtaggtccctgccaggtcttgcgtcatgcgactgatggagaagttggccttggagaccccatcatggtgttctccaatgaggcgcaaagtgtcgttaaacatcccctctctgtgcagaaggaagtgttcaaacatgacatctgaccaacactgcaggatgactgtctcttctgatttcaccaggcgacctgggtgggccaggagggaaggttttctgtggactcctaggaagagaggttgtgagtttagaaggtgtctctctttatcatcccatccatggcacctggattgagtcaggcttccccttcctggtgtcttatctctctccttcctctctgtgtcttcatgttcttttctgtgcccataactcctggtgcaggtccttccatctgtctccctcactcttctctgtccctctgtctctagtagcctctgattcccttgccgctgggctcagcctcatctcttgggctgttgtatctatttcgaactaatgtctttcctgctgtctatgtgggggtggaagaggaaccaggataggctgcacatccaggctcttagcagcctggttcaatctcttttggacgaattggaatccttggcaggaggtatgaactgatcagtaaggcaggcaccagtggccacacaccctgttcctggtagggactgggagccactcttgccatgccagtgccagcttccataggctggctcctggtgctggttggaggagtatcaacccctccctatgtggatggagcctggtggtggcatcatcatctgagccttgctgatctcagtgtagccaaccttctccttgtttggtttctttnnnnnnnnnnnnnnnnttggcgacagagtctcactcctttgcccaggctggagtgaagtggtgtggtctaggctcactgcaacctctgtctcctgggttcaagtgattctcctgccctcagcctcccaagtcgctaggattacatgcacctgccaccatgcctggctatccttgtgttgtttcttaacttgtccttgacctgggttccagtgttggtttcctgttgctgctgtagaaaattatcagaagcatggcaccaggagagagcacactaaccccttccaattctggagacagaaatcggaccctgtttgtcgtgggtaaaatcaaggcacctgcagggcttcgttccctctggagactcaggagaatcagttccttgacttttccagcctctataggccacctgcattcatggctcctggacttcctccaccttcaaagctgatggagactcccattatgctgctgtaatccccactcccctcttcctcctcctttcatgtggacccctgtgactacactgagcccatcaggacagtccaggctgtctccccatctcaaggtcaactcatcaacaacctgagctccatcttctccttcagtcccttcccctatatcataaatagtcacagactccagggattagaatgtagtcatcactggggacaattattcttcccaccacagcacccatttccctgtattcaatccccctttaccccaaatacagtcaggacttgcatgatgggacccgcaaggacacgcccaccaggagctctgggattcaggaggtgggacaaggagaatcccagacaggagccctctgacctgtgaccgtgatctccagggggttgctgggtgccgaccacccactggggtagtgtggttgtgaaccccgacatgtataggtccctgcgtgtgctggggtcacagggcccatgaaaaggctgttccagaatattatgttgtagagctcagggacaggcaccccatcttccttttacagactgaagttgttaaacccaagataagaatgacactgaagaatcacatgtcctggaggcaccacagggcttggccaggcagacagcaagggcttgtcctgaccaccgtggggagaaggaggcaccgccttagagaggaggatgtggagccgcccctccctccctgtgctctgaagattctcctcgctttccaagtttctatggctgctatcacaccttggtgcccagggctaaaggaaggacccatcccgcaaacacaaggtgtctccctacaacaaaagtgtcagctgagaactttgagcaagtgctgagtaagagactcctactagattttaatactgtaagattactcacataaaacaacacagggtagacatggggtggagggcatgtcctttgagaatggaatatcagccgatgcctgaacgaaaataaacaactgagtccccatcagaggattggaatgtcagggccatggctgtggttttcccacctcttctggtagaatgacagcagccacactgcagcccctaccgtcatggaaacgctgaagtgtgtgagtaacacctttgtcctcagaggatctgctgttcctaccacttccccaccacacaccccagctttgagcaccgtagtctaaccctggtccccacagaacttgactctgccaagggaatgaaaggccagggaggcaaggtcagaaatgtgggcccagcaccccagggtcccttcttcctagtttatgagagactccctgacaggacttccctcccatttcaggaaaatcctcttatgtggggagatgacacccgaaggttgggagaaggactcaccctcatgtggccaggccccctgcagcaagaagaaccctggaaagaaagatcatgatggatgacccatctgcaggcaaaccagggcacccttgctgcccccactgggctgtgagtcttggtagccaggcccttcctgggctgaaggtaaactcaccctcagtgcctacctgcacccaagaacagggctgtcggctgtgcagagacccagcctccaggtccatatccccacctcaagcccatatctccactccaggcccatatctccactccaggccgatatttccaccctaagcccatatcgccaatccaggcccatatctccaatccaggctcagatctccaccctgggcccatatctccaatccaggcccttatctccactccaggtccatatctcctctccagtcccatatctccactccaggcccatatatcctctccagtcccatatctccacacccaggcccgtatctccatcctaggcacatatctcctctccaggcccagatatcgacctctaggcccatatctccactcctggcccatatctccactccaggcccagatatcgacctctaggcccatatctccactcctggcccatatctccactccaggcccatgtctccacttcaggcccatatctctactgcaggcccataactccacctccaggcccatgactccactccaggcccatatctccacctccaggcccatatctcccctccaggttcctatctcccctccaggttcctatctccactccaggcccagatctccactacagtcccatcactccacctccaggcctatatctcgacctctgggcccagatctccacttctaggcccatcactccatctctaggcccatatatccactccaggcccagatctccactccaggcccataactccacctccaggcctatatctccacctctgggcccagatctccatcccctcactccctccctctattgctttccaggactcaccaacacacgccatgctgacgaccaagagcgacatggtgctgccggagcagacaggcagccgcgaccgagctcagctcagcagcgcacaggatgttatttggcgccctgcccatgcagtttacatgttgaccacatcatgggagggtgacgtacgcaggctctttctaccttgcatgaggcccagtgggtgctcgctcaagagcggaacacggcttcctggaaattgttctcgctagaatttgacacctagtgtccttcactatgaccaactcaaaacacgtctgagatccaacctcccgaacacgagatgcctaaaatctgtgctaacatgaaagacttttcatgtatttctattgtttttatctgagattcaaactcttcttcctgtgtaatatgcaaaatatctaataggtattattaatgttttcagagtcattgtcactaataaaccattagaatttttcatgcttgtatttctagtattacagcagaaccagttaaaatgatttaaattcccagggaaggattatgcaattatttacaatcttagaattgtactttatcagtaaaaaccccacctgtaaattctggagttttgtagtttaatctaaaatttgtctcatgacccaagattccagagtcccaactctggagtttgttttccgtctgtctctctccctccctcattttaaattttacagaaatatccagtaacataatgctatagaaaatcaagtttccccagcacgttgggaagccgaggtgggcggatcaactgagataaggagtttgagagcagcctggccaatatagtgaaaccgtgtctctgctaaaaatccaaaaattagccgtgcctggtggcaggcacctgtaacgccagctactcaagaggctgaggcatgagaatcgcttgaacctgggaggcagaagttgcagtgagctgagattgtgtcactgcagtccagcctgggcgacagagcaagactccgcctcaagaaaaaaaagcaaatagcctataataacaaattagagagctctggctactaaatttaaagggttctataaggctacataaagtgcagcatcatcaagagtgtggacacagagagccccttagcagaaacagtgtctaaagtacatccgtgtacacacagtccctttagagttgacaaaggctgccgtgtggtttaaggtggcatagaatgtcttctcaataaataatattaaaccaatgggttatacctaggaaaaaataaatctaactcacactataaaaacacttcttagtttttatctagttgtacattttttatgatttatatttaaatttgagaaataaaagtcatatacggtcatccttcactattcgtgggtgattggtttcgagatctccactcagataccaaaatctgtagatgctcaagcctcttatatgaaatggcacagagtttgcaaataacctatgcacatcctcctgtatacatgaaatcatctctagattacttataattcctgatgcagcctacacacagcttcatttgtgtccattcaacacagttctgctttttgtaactctgtggatactttctctgaatatttttgatttatactcggttcaataaagaactgtaaaccccacagatatggaggagtgactgtatatttatagtgtgaaagatgatgtgttgatatgtgtccctgtgtagatgagactaacaaggcctatgattctacaaatgtttcatcttggaatgactctgccagatttccaggtctgcagagagtaagaatatcacttgttcatgtgattcacgatccttggaacctcctatgtgctacatctttggatggaaataggagtcccagagacaaatgaggctccaccctgcttccagaaactcagagtccgggggtgagaacccagtggagaacagatggggttatgtggacatggtaatgataatggaagtcttaggcaagaaaagagtcccattaccgaaaccatgagggcagacatgtttatttgaaggagggaaaactacattgaaattattttaaaaaatatataagttttactgctgacagaaggctgaaagatactctgaggggaggtggaacagcatgagggaaggtggaacaggacgtgtctaagtgccgtgttaagagggagcctcttgtatgtttggaactgtgagttcctcagtgtgattgcagcctcaagtagactaggaagtaagccagtaaggttggagaggtgggcaggggtcaagtgaaatggagaattgtgggctaagcaaaggagtgtgttttctctccagcaggcagtggggaccttagacatttgtaagcaagagagaggcacattcagatttgtggtgtgaggaagagcgatgccctaagatgcagactcacgccttcagattccagctgctggtacatgggagctggcaacccggttttgagacagggctgttgtctccctagaagatcccctcaaggcctgactgtggtgctcatgggcaggagacaactttggatctggactcagcatttggaagttccgtgtacactctggtatctgttgggggtgtcttgggcctctgagaagggcgagtgatttttctctgtgtgaaaacgcagtgatccaactgtacgtatgtcacctcctgagggtcttgttcatcagagtcctggagagagggaaatcctgagtgagggagggtgctcacgttttccaggactgtttgggaataacactagccacgaggctgggccgaggagcacctacctcgctattcgctgttctgttccctgcaggctcttggtccattacagcagcatgtgtaggagacggaagtcaacaaaagagctcggagggcacttctgggtcctcatttcataagcagataccaacaaacagggggaggccataggtgcctgaggtccctcagttgccaacagcagactcagacattctatctctctgagctcaaggacccatcccatgaatagctctgagttcccatcccattgattctgtctcccactttctgcctgtcatggaaccttctcctggatgtgagtggctgcaggggacatgaggatacagttcagaatcaggcaacggtctgtgagctgaaagcagggacagggagtctggtgccctctctagaaagtcctgcctctgtggctgctgccttgggccagggaccatcctacctgtgaggaacacacacctgagtgctcccatcctgcttccccacatggccctgagctctctggcctctccttcgtgagacttacttttcttgttggagcaccagcgatgaaggagaaagaagaggaggaggatgaagaggatgatgaccactgaggtcccaatcagaacgtgcaggtgtcttgggttacctggaagaagatgagacaccaataagaagctaatcatagcagttcctctttatgaattgtctcgcatttcttgattgacaggtaaccacgtaaaacacctctttaggacaagcacccagatggcgggagacccagctttctcctgctttctcagttatagctctcaaagtaaccatagaatgtgctgaggacacaactactttagttgagatgtttgaccccttcaaacctcacattgaaatttcacccccattgtgggaggttgggcctcttgagaggtgtttgggtcatggaggtggatccatcatgaacagatcaatgctgtcccaaggagacggggttagctagttccccctctattagttcctggagagctggttgttcaaaagaacttggaagctccatcgctccccctcccccttgctccctctcttgccgtgtgatctctgtggtctctgcacagacagaccctccttcccttctgccagagtgggagcagcctgaggccatcacgagaaatagatgctggtgccatgcttccagtacagcctgcagaacggtgaggcaaaccaatctcttttctttagaagttgcccaggctcaagtgttcctttagagcaacaaaaatggactaagacagcaacgtcctgagatcaggaggaacgtcccagagcagcctgggctgtcttcctgttcttcctggaggaggacgtcatgcagtgctttagctgagtgcttcctgtggctccagggtacaaaacccaggctgggctgctttctggcttcccccagctacactgcaaatggggtgactccatatgtcccgagcagcttttctgagccttgagggactggctcacattgaaatgtaggcttctgttttcactcgctgcttatctgttagtaatgaacctgcctatgtaacgtattctctgtgtgttctgtctccctggagtgacggtgagtgataggaattggcgtaggcccaggtgcagtctaggaggtgtttagggtcttttctgggaagactgcactgggattgacacacagcgaatgtgctttaggatttctacatccacagcattcttgagtcaaacaacttgcgttctccaaggaaaggaaacaaaagtgaaatcaagataaaaaagcgaaatagagttatcttatgtccaacagccaggaaatcgtgttgaagcccctgtgaaacgtcctactctttgtgatctcgggagacacatgttaggctgctgttctacctgagaggctgggggaaggaccaccccctccaccatctattgcttcaataccacctgtcctcctgtgaattagtaggaaaggggagcaggagctagtgctggtgctgatctctcattccaagatctggactcactccaaggagtattaatgtttacctccccatggtctatctgaatctccacaggtgattggaagtaggggtgaagtgggggatttgagtgagagggcaagttttttttgtgatgaacagagcactttctctattccacgatctgtgctggaggattcagcaggctttcacattttctatatggtctcatgctcacagaaagccaaatacggaagaggttttaggctcattgcctaatggataagacaaaggatcaaagaagtaattatagagaaatacaaaaatgatgattggaattcaggtgcctttgtcattcgtgtgtgttttattatatttatgcatttcttatttttattttttgagacggagtctccttgtgtcacccaggctggagtgcagtgatgcaatctccactcactgcaacctccacctcctgggttgaagtcgttctcctgcttcatcctcaagagtaggagctgggattacagggatgcaccaccatgctcggctaatttttgtatttttcatagagacagggtttcaccattttggccaggctggtctggaactcctgacttcaagtgatccacccgccttggcctcctgcagtgctgggaattgccttttccacggcctgagcatggggccgtggctgaatgagtcagtgagtcgaagtgtgcgtgcatgagctccgttctctgttaaggcaaagctcttgctctgctgagtcagccagggttgcttcatgaccaacagtaattcattcctgggcaagtggaacttctctaaaacacctcgccctcatcaaatgttccctacccttccctctctcaagcccccaggaatttatcctccagttaggaatgcaggcagaacaaacattgcatttttcctgagaaggatgtcagattgccaatcatttttctagcttgtaggagatctcagctccataaaatgagagattaagagatttcacagagccctgttttgggtccagatccctttcgctgttggagtatctggagtttggagatggtagaagacaggcgtacaatgtcagagctgtgagatgctgagtcaacgcctgaatccaaggtttccacctccccaggtttccaaaagcggatataagagggttctgtactcaccggttttggagcttggttcagtgggtgaaggccaactatttgaagggtttcctagaacatgagacaggagagaggtgaggaaatgagggtgtctgtcctctactcagtggaaatctttgaggttggttcatggccaacactctgttatctaatattgggccctgggagtcctgggatccttttttccgtaatttttgtatgtgacggctactgtcttgagacttcaaggtataaagagaaaacaggagcatcacactacctgatctcaaaatatgttacagagctgtagtaagcaagacagcatgacgttggcatgaagaaaggcacatagaacaacggagcagaatgaataacacagatataatccatgcatttacctccaatgtattttttgtttttcttttgagatggagtcttgctctgtcacccaggctggagtgcagaggtgcaatctcggttcactgccaccacagcctcctgggttcaatcacttctcttgcctcaaactcctgagtagtggtattacaggtgctgaccaccatgctcagctaatttttatatttttagtggagacgatgtttcatcacgttggccagactaatcttgaactcttggcctcaggtgatccacccacctcgggctcccaaagtgctgaaattgcaggtgtcagccaccatgcccagcccatccaatggactttgacaaaggtgccaagaactcacaatcaggaaaggacagtcttttcaataaacagtgcagggaaacctggacatcgacatgcagaggaatgaaactgcacctctgcctgtcactatacacaaaaatcaaatgaaaatggattaaagatgtgagtctaaggcctgaacctatgaaacacgtagaagaaaatattggggaaatgctccaggacgtttgtctgaaggaagacattttgttttaaaccttcaaaacacaagtaatcgaagcaaaaatagaccattgggattacctcaaactaagcaacttctgcaccgctaaaaataaaccaacaaagtgaagagacaacccacagattgggagcaaatatgtgcaaactatgcatctgagatgggattaataactagaaatataagaagctcaaacaactcaataaaacaaatgatttaattgaaacaggagcaaaagacatgaaatttccccacatacgaaaaagtgctcagtatcactcatcatcagagaaacacaaattaaaatcaaagtgagttttcatctcaccccattaaaatggcttttaggccgggcgtggtggctcacgtctgtcatcctagaactttgagagcctgaggtgggtgaatctcataaggtcgggagtttgagaccagtctgacccacatggagaaacactgtctctactaaaaatacaaaaattagtcgggcgtggtggcgtgtgcctgtaattccagctactcgggaggctgaggcaggagaatcgcttgaacctgggaggtggaggttgtggtgagccgagatcgcaccactgcactcagcctgggtgacaagagcgaaactccatctcaaaataaaatgaaataaaataaaatggcttttagctgcaagacaggcaaaagaaatgctggcaaggtgttagagaaaggagaatcctggtatcctgttggtaggagtgtaaattagtacagccattacggagaaaagtgtggaagtcctttaaagaactaaaaagaggttgggtgaggtggatcatgcctgtaatcccggcactttgggagaccgaggcgggcacctcagttgaggtcatgagtttgagagcagcccagccaacatggggaaaccgcatctatactaaaaaaaacaaaaagtagccaggcatggtggcgtgcgcctataatccctgatactagggaggctgaggcaggaaaatcatttgaacccaggaggcagaggttgcaatgagccaagatgacatcacttgtactccagcctgggcacagagggaaactgtctcaaaaacaaaaacaaaacaacaaacgaaaaactaaaaagagaactttcatagtatccagcaatttcactactgggtttatatccaaaggaaagtaaatcaatatatcgaagtgatatctgcactcgtatgattggtgcagcactcttcacagtagccaagatgaggagtcaacctacctgcccatcagtgggtgaatggatagagagaatgtggtacatttgcatagtggagactactcttccatagaaagaaaaacttcctgatatttgcagccacatggatggaactggaggtcattacaaagattcccatttcttacccatataca

SEQ ID NO: 40 - DNA, 6375 bp, synthetic construct
  source 1..6375: mol_type = other DNA
  misc_feature 1..6375: note = HERV
tgggtgtcgggggtgcacggcaacatattcaagcttatgtacaaggcgtttgaggtcggggcatggaaacatactgaggcactgtgtgtatgttatttgtgcatgagaatgaaactccttgaccctgaaaacaggacggggagtggagtgtgtggtgtgataaggaacgctgaaaacagcctcccgagaatgcggtttgagtgcttttaccaggccacaggtgtctcatgacccgacctcaaaaaggccatctagtggatgtttgtggtctaacaagccctttcaatgaatacttggcggacagatgctggggcggagtctcttagaagagctgcccccggcccccctcagctggatttgtctgagaactcattcttggcgttcactgcaagctataagctctgcaagtggtgaccccgacgtgatcgccttgaagttacgcgtgaaggaggagagctcattacttttcggggaatcctggtaagggacagtcctgactcccatcaggaggacgggacccatgcgtaaactatcgcgggttgggttatcatgggtcaggaaatgaccaaagaacagaaagtattttttaaaacagtgcaacagctacttaaggctatccagtgcactgtagagcctggagctctacacaagcttatgcttttaatttggcagaaatgcccttggtttcctgatcaaggaaccttagatttagggttatgagagcaggtaggtcgctgcctgaaaagaggatatgagcagggtcattttactaatgttactattttgaccacctgggcgctggtacgctctgcgttgtatcctctttatctgccagatcgtggttagatcgcccattatctccacctgaaaagaattcagaagatttgaagggagagattcctctccccacttcattccgttctatggggaataaggaagaggtttttttctagtgaggacaaacaggaaccagaacagtggttggggggtgggggtgctcctctctctacctcattcccttctgtagggcataaggaggannnnnnnnnnnnnnnnnnnnnnnagtgaggataaggacggacgggagccttttcctccccctgtagaaaagccattgcccttctttcctccacccttaaaaggacctgcatttgttggccctgttcagccaacagcgccttccatcccccttaaggagattggaggccgcccaagggacggctcttagataagacccccggtcagcgaacatctgtatgatctgtatgattgggtggctgcgtctcctcgactaacttccctgctggaggggtgcgtccaggagggaaggaaagtggctgattatgattgtcttcctaatatgcaagttcccatttgctacttccagcatcagcctttctggccttgtcttttttctgtttcactggagtaaaaggggaagttgcatgctgcctcctgggttttatcccagatagctctagctttcttgctgcccacagaggcctggggcaggagagttgctgagatgccatggagtgcacacttggtcactggcagcctgggcaggttgcccctttctgggtttgtggtgacggagggaaggccaaaaggcacagaccgagtccccgggtggctgcaggcagctccagcccagtcctgaggatccgcctcaccatggtcacgtgccttagtaactgtgcccaggaagtggcctgctgcttgccgtgctgctgcttttcctacttctgcccttccctgccactcctcacatgtctcagttgacgagcaattccttgtcttccctggccccctagggaaagggctgagaaatagtccatgtgcacccggaccttactagcctaaggtgggcaaaggagtgtggagcagcccggagtacagagccctggaggaggagcccactaataaggggcgctctcccatagccatatattaaatgctaactagactgagggggacgagctctgccagctgctgtcatcttcagaagatagacgcagcagtaaggagtgtttgttttgcttttttacaaaatgtttaaaaacactgtggttaagaaacttcaaagcagaccctgtgctgcatgtctgctcctcccctgagcctctctgcttgggggtggtaaaaataataaaaagcccagtatattttcagtacctcacctaacagggttggctgcaggcgtaggtggcctagaagataaggggagtgtttttctcccagcctgttaccttcttgcctccagcctccgcacttccacacacagtttaccacccggtcattctctggcctcttactgccgcttgtactcttccttccttcctctcagggtaaggacagcaacaagtggcaggctattaaaaagagaagctgcctaaggggcccagaacgaatagaaagaaactggccacagccctactttcccaagctcacttctaagacattccagctaaaggctgatgcaggaaaatggctaacaccaaaggacattttaaaaagcttttttttgttttcttttctttttaacacacttaagctaactcaatgcaggtttattatcctggcgacttgcagtcactttctaatggctttcaagggccaaaatatagtaaaaatcacttaaaatatccatcctttccatgccttagtttaacaggtaggctttatcttttgccatttcggtattttatatgttatgtacctgtctcataatccctctaaacttatacaaaagactacagatataatagcttctcttctcataaaaggacgccaacgttgcgtccagctgtcaggataagaccctgctactcttttcctacctttaagcaaggaacaatttcacactctcttagcttgtgatcttgattggcaagtagcaatggctgtctttattggtaatatcagctttcatttaccagcctctaagctcctgaactttttacaaactgtacctgtttaatttgtatgtattgttgtctctgagcctttacttcatgccaccactgtctttacagatggttaaaaaaaaacccaaaaactgaaaaagcagctatagtgtggcaagatgccatgcagaactggcagtacaaaatccaggagcattttaaaaactatgcaacaggcggaattaggtgccctgatattggccttacaaacttttcctcaccaagacataaatatcgttggtgattccgcttatgtggtgtatagtattactcatttagatcttgcacatgtgaagggcattactaataaacccctattagctttgtttcttgcagcgcaagagctcctctgtgcctgtcatcaccctctttacatcacacatattcgccgtcattctgggctacctggtccttatcagaagggaatgcttgagctgatgctctggtacgaccacagatgtgctttgcaaattctcctgcttttttgcaagctcaagctgatcatgttgttttttcatcggaacgcccgccgtcttaaacaacagcttcatttgacacttactcaagcttgcatgattattaaaacttgtcctaattgccaacagcattctctttcccccttttccttagggcttagtgccaacctacgaggtctggtgcctaatgctatctggcaaactaatgtcactcggtgtccaccctttggacgttttaaatttctccatgttaccgtggacacatatacaggcctaatacatgctacctcccagacaggagaaaaaactaaagatgcaatcgctcatttgtttaaatctatgataactctaagccttccacacactataaaaactaataatggaccttcctatcttagtgctcgatttacatatgcattgcaactttggcacatgcaacataaaactgatattccttataactcaaccagtcaggccattgttaaacgagctcatcaaactcttaaagtatatcttaatttaaaaagggcgggggggaatatggggctctcttctagagaacaggaccagtcgttggagaaccaaccaacaatgatggtgatgactcggaaggcacctgacatcacctggggacagctaaagaaattgaatcaacaagcatctatccagcttgccgccgtggaagcccctgcaactgcagacaatcggtttcttacatatctggtggcaattggggaaacttctgagaaagtaagacagacatggatgttggggtggcaggtaattcttgtcctttgctaagtgggatcagcttaaggacatgtttattggagtcatgttctaaatccccctgtttttaatgttattacatggtgggatgctgacccgcctttgtcatctaatgatacttcttgggcaggaggccgatggatgcctctgtcttaccccgtaactgaaaatttgggatggattcaacttaattactcctcgattttattgtctagtaatccccctctttgtttttccacaatagcacatgataagtgtgttactctcatccctcaggagtatctttactatcagcctaaaagggatgctaagcttgcaaacttgacctttatttctactattaccactaatcttactgaggtctctaatgaagctacacaagtgcctgatcttcctatatgccgtccgagtagggactggcgataaaagtttgaggccgtccagtggtcgccgtgcagacagcctgctccacgtcaagggcctctgtttgataatggcaccttacttgattggggtccccgtggtaatcttatgtctgcaaatcagactattggacttgggagtccctccagtagtcctattacctagtcagagtatgggctttcaggaccagtattacaagtgagagggaaacaagctttaagccctgctcatacccaaatttggagattaggatttcctttttttgaacgggttctttcacatggtgagtatattactggcggtggcaaccataccctctctttgcataataatgttactgacacagtcctgatttgtaccacgcacccttatatacttttgtttgggcaaggtgttcctaacatagagcagaatcaatctttttatagtattaaagtctcttccagtagttggtatgctgcatgcttgtccatcgaaacattacacagttggatataacctatgtcatgatcttaaaatggcgtgcagaattgtggctgcctgtaaatttaacctggagctgaaaaggagattccaccctgcagctatttagaaaatcactatctcatactcggnnnnnnnnnnnnnnnnnnngattcctggccactttaattgcctttttagtctcagctattattatattagcaactgctgctactgcagctgtttctctgacagaatctattcacacagcctcagtggtgaaccacatggtgtataatgtaacccgtgaatttcaagaacaggtaaatatagataaaaccattctgtctcgcccggtctcttgaagccgctgttgaataactggggaatcagcagcaggcattcattacccatcaaaatttacattgttgattggcaatataattctatctgtgtcacgcctctgccatataatagctcccaatatgcttgggagagagtgaaagcacatctgcaaggggcttatcacgaccatttgtcttctcaaatttccattcttgagtgtgaattaaagaaacaccttgaagaatggtcgcagcaattacaaacaagtatccttcagcaattacaagaaggctttcaatggttaaacccgaacacttggttgtctgggttaaacatacgcatttgggtgatggccgctgtacttattcttttttgtatctgtttgctaggcacttgcagatggctctgtgctgccacccgacgcatctatgaccagggaagaatcatggaagcttaccttgcattggatgaccagcacgctataagaattaaagaaggggggaatatggcggggtgcacgacagcatattcaagcttatgtacaaggcgtttgaggtcggggcatggaaacatactgaggcactgtgtgtatgttatttgtgcatgagaatgaaactccttgaccctgaaaacaggacggggagtggagtgtgtggtgtgataaggaacgctgaaaacagcctcccgagaatgcggtttgagtgcttttaccaggccacaggtgtctcatgacccgacctcaaaaaggccatctagtggatgtttgtggtctaacaagccctttcaatgaatacttggcggacagatgctggggcggagtctcttagaagagctgcccccggcccccctcagctggatttgtctgagaactcattcttggcgttcactgcaagctataagctctgcgatgggcc

SEQ ID NO: 41 - DNA, 6374 bp, synthetic construct
  source 1..6374: mol_type = other DNA
  misc_feature 1..6374: note = HERV
tgggtgtcgggggtgcacggcaacatattcaagcttatgtacaaggcgtttgaggtcggggcatggaaacatactgaggcactgtgtgtatgttatttgtgcatgagaatgaaactccttgaccctgaaaacaggacggggagtggagtgtgtggtgtgataaggaacgctgaaaacagcctcccgagaatgcggtttgagtgcttttaccaggccacaggtgtctcatgacccgacctcaaaaaggccatctagtggatgtttgtggtctaacaagccctttcaatgaatacttggcggacagatgctggggcggagtctcttagaagagctgcccccggcccccctcagctggatttgtctgagaactcattcttggcgttcactgcaagctataagctctgcaagtggtgaccccgacgtgatcgccttgaagttacgcgtgaaggaggagagctcattacttttcggggaatcctggtaagggacagtcctgactcccatcaggaggacgggacccatgcgtaaactatcgcgggttgggttatcatgggtcaggaaatgaccaaagaacagaaagtattttttaaaacagtgcaacagctacttaaggctatccagtgcactgtagagcctggagctctacacaagcttatgcttttaatttggcagaaatgcccttggtttcctgatcaaggaaccttagatttagggttatgagagcaggtaggtcgctgcctgaaaagaggatatgagcagggtcattttactaatgttactattttgaccacctgggcgctggtacgctctgcgttgtatcctctttatctgccagatcgtggttagatcgcccattatctccacctgaaaagaattcagaagatttgaagggagagattcctctccccacttcattccgttctatggggaataaggaagaggtttttttctagtgaggacaaacaggaaccagaacagtggttggggggtgggggtgctcctctctctacctcattcccttctgtagggcataaggaggannnnnnnnnnnnnnnnnnnnnnnagtgaggataaggacggacgggagccttttcctccccctgtagaaaagccattgcccttctttcctccacccttaaaaggacctgcatttgttggccctgttcagccaacagcgccttccatcccccttaaggagattggaggccgcccaagggacggctcttagataagacccccggtcagcgaacatctgtatgatctgtatgattgggtggctgcgtctcctcgactaacttccctgctggaggggtgcgtccaggagggaaggaaagtggctgattatgattgtcttcctaatatgcaagttcccatttgctacttccagcatcagcctttctggccttgtcttttttctgtttcactggagtaaaaggggaagttgcatgctgcctcctgggttttatcccagatagctctagctttcttgctgcccacagaggcctggggcaggagagttgctgagatgccatggagtgcacacttggtcactggcagcctgggcaggttgcccctttctgggtttgtggtgacggagggaaggccaaaaggcacagaccgagtccccgggtggctgcaggcagctccagcccagtcctgaggatccgcctcaccatggtcacgtgccttagtaactgtgcccaggaagtggcctgctgcttgccgtgctgctgcttttcctacttctgcccttccctgccactcctcacatgtctcagttgacgagcaattccttgtcttccctggccccctagggaaagggctgagaaatagtccatgtgcacccggaccttactagcctaaggtgggcaaaggagtgtggagcagcccggagtacagagccctggaggaggagcccactaataaggggcgctctcccatagccatatattaaatgctaactagactgagggggacgagctctgccagctgctgtcatcttcagaagatagacgcagcagtaaggagtgtttgttttgcttttttacaaaatgtttaaaaacactgtggttaagaaacttcaaagcagaccctgtgctgcatgtctgctcctcccctgagcctctctgcttgggggtggtaaaaataataaaaagcccagtatattttcagtacctcacctaacagggttggctgcaggcgtaggtggcctagaagataaggggagtgtttttctcccagcctgttaccttcttgcctccagcctccgcacttccacacacagtttaccacccggtcattctctggcctcttactgccgcttgtactcttccttccttcctctcagggtaaggacagcaacaagtggcaggctattaaaaagagaagctgcctaaggggcccagaacgaatagaaagaaactggccacagccctactttcccaagctcacttctaagacattccagctaaaggctgatgcaggaaaatggctaacaccaaaggacattttaaaaagcttttttttgttttcttttctttttaacacacttaagctaactcaatgcaggtttattatcctggcgacttgcagtcactttctaatggctttcaagggccaaaatatagtaaaaatcacttaaaatatccatcctttccatgccttagtttaacaggtaggctttatcttttgccatttcggtattttatatgttatgtacctgtctcataatccctctaaacttatacaaaagactacagatataatagcttctcttctcataaaaggacgccaacgttgcgtccagctgtcaggataagaccctgctactcttttcctacctttaagcaaggaacaatttcacactctcttagcttgtgatcttgattggcaagtagcaatggctgtctttattggtaatatcagctttcatttaccagcctctaagctcctgaactttttacaaactgtacctgtttaatttgtatgtattgttgtctctgagcctttacttcatgccaccactgtctttacagatggttaaaaaaaaacccaaaaactgaaaaagcagctatagtgtggcaagatgccatgcagaactggcagtacaaaatccaggagcattttaaaaactatgcaacaggcggaattaggtgccctgatattggccttacaaacttttcctcaccaagacataaatatcgttggtgattccgcttatgtggtgtatagtattactcatttagatcttgcacatgtgaagggcattactaataaacccctattagctttgtttcttgcagcgcaagagctcctctgtgcctgtcatcaccctctttacatcacacatattcgccgtcattctgggctacctggtccttatcagaagggaatgcttgagctgatgctctggtacgaccacagatgtgctttgcaaattctcctgcttttttgcaagctcaagctgatcatgttgttttttcatcggaacgcccgccgtcttaaacaacagcttcatttgacacttactcaagcttgcatgattattaaaacttgtcctaattgccaacagcattctctttcccccttttccttagggcttagtgccaacctacgaggtctggtgcctaatgctatctggcaaactaatgtcactcggtgtccaccctttggacgttttaaatttctccatgttaccgtggacacatatacaggcctaatacatgctacctcccagacaggagaaaaaactaaagatgcaatcgctcatttgtttaaatctatgataactctaagccttccacacactataaaaactaataatggaccttcctatcttagtgctcgatttacatatgcattgcaactttggcacatgcaacataaaactgatattccttataactcaaccagtcaggccattgttaaacgagctcatcaaactcttaaagtatatcttaatttaaaaagggcgggggggaatatggggctctcttctagagaacaggaccagtcgttggagaaccaaccaacaatgatggtgatgactcggaaggcacctgacatcacctggggacagctaaagaaattgaatcaacaagcatctatccagcttgccgccgtggaagcccctgcaactgcagacaatcggtttcttacatatctggtggcaattggggaaacttctgagaaagtaagacagacatggatgttggggtggcaggtaattcttgtcctttgctaagtgggatcagcttaaggacatgtttattggagtcatgttctaaatccccctgtttttaatgttattacatggtgggatgctgacccgcctttgtcatctaatgatacttcttgggcaggaggccgatggatgcctctgtcttaccccgtaactgaaaatttgggatggattcaacttaattactcctcgattttattgtctagtaatccccctctttgtttttccacaatagcacatgataagtgtgttactctcatccctcaggagtatctttactatcagcctaaaagggatgctaagcttgcaaacttgacctttatttctactattaccactaatcttactgaggtctctaatgaagctacacaagtgcctgatcttcctatatgccgtccgagtagggactggcgataaaagtttgaggccgtccagtggtcgccgtgcagacagcctgctccacgtcaagggcctctgtttgataatggcaccttacttgattggggtccccgtggtaatcttatgtctgcaaatcagactattggacttgggagtccctccagtagtcctattacctagtcagagtatgggctttcaggaccagtattacaagtgagagggaaacaagctttaagccctgctcatacccaaatttggagattaggatttcctttttttgaacgggttctttcacatggtgagtatattactggcggtggcaaccataccctctctttgcataataatgttactgacacagtcctgatttgtaccacgcacccttatatacttttgtttgggcaaggtgttcctaacatagagcagaatcaatctttttatagtattaaagtctcttccagtagttggtatgctgcatgcttgtccatcgaaacattacacagttggatataacctatgtcatgatcttaaaatggcgtgcagaattgtggctgcctgtaaatttaacctggagctgaaaaggagattccaccctgcagctatttagaaaatcactatctcatactcggnnnnnnnnnnnnnnnnnngattcctggccactttaattgcctttttagtctcagctattattatattagcaactgctgctactgcagctgtttctctgacagaatctattcacacagcctcagtggtgaaccacatggtgtataatgtaacccgtgaatttcaagaacaggtaaatatagataaaaccattctgtctcgcccggtctcttgaagccgctgttgaataactggggaatcagcagcaggcattcattacccatcaaaatttacattgttgattggcaatataattctatctgtgtcacgcctctgccatataatagctcccaatatgcttgggagagagtgaaagcacatctgcaaggggcttatcacgaccatttgtcttctcaaatttccattcttgagtgtgaattaaagaaacaccttgaagaatggtcgcagcaattacaaacaagtatccttcagcaattacaagaaggctttcaatggttaaacccgaacacttggttgtctgggttaaacatacgcatttgggtgatggccgctgtacttattcttttttgtatctgtttgctaggcacttgcagatggctctgtgctgccacccgacgcatctatgaccagggaagaatcatggaagcttaccttgcattggatgaccagcacgctataagaattaaagaaggggggaatatggcggggtgcacgacagcatattcaagcttatgtacaaggcgtttgaggtcggggcatggaaacatactgaggcactgtgtgtatgttatttgtgcatgagaatgaaactccttgaccctgaaaacaggacggggagtggagtgtgtggtgtgataaggaacgctgaaaacagcctcccgagaatgcggtttgagtgcttttaccaggccacaggtgtctcatgacccgacctcaaaaaggccatctagtggatgtttgtggtctaacaagccctttcaatgaatacttggcggacagatgctggggcggagtctcttagaagagctgcccccggcccccctcagctggatttgtctgagaactcattcttggcgttcactgcaagctataagctctgcgatgggcc

(19) United States
(12) Patent Application Publication
(10) Pub. No.: US 2025/0147046 A1
(43) Pub. Date: May 8, 2025

(54) NEW METHOD FOR IDENTIFYING HERV-DERIVED EPITOPES

(71) Applicants: ERVIMMUNE, Lyon Cedex 08 (FR); CENTRE LEON BERARD, Lyon (FR); UNIVERSITE CLAUDE BERNARD LYON 1, Villeurbanne (FR); CENTRE NATIONAL DE LA RECHERCHE SCIENTIFIQUE, Paris (FR); INSTITUT NATIONAL DE LA SANTE ET DE LA RECHERCHE MEDICALE (INSERM), Paris (FR)

(72) Inventors: Stéphane DEPIL, Lyon (FR); Vincent ALCAZER, Pierre-Benite (FR)

(73) Assignees: ERVIMMUNE, Lyon Cedex 08 (FR); CENTRE LEON BERARD, Lyon (FR); UNIVERSITE CLAUDE BERNARD LYON 1, Villeurbanne (FR); CENTRE NATIONAL DE LA RECHERCHE SCIENTIFIQUE, Paris (FR); INSTITUT NATIONAL DE LA SANTE ET DE LA RECHERCHE MEDICALE (INSERM), Paris (FR)

(21) Appl. No.: 18/832,712

(22) PCT Filed: Jan. 25, 2023
(86) PCT No.: PCT/EP2023/051839

§ 371 Date: Jul. 24, 2024

(30) Foreign Application Priority Data
Jan. 25, 2022 (EP) 22305079.0

(51) Int. Cl. G01N 33/68; A61K 31/7088; A61K 35/17; A61K 38/16; A61P 35/00; C07K 14/15; C12N 5/0783; G16B 30/10

(52) U.S. Cl. CPC G01N 33/6893; A61K 31/7088; A61K 35/17; A61K 38/162; A61P 35/00; C07K 14/15; C12N 5/0638; G16B 30/10; C12N 2510/00

(57) ABSTRACT

Methods for identifying HERV-derived T cell epitopes associated with cancer, and peptides that are or that include epitopes identified by the methods, expression vectors encoding the peptides, cytotoxic T lymphocytes (CTLs) of a subject treated with the peptides or vectors and engineered T cells expressing T-cell receptors recognizing the peptides. Also, the peptides, expression vectors, CTLs or engineered T cells as a vaccine or a medicament, and in particular the use of the peptides, expression vectors, CTLs or engineered T cells for use in preventing or treating cancer in a subject in need thereof.

### FIELD OF INVENTION

The present invention relates to methods for identifying HERV-derived T cell epitopes associated with cancer, peptides comprising or consisting of the epitopes identified by said method, expression vectors encoding said peptides, cytotoxic T lymphocytes (CTLs) of a subject treated with said peptides or vectors and engineered T cells expressing T-cell receptors recognizing said peptides. The present invention also relates to the use of said peptides, expression vectors, CTLs or engineered T cells as a vaccine or a medicament, and in particular, the use of said peptides, expression vectors, CTLs, or engineered T cells for preventing or treating a cancer in a subject in need thereof.

### BACKGROUND OF INVENTION

The adaptive T cell immune response in cancer relies on the recognition of tumor epitopes specifically expressed by tumor cells. The role of neoantigens, generated by non-synonymous mutations specific to the tumor genome, has been extensively studied in the last decade and many clinical trials testing combinations of neoantigens in personalized cancer vaccines have been initiated, with encouraging preliminary results. However, determining the optimal combination of neoepitopes for each patient remains challenging. Furthermore, many tumors are characterized by a low or moderate tumor mutational burden. Therefore, unveiling other families of tumor antigens, such as those derived from splice variants, fusion proteins or endogenous retroelements, possibly shared among different cancer subtypes, is of utmost importance for the development of off-the-shelf therapies in solid tumors.

Human endogenous retroviruses (HERVs) represent 8% of the human genome. Although most HERV genes are non-functional due to DNA recombination, mutations, and deletions, some produce functional proteins including the group-specific antigen (Gag), polymerase (Pol) with reverse transcriptase, and the envelope (Env) surface unit. Most HERVs are silenced by epigenetic mechanisms in normal cells. However, HERVs were reported to be possible pathogenic agents in carcinogenesis, through their involvement in insertional mutagenesis, chromosomal aberrations or LTR-induced oncogene-activation. Thus, HERVs may represent an interesting source of shared tumor antigens.

Among the prior art, the patent application WO2020/049169 describes a method that enabled the identification of epitopes derived from HERVs and able to elicit a specific CD8+ T cell response. However, the method developed in this patent application was applied to a single cancer type, i.e. triple-negative breast cancer (TNBC), having a limited number of HERVs. Thus, there is still a need to develop new approaches able to identify HERVs-derived antigens that are efficient to induce CD8+ T cells response and that are shared among multiple cancer subtypes.

In the present application, the Inventors have developed a new approach that addresses this need. In particular, the Inventors have developed a method relying on selection steps that allows the determination of a limited number of epitopes, that are efficient and shared by multiple cancer subtypes, among a large number of HERV candidates.

### SUMMARY

The present invention relates to a method for identifying Human endogenous retroviruses (HERVs)-derived T cells epitopes associated with at least one cancer, wherein said method comprises the following steps:
(a). Identifying HERVs associated with at least one cancer, and
(b). Selecting T cell epitopes among the HER Vs identified in the previous step, and
wherein said method further comprises at least one, preferably two, of the following steps:
   (i). Selecting HERVs associated with a cytotoxic T cells response among the cancer-associated HERVs identified in step (a), said step being between step (a) and step (b), and/or
   (ii). Assessing the expression at the protein or peptide level of the HERVs-derived T cells epitopes identified in step (b) in tumor samples, said step being after step (b).

In one embodiment, steps (a) and (b) and steps (i) and/or (ii) are in silico steps. In one embodiment, step (ii) is an in vitro step.

The present invention relates to an in silico method for identifying Human endogenous retroviruses (HERVs)-derived T cells epitopes associated with a cancer, wherein said method comprises the following steps:
(a) Identifying and selecting HERVs associated with a cancer, and
(b) Selecting shared T cell epitopes among the HERVs identified in the previous step, and wherein said method further comprises at least one, preferably two, of the following steps:
   (i). Selecting HERVs associated with a cytotoxic T cells response among the cancer-associated HERVs identified in step (a), said step being between the step (a) and the step (b), and/or
   (ii). Assessing the expression at the protein or peptide level of the HERVs-derived T cells epitopes identified in step (b) in tumor samples, said step being after the step (b).

The present invention relates to an in silico method for identifying Human endogenous retroviruses (HERVs)-derived T cells epitopes associated with a cancer, preferably associated with at least one cancer, wherein said method comprises the following steps:
(a) Identifying HERVs associated with a cancer, preferably associated with at least one cancer, and
(b) Selecting T cell epitopes among the HERVs identified in the previous step, and
   wherein said method further comprises at least one, preferably two, of the following steps:
(i). Selecting HERVs associated with a cytotoxic T cells response among the cancer-associated HERVs identified in step (a), said step being between the step (a) and the step (b), and/or
(ii). Assessing the expression at the protein or peptide level of the HERVs-derived T cells epitopes identified in step (b) in tumor samples, said step being after the step (b).

In one embodiment, the step (a) comprises the step of comparing HERVs expression in tumor and in normal samples.

In one embodiment, the step (b) comprises the step of aligning the sequences of the HERVs identified in the previous step with HERV proteins.

In one embodiment, the step (b) further comprises the step of predicting the binding of the sequences sharing at least 70, 75, 80, 85, 90, 95, 96, 97, 98, 99% or more identity with HERV proteins to MHC class I molecules.

In one embodiment, the association of the cancer-associated HERVs with a cytotoxic T cells response in the step (i) is assessed by the association of each HERV with at least one CD4 or CD8 T cell signature, the association of each HERV with a function signature being either interferon (IFN)-γ signature or cytolytic activity, and the absence of expression of each HERV in normal purified T or NK cells, preferably said association is assessed by a machine learning-based approach.

In one embodiment, said method further comprises, after the step (b) or before the step (ii), a step of selecting epitopes among the most shared epitopes in the cancer-associated HERVs identified in step (a).

In one embodiment, said method further comprises, after the step (b) or after the step (ii), a step of aligning the HERVs-derived T cell epitopes with human proteome.

The present invention also relates to a peptide comprising or consisting of an epitope identified by the method as described previously.

The present invention also relates to a peptide comprising or consisting of an epitope having a sequence selected in the group comprising or consisting of RMLTDLRAV (SEQ ID NO: 3), LMAQAITGV (SEQ ID NO: 11), VLQDFDQPI (SEQ ID NO: 13), ALMIVSMVV (SEQ ID NO: 14), MLLAALMIV (SEQ ID NO: 15) and YIDDILCAA (SEQ ID NO: 16). The present invention also relates to a peptide comprising or consisting of a sequence selected in the group comprising or consisting of RMLTDLRAV (SEQ ID NO: 3), LMAQAITGV (SEQ ID NO: 11), VLQDFDQPI (SEQ ID NO: 13), ALMIVSMVV (SEQ ID NO: 14), MLLAALMIV (SEQ ID NO: 15) and YIDDILCAA (SEQ ID NO: 16).

The present invention also relates to a peptide comprising or consisting of an epitope having a sequence selected in the group comprising or consisting of RMLTDLRAV (SEQ ID NO: 3), LMAQAITGV (SEQ ID NO: 11), VLQDFDQPI (SEQ ID NO: 13), MLLAALMIV (SEQ ID NO: 15) and YIDDILCAA (SEQ ID NO: 16). The present invention also relates to a peptide comprising or consisting of a sequence selected in the group comprising or consisting of RMLTDLRAV (SEQ ID NO: 3), LMAQAITGV (SEQ ID NO: 11), VLQDFDQPI (SEQ ID NO: 13), MLLAALMIV (SEQ ID NO: 15) and YIDDILCAA (SEQ ID NO: 16).

The present invention also relates to an expression vector inducing expression of one or more peptide(s) as described hereinabove.

The present invention also relates to a cytotoxic T-lymphocyte of a subject treated with one or more peptide(s), or one or more expression vector(s) as described hereinabove.

The present invention also relates to an engineered T cell expressing a T-cell receptor recognizing a peptide as described hereinabove.

The present invention also relates to one or more peptide(s), one or more expression vector(s), one or more cytotoxic T-lymphocyte(s), or one or more engineered T cell(s) as described hereinabove for use as a vaccine or medicament.

The present invention also relates to one or more peptide(s), one or more expression vector(s), one or more cytotoxic T-lymphocyte(s), or one or more engineered T cell(s) as described hereinabove for use in preventing or treating a cancer in a subject in need thereof.

In one embodiment, said cancer is selected from the group comprising or consisting of breast cancer, including triple negative breast cancer, ovarian cancer, melanoma, sarcoma, teratocarcinoma, bladder cancer, lung cancer, including non-small cell lung carcinoma and small cell lung carcinoma, head and neck cancer, colorectal cancer, glioblastoma, leukemias, lymphomas and other solid tumors and hematological malignancies.

### Definitions

In the present invention, the following terms have the following meanings:

“Epitope” refers to a portion of an antigen, that is capable of stimulating an immune response.

“Human endogenous retroviruses (HERV)”: refers to retroviruses that have integrated into the germline that have lost infectious capability but retained the capability to transpose.

“Peptide” refers to a linear polymer of amino acids of at least 2 amino acids linked together by peptide bonds. Amino acid residues in peptides are abbreviated as follows: Phenylalanine is Phe or F; Leucine is Leu or L; Isoleucine is Ile or I; Methionine is Met or M; Valine is Val or V; Serine is Ser or S; Proline is Pro or P; Threonine is Thr or T; Alanine is Ala or A; Tyrosine is Tyr or Y; Histidine is His or H; Glutamine is Gln or Q; Asparagine is Asn or N; Lysine is Lys or K; Aspartic Acid is Asp or D; Glutamic Acid is Glu or E; Cysteine is Cys or C; Tryptophan is Trp or W; Arginine is Arg or R; and Glycine is Gly or G.

“Prevent”, “preventing” and “prevention” refer to prophylactic and preventative measures, wherein the object is to reduce the chances that a subject will develop the pathologic condition or disorder over a given period of time. Such a reduction may be reflected, e.g., in a delayed onset of at least one symptom of the pathologic condition or disorder in the subject.

“Protective immune response” refers to a cytotoxic T lymphocytes (CTL) and/or an helper T lymphocytes (HTL) response to an antigen derived from an infectious agent or a tumor antigen, which prevents or at least partially arrests disease symptoms or progression. The immune response may also include an antibody response which has been facilitated by the stimulation of helper T cells.

“Subject” refers to a mammal, preferably a human. In one embodiment, a subject may be a “patient”, i.e., a warm-blooded animal, more preferably a human, who/which is awaiting the receipt of, or is receiving medical care or was/is/will be the object of a medical procedure, or is monitored for the development of a disease. The term “mammal” refers here to any mammal, including humans, domestic and farm animals, and zoo, sports, or pet animals, such as dogs, cats, cattle, horses, sheep, pigs, goats, rabbits, etc. Preferably, the mammal is a primate, more preferably a human.

“Therapeutically effective amount” refers to the level or amount of one or more peptide(s), one or more expression vector(s), one or more CTL(s), or one or more engineered T cell(s) as described herein that is aimed at, without causing significant negative or adverse side effects to the target, (1) delaying or preventing the onset of a disease, disorder, or condition; (2) slowing down or stopping the progression, aggravation, or deterioration of one or more symptoms of the disease, disorder, or condition; (3) bringing about ameliorations of the symptoms of the disease, disorder, or condition; (4) reducing the severity or incidence of the disease, disorder, or condition; or (5) curing the disease, disorder, or condition. A therapeutically effective amount may be administered prior to the onset of the disease, disorder, or condition, for a prophylactic or preventive action. Alternatively or additionally, the therapeutically effective amount may be administered after initiation of the disease, disorder, or condition, for a therapeutic action.

“Treating” or “treatment” or “alleviation” refers to therapeutic treatment; wherein the object is to slow down (lessen) the targeted pathologic condition or disorder. A subject or mammal is successfully “treated” for a cancer if, after receiving a therapeutic amount of the one or more peptide(s), one or more expression vector(s), one or more cytotoxic T lymphocyte(s) or one or more engineered T cell(s) according to the present invention, the patient shows observable and/or measurable reduction in or absence of one or more of the following: reduction in the number of cancer cells (or tumor size); reduction in the percent of total cells that are cancerous; and/or relief to some extent of one or more of the symptoms associated with the specific disease or condition; reduced morbidity and mortality, and improvement in quality of life issues. The above parameters for assessing successful treatment and improvement in the disease are readily measurable by routine procedures familiar to a physician.

“Vaccine” refers to a compound that, once administered to a patient, may induce a humoral and/or cellular immune response, and this immune response is protective.

“Vector”, or “expression vector” means the vehicle by which a DNA or RNA sequence (e.g. a foreign gene) can be introduced into a host cell, so as to transform the host and promote expression (e.g. transcription and translation) of the introduced sequence.

### DETAILED DESCRIPTION

The present invention relates to a method for identifying Human endogenous retroviruses (HERVs)-derived T cells epitopes associated with a cancer, preferably associated with at least one cancer.

In one embodiment, all the steps of the method are performed in silico, and the method of the invention is an in silico method.

The method of the present invention notably allows to select a limited number of HERV-derived T cell epitopes specifically overexpressed by tumor cells and most likely to be immunogenic among a large number of HERV candidates.

In one embodiment, the method enables to select shared epitopes, i.e. epitopes shared in several patients with the same cancer, and/or epitopes shared among different cancer types and/or epitopes shared among cancer-associated HERVs. In one embodiment, the epitope(s) identified by the method of the present invention is/are shared by several patients with the same cancer. In one embodiment, the epitope(s) identified by the method of the present invention is/are shared among different cancer types. In one embodiment, the epitope(s) identified by the method of the present invention is/are shared among cancer-associated HERVs.

In one embodiment, said method comprises at least 2, 3, 4, 5 or more steps described hereinbelow.

In one embodiment, the method described hereinabove comprises the step of identifying HERVs in tumor and normal samples. Said HERVs may be identified with HERV database.

Methods for identifying HERVs in samples are well known by the skilled artisan, and include, without limitation, the use of reference tools such as Hervquant (C. C. Smith, et al., Endogenous retroviral signatures predict immunotherapy response in clear cell renal cell carcinoma. Journal of Clinical Investigation. 128, 4804-4820 (2018)) or Telescope (Bendall et al., Telescope: Characterization of the retrotranscriptome by accurate estimation of transposable element expression, PLOS Comput Biol. 2019 Sep. 30; 15(9):e1006453).

In addition, HERVs database are described in the literature, see for example, L. Vargiu et al., Classification and characterization of human endogenous retroviruses; mosaic forms are common. Retrovirology. 13, 7 (2016). HERVs database may also be retrieved from Genbank database.

In one embodiment, a normal sample refers to a sample obtained from a subject not affected with a cancer or to a sample obtained in a peri-tumorous location from a subject affected with cancer.

In one embodiment, a tumor sample refers to a sample from a tumor tissues in a subject affected with a cancer.

In one embodiment, the method described hereinabove comprises the step of selecting HERVs associated with cancer, preferably associated with at least one cancer. This step notably enables the selection of the HERVs that are differentially expressed in tumor and in normal samples.

In one embodiment, the selection of HERVs associated with cancer comprises the step of comparing HERVs expression in tumor and in normal samples. Said comparison may be done at the RNA level, meaning that the expression of RNA sequences is compared for each HERV between tumor and normal samples.

Said RNA sequences may be obtained from RNA sequencing database and/or by extraction from fresh tissues.

In one embodiment, a HERV is associated with a cancer, preferably associated with at least one cancer, if said HERV is expressed more than 2, 2.5, 3, 3.5, 4, 4.5, 5-fold or more in tumor samples than in normal samples. In one embodiment, a HERV is associated with a cancer, preferably associated with at least one cancer, if said HERV is expressed more than 2-fold in tumor samples than in normal samples and not more than 2-fold in any normal tissue compared to its matched tumor.

In one embodiment, the method described hereinabove comprises the step of selecting HERVs associated with a cytotoxic T cells response. This step notably enables the selection of HERVs that can induce an immune response.

In one embodiment, the association of the HERVs with a cytotoxic T cells response is assessed by a phenotype criterium. In one embodiment, the association of the HERVs with a cytotoxic T cells response is assessed by the association of each HERV with at least one CD4 or CD8 T cell signature.

In one embodiment, the association of the HERVs with a cytotoxic T cells response is assessed by determining the association of each HERV with a transcriptomic signature associated with at least one of CD4 or CD8 T cell phenotype. In one embodiment, this step enables to select HERVs associated with transcripts suggestive of the presence of cells with a CD4 or CD8 T cell phenotype in the tumor sample.

Examples of methods to assign CD4 or CD8 T cell phenotypes are well known by the skilled artisan in the art and include, for example, Xcell method (D. Aran et al., xCell: digitally portraying the tissue cellular heterogeneity landscape. Genome Biol. 18, 220 (2017)), and MCP counter.

As used herein, Xcell is a gene signature-based method that performs cell type enrichment analysis from gene expression data for 64 immune and stroma cell types. xCell signatures were validated using extensive in-silico simulations and also cytometry immunophenotyping. The xCell R package for generating the cell type scores and R scripts for the development of xCell are available at https://github.com/dviraran/xCell and deposited to Zenodo (assigned DOI http://doi.org/10.5281/zenodo.1004662).

In one embodiment, the signature to assign CD4 or CD8 T cell phenotype is based on the signatures of Xcell.

In one embodiment, the signature to assign CD4 T cell phenotype, in particular CD4 memory T cell phenotype, comprises or consists of the following genes: CD6, CD28, GPR183, EIF3E, ITK, PKP4, PREPL, DNAJA2, PTGES3, CD2AP and ICOS.

In one embodiment, the signature to assign CD4 T cell phenotype, in particular CD4 naive T cell phenotype, comprises or consists of the following genes: CD27, CLC, CTSW, DNAJB1, RBL2, HAUS3, ANKRD55, ZNF394 and CHMP7.

In one embodiment, the signature to assign CD4 T cell phenotype comprises or consists of the following genes: APBB1, CD28, CTLA4, ITK, PLCL1, SNPH, PPWD1, PHF3, ICOS, RAPGEF6, NOL9 and CHMP7.

In one embodiment, the signature to assign CD4 T cell phenotype, in particular CD4 central memory T cell phenotype (CD4 Tcm), comprises or consists of the following genes: CCR4, CCR8, CTLA4, DABI, ERNI, GPR15, DNAJB1, KRT1, POU6F1, TPO, TRADD, DLEC1, ICOS, TRAT1, FXYD7, FBXL8, SIRPG, ANKRD55 and OBSCN.

In one embodiment, the signature to assign CD4 T cell phenotype, in particular CD4 effector memory T cell phenotype (CD4 Tem), comprises or consists of the following genes: RPN2, SLAMF1, SPTAN1, TRADD, MYO16, MCF2L2, ESYT1, TRAPPC2L, ARHGAP15 and RIC8A.

In one embodiment, the signature to assign CD8 T cell phenotype, in particular CD8 naive T cell phenotype, comprises or consists of the following genes: CD8A, CD8B, EEF1D, GPR15, MYL1, NDUFA4, NDUFS5, PSG11, SKI, SON, HISTIH3A, BUD31, GPR52, ZNHIT3, CGRRF1, RRP8, NGDN, C19orf53, SETD2, MED31, SS18L2, CDK5RAP1, DDX24, PCIF1, MS4A5, HAUS3, LIN28A and TNKS2.

In one embodiment, the signature to assign CD8 T cell phenotype comprises or consists of the following genes: CASP8, CD8A, CD8B, GZMK, PTGDR, SLC1A7, TSPAN32, KLRG1, NPRL2, GIMAP4, CRTAM and ZNF611.

In one embodiment, the signature to assign CD8 T cell phenotype, in particular CD8 central memory T cell phenotype (CD8 Tcm), comprises or consists of the following genes: CASP8, CD27, TNFSF8, GZMK, NCKI, GPR171, CRTAM, PARP11 and TMEM30B.

In one embodiment, the signature to assign CD8 T cell phenotype, in particular CD8 effector memory T cell phenotype (CD8 Tem), comprises or consists of the following genes: DHX8, GZMH, GZMK, LAG3, ZAP70, COLQ, RGS9, CXCR6, PVRIG and PYH1N1.

In one embodiment, the signature to assign CD4 or CD8 T cell phenotype is based on the signature of MCP counter.

In one embodiment, the signature to assign CD4 or CD8 T cell phenotype comprises or consists of the following genes: CD28, CD3D, CD3G, CD5, CD6, CHRM3-AS2, CTLA4, FLT3LG, ICOS, MAL, MGC40069, PBX4, SIRPG, THEMIS, TNFRSF25, TRAT1, CD8B, CD8A, EOMES, FGFBP2, GNLY, KLRC3, KLRC4, KLRD1, BANK1, CD19, CD22, CD79A, CR2, FCRL2, IGKC, MS4A1, PAX5, CD160, KIR2DL1, KIR2DL3, KIR2DL4, KIR3DL1, KIR3DS1, NCRI, PTGDR, SH2DIB, ADAP2, CSFIR, FPR3, KYNU, PLA2G7, RASSF4, TFEC, CDIA, CDIB, CDIE, CLEC10A, CLIC2, WFDC21P, CA4, CEACAM3, CXCR1, CXCR2, CYP4F3, FCGR3B, HAL, KCNJ15, MEGF9, SLC25A37, STEAP4, TECPR2, TLE3, TNFRSF10C, VNN3, ACVRL1, APLN, BCL6B, BMP6, BMX, CDH5, CLEC14A, CXorf36, EDN1, ELTD1, EMCN, ESAM, ESM1, FAM124B, HECW2, HHIP, KDR, MMRN1, MMRN2, MYCT1, PALMD, PEAR1, PGF, PLXNA2, PTPRB, ROBO4, SDPR, SHANK3, SHE, TEK, TIE1, VEPH1, VWF, COL1A1, COL3A1, COL6A1, COL6A2, DCN, GREM1, PAMR1 and TAGLN.

In one embodiment, the association of the HERVs with a cytotoxic T cells response is assessed by determining the association of each HERV with a function signature. In one embodiment, said function signature is interferon (IFN)-γ signature or cytolytic activity.

In one embodiment, the IFN-γ signature is based on genes up-regulated in response to IFN-γ. In one embodiment, the association of the HERVs with a cytotoxic T cells response is assessed by determining the association of each HERV with a transcriptomic signature associated with an IFN-γ response.

In one embodiment, IFN-γ signature is determined on the basis of database. As an example, IFN-γ signature database may be accessible through Molecular Signature Database.

In one embodiment, the IFN-γ signature comprises at least 1, 2, 3, 4, 5, 6, 7, 8, 9, 10, 20, 40, 60, 80, 100, 120, 140, 160, 180 or 200 or all of the following genes: ADAR, APOL6, ARID5B, ARLAA, AUTS2, B2M, BANK1, BATF2, BPGM, BST2, BTG1, CIR, C1S, CASP1, CASP3, CASP4, CASP7, CASP8, CCL2, CCL5, CCL7, CD274, CD38, CD40, CD69, CD74, CD86, CDKN1A, CFB, CFH, CIITA, CMKLRI, CMPK2, CSF2RB, CXCL10, CXCL11, CXCL9, DDX58, DDX60, DHX58, EIF2AK2, EIF4E3, EPSTI1, FAS, FCGR1A, FGL2, FPR1, FTSJD2, GBP4, GBP6, GCH1, GPR18, GZMA, HERC6, HIF1A, HLA-A, HLA-B, HLA-DMA, HLA-DQA1, HLA-DRB1, HLA-G, ICAM1, IDO1, IFI27, IFI30, IFI35, IFI44, IFI44L, IFIH1, IFIT1, IFIT2, IFIT3, IFITM2, IFITM3, IFNAR2, IL10RA, IL15, IL15RA, IL18BP, IL2RB, ILAR, IL6, IL7, IRF1, IRF2, IRF4, IRF5, IRF7, IRF8, IRF9, ISG15, ISG20, ISOC1, ITGB7, JAK2, KLRK1, LAP3, LATS2, LCP2, LGALS3BP, LY6E, LYSMD2, 1-Mar, METTL7B, MT2A, MTHFD2, MVP, MX1, MX2, MYD88, NAMPT, NCOA3, NFKB1, NFKBIA, NLRC5, NM1, NOD1, NUP93, OAS2, OAS3, OASL, OGFR, P2RY14, PARP12, PARP14, PDE4B, PELI1, PFKP, PIM1, PLA2G4A, PLSCR1, PML, PNP, PNPT1, PRIC285, PSMA2, PSMA3, PSMB10, PSMB2, PSMB8, PSMB9, PSME1, PSME2, PTGS2, PTPN1, PTPN2, PTPN6, RAPGEF6, RBCK1, RIPK1, RIPK2, RNF213, RNF31, RSAD2, RTP4, SAMD9L, SAMHD1, SECTM1, SELP, SERPING1, SLAMF7, SLC25A28, SOCS1, SOCS3, SOD2, SP110, SPPL2A, SR1, SSPN, ST3GAL5, ST8SIA4, STAT1, STAT2, STAT3, STAT4, TAP1, TAPBP, TDRD7, TNFA1P2, TNFA1P3, TNFA1P6, TNFSF10, TOR1B, TRAFD1, TRIM14, TRIM21, TRIM25, TRIM26, TXNIP, UBE2L6, UPP1, USP18, VAMP5, VAMP8, VCAM1, WARS, XAF1, XCL1, ZBP1 and ZNFX1.

In one embodiment, the IFN-γ signature comprises all the genes mentioned hereinabove. In one embodiment, the IFN-γ signature is HALLMARK_INTERFERON_GAMMA_RESPONSE (https://www.gsea msigdb.org/gsea/msigdb/cards/HALLMARK_INTERFERON_GAMMA_RESPONSE. html).

In one embodiment, the IFN-γ signature is evaluated by calculating the enrichments scores based on IFN-γ signature database for each sample per cancer type.

In one embodiment, the cytolytic activity is evaluated by assessing the expression levels of granzyme-A (GZMA) and perforin (PRF1). In one embodiment, the cytolytic activity is evaluated by the calculation of the geometric mean of the granzyme-A (GZMA) and perforin (PRF1) level expression.

In one embodiment, the expression level of granzyme-A (GZMA) and perforin (PRF1) is evaluated at the transcriptomic level. In one embodiment, the expression level of granzyme-A (GZMA) and perforin (PRF1) is evaluated by RNA-seq.

As used herein, RNA-Seq (named as an abbreviation of RNA sequencing) is a sequencing technique which uses next-generation sequencing (NGS) to reveal the presence and quantity of specific RNA in a biological sample at a given moment.

RNA-seq data may be found in database, such as the ones of NCBI Gene Expression Omnibus (GEO) portal.

In one embodiment, the expression level of granzyme-A (GZMA) and perforin (PRF1) is evaluated at the proteomic level.

In one embodiment, the association of the HERVs with a cytotoxic T cells response is assessed by the absence of expression of said HERVs in normal purified T or NK cells. This step further enables to select HERVs expressed in tumoral cells, and not in T or NK cells.

Examples of HERVs expressed in T or NK cells are known by the skilled artisan in the art, and include, without limitation, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40 and SEQ ID NO: 41.

In one embodiment, the association of the HERVs with a cytotoxic T cells response is assessed by the evaluation of one, two or the three parameter(s) described hereinabove.

In one embodiment, the association of the HERVs with a cytotoxic T cells response is assessed by (i) the association of each HERV with at least one CD4 or CD8 T cell signature, (ii) the association of each HERV with a function signature being either IFN-γ signature or cytolytic activity, and/or (iii) the absence of expression of each HERV in normal purified T or NK cells.

In one embodiment, the association of the HERVs with one, two, or the three parameter(s) described hereinabove is assessed by a machine learning-based approach. In one embodiment, a machine learning-based approach is used to test the associations independently for each cancer type. As used herein, machine-learning refers to artificial intelligence, wherein algorithms build models based on training data.

In one embodiment, the association of the HERVs with one of the parameters described hereinabove is evaluated by a regression, preferably a L1 penalized regression (LASSO). In particular, a model may be built for each cancer type, wherein each HERV with a positive coefficient in the model is considered to be associated with the parameter.

In one embodiment, the method described hereinabove comprises a step of selecting T cell epitopes, in particular selecting shared T cell epitopes.

This step notably enables the selection of epitopes i) having common regions with known HERV proteins, reducing thereby the risk of selecting non-translated sequences, and ii) being strong binders for the MHC class I molecules, allowing their presentation by MHC class I molecules. This step may further enable the selection of epitopes iii) being among the most shared epitopes in HERVs, such as the HERVs associated with cancer.

In one embodiment, the method comprises a step of selecting T cell epitope. In one embodiment, the step of selecting T cell epitopes comprises a step of determining putative epitopes.

In one embodiment, the step of selecting T cell epitopes comprises a step of translating HERV sequences into 1, 2, 3, 4, 5 or 6 possible frames and identifying open-reading frames (ORFs) of at least 10, 11, 12, 13, 14, 15, or more amino acids to obtain putative epitopes.

In one embodiment, the step of selecting T cell epitopes comprises a step of predicting the binding of the putative epitopes with MHC class I molecules.

Analysis tools for predicting the binding of sequences with MHC molecules are well known by the skilled artisan in the art and include, for example, MHCflurry (T. J. O'Donnell et al., MHCflurry: Open-Source Class I MHC Binding Affinity Prediction. Cell Systems. 7, 129-132.e4 (2018)) or NetMCH I & II.

In one embodiment, said MCH class I molecule is an HLA molecule. In one embodiment, the MCH class I molecule is selected from the group comprising or consisting of HLA-A, HLA-B and HLA-C molecules. In one embodiment, the MCH class I molecule is an HLA-A molecule, such as an HLA-A2 molecule.

In one embodiment, only T cell epitopes predicted as capable of binding with MHC class I molecules are selected. In one embodiment, the epitope is selected if its sequence is predicted as a strong binder of a MCH class I molecule as defined hereinabove.

In one embodiment, the step of selecting T cell epitopes comprises a step of aligning the epitopes with sequences of known HERV proteins, to reduce the risk of selecting non-translated sequences.

In one embodiment, the HERV protein is an envelop (Env) protein. In one embodiment, the HERV protein is a group-specific antigen (Gag) protein. In one embodiment, the HERV protein is a polymerase (Pol). In one embodiment, the HERV protein is a protease (Pro). In one embodiment, the HERV protein is a Rec protein. In one embodiment, the HERV protein is an accessory protein.

In one embodiment, the HERV protein is an HERV-K/HML-2 protein. In one embodiment, the HERV protein is a HERV-K/HML-2 Gag protein. In one embodiment, the HERV protein is a HERV-K/HML-2 Pol protein.

Examples of HERV proteins include, without limitation, HERV-K10 Gag protein (SEQ ID NO: 20), HERV-K113 Gag protein (SEQ ID NO: 21), HERV-K21 Gag protein (SEQ ID NO: 22), HERV-K113 Pol protein (SEQ ID NO: 23), HERV-K9 Pol protein (SEQ ID NO: 24), HERV-K6 Pol protein (SEQ ID NO: 25), HERV-K113 Env protein (SEQ ID NO: 26).

SEQ ID NO: 20
MGQTKSKIKSKYASYLSFIKILLKRGGVKVSTKNLIKLFQIIEQF
CPWFPEQGTSDLKDWKRIGKELKQAGRKGNIIPLTVWNDWAIIKA
ALEPFQTEEDSISVSDAPGSCLIDCNENTRKKSQKETESLHCEYV
AEPVMAQSTQNVDYNQLQEVIYPETLKLEGKGPELMGPSESKPRG
TSPLPAGQVLVRLQPQKQVKENKTQPQVAYQYWPLAELQYRPPPE
SQYGYPGMPPAPQGRAPYHQPPTRRLNPMAPPSRQGSELHEIIDK
SRKEGDTEAWQFPVTLEPMPPGEGAQEGEPPTVEARYKSFSIKML
KDMKEGVKQYGPNSPYMRTLLDSIAYGHRLIPYDWEILAKSSLSP
SQFLQFKTWWIDGVQEQVRRNRAANPPVNIDADQLLGIGQNWSTI
SQQALMQNEAIEQVRAICLRAWEKIQDPGSTCPSFNTVRQGSKEP
YPDFVARLQDVAQKSIADEKAGKVIVELMAYENANPECQSAIKPL
KGKVPAGSDVISEYVKACDGIGGAMHKAMLMAQAITGVVLGGQVR
TFGGKCYNCGQIGHLKKNCPVLNKQNITIQATTTGREPPDLCPRC
KKGKHWASQCRSKFDKNGQPLSGNEQRGQPQAPQQTGAFPIQPFV
PQGFQGQQPPLSQVFQGISQLPQYNNCPSPQAAVQQ
SEQ ID NO: 21
MGQTKSKIKSKYASYLSFIKILLKRGGVKVSTKNLIKLFQIIEQF
CPWFPEQGTLDLKDWKRIGKELKQAGRKGNIIPLTVWNDWAIIKA
ALEPFQTEEDSVSVSDAPGSCIIDCNEKTRKKSQKETESLHCEYV
AEPVMAQSTQNADYNQLQEVIYPETLKLEGKGPELMGPSESKPRG
TSPLPAGQVPVTLQPQKQVKENKTQPPVAYQYWPPAELQYQPPPE
SQYGYPGMPPAPQGRAPYPQPPTRRLNPTAPPSRQGSELHEIIDK
SRKEGDTEAWQFPVTLELMPPGEGAQEGEPPTVEARYKSFSIKML
KDMKEGVKQYGPNSPYMRTLLDSIAHGHRLIPYDWEILAKSSLSP
SQFLQFKTWWIDGVQEQVRRNRAANPPVNIDADQLLGIGQNWSTI
SQQALMQNEAIEQVRAICLRAWEKIQDPGSTCPSFNTVRQGSKEP
YPDFVARLQDVAQKSIADEKARKVIVELMAYENANPECQSAIKPL
KGKVPAGSDVISEYVKACDGMGGAMHKAMLMAQAITGVVLGGQVR
TFGGKCYNCGQIGHLKKNCPVLNKQNITIQATTTGREPPDLCPRC
KKGKHWASQCRSKFDKNGQPLSGNEQRGQPQAPQQTGAFPIQPFV
PQGFQGQQPPLSQVFQGISQLPQYNNCPPPQAAVQQ
SEQ ID NO: 22
MGQTKSKIKSKYASYLSFIKILLKRGGVKVSTKNLIKLFQIIEQF
CPWFPEQGTLDLKDWKRIGKELKQAGRKGNIIPLTVWNDWAIIKA
ALEPFQTEEDSISVSDAPGSCIIDCNENTRKKSQKETEGLHCEYA
AEPVMAQSTQNVDYNQLQEVIYPETLKLEGKGPELVGPSESKPRG
TSPLPAGQVPVTLQPQTQVKENKTQPPVAYQYWPPAELQYRPPPE
SQYGYPGMPPAPQGRAPYPQPPTRRLNPTAPPSRQGSELHEIIDK
SKEGDTEAWQFPVMLEPMPPGEGAQEGEPPTVEARYKSFSIKMLK
DMKEGVKQYGPNSPYMRTLLDSIAHGHRLIPYDWEILAKSSLLPS
QFLQFKTWWIDGVQEQVQRNRAANPPVNIDADQLLGIGQNWSTIS
QQALMQNEAIEQVRAICLRAWEKIQDPGSTCPSFNTVRQSSKEPY
PDFVARLQDVAQKSIADEKARKVIVELMAYENANPECQSAIKPLK
GKVPAGSDVISEYVKACDGIGGAMHKAMLMAQAITGVVLGGQVRT
FGGKCYNCGQIGHLKKNCPVLNKQNITIQATTTGREPPDLCPRCK
KGKHWASQCRSKFDKNGQPLSGNEQRGQPQAPQQTGAFPIQPFVP
QGFQGQQPPLSQVFQGISQLPQYNNCPPPQAAVQQ
SEQ ID NO: 23
NKSRKRRNRVSFLGAATVEPPKPIPLTWKTEKPVWVNQWPLPKQK
LEALHLLANEQLEKGHIEPSFSPWNSPVFVIQKKSGKWRMLTDLR
AVNAVIQPMGPLQPGLPSPAMIPKDWPLIIIDLKDCFFTIPLAEQ
DCEKFAFTIPAINNKEPATRFQWKVLPQGMLNSPTICQTFVGRAL
QPVRDKFSDCYIIHYIDDILCAAETKDKLIDCYTFLQAEVANAGL
AIASDKIQTSTPFHYLGMQIENRKIKPQKIEIRKDTLKTLNDFQK
LLGDINWIRPTLGIPTYVMSNLFSILRGDSDLNSKRMLTPETTKE
IKLVEEKIQSAQINRIDPLAPLRLLIFATAHSPIGIIIQNTDLVE
WSFLPHSTVKTFTLYLDQIATLIGQTRLRIIKLCGNDPDKIVVPL
TKEQVRQAFINSGAWQIGLANFVGIIDNHYPKTKIFQFLKLTTWI
LPKITRREPLENALTVFTDGSSNGKAAYTGLKERVIKTPYQSAQR
AELVAVITVLQDFDQPINIISDSAYVVQATRDVETALIKYSMDDQ
LNQLFNLLQQTVRKRNFPFYITHIRAHTNLPGPLTKANEQADLLV
SSALIKAQELHALTHVNAAGLKNKFDVTWKQAKDIVQHCTQCQVL
HLPTQEAGVNPRGLCPNALWQMDVTHVPSFGRLSYVHVTVDTYSH
FIWATCQTGESTSHVKKHLLSCFAVMGVPEKIKTDNGPGYCSKAF
QKFLSQWKISHTTGIPYNSQGQAIVERTNRTLKTQLVKQKEGGDS
KECTTPQMQLNLAPYTLNFLNIYRNQTTTSAEQHLTGKKNSPHEG
KLIWWKDNKNKTWEIGKVITWGRGFACVSPGENQLPVWMPTRHLK
FYNEPIGDAKKSTSAETETPQSSTVDSQDEQNGDVRRTDEVAIHQ
EGRAADLGTTKEADAVSYKISREHKGDTNPREYAACSLDDCINGG
KSPYACRSSCS
SEQ ID NO: 24
MGQTKSKIKSKYASYLSFIKILLKRGGVKVSTKNLIKLFQIIEQF
CPWFPEQGTLDLKDWKRIGKELKQAGRKGNIIPLTVWNDWAIIKA
ALEPFQTEEDSISVSDAPGSGIIDCNEKTRKKSQKETESLHCEYV
AEPVMAQSTQNVDYNQLQEVIYPETLKLEGKGPELVGPSESKPRG
TSPLPAGQVPVTLQPQKQVKENKTQPPVAYQYWPPAELQYRPPPE
SQYGYPGMPPAPQGRAPYPQPPTRRLNPTAPPSRQGSELHEIIDK
SRKEGDTEAWQFPVTLEPMPPGEGAQEGEPPTVEARYKSFSIKIL
KDMKEGVKQYGPNSPYMRTLLDSIAHGHRLIPYDWEILAKSSLSP
SQFLQFKTWWIDGVQEQVRRNRAANPPVNIDADQLLGIGQNWSTI
SQQALMQNEAIEQVRAICLRAWEKIQDPGSTCPSFNTVRQGSKEP
YPDFVARLQDVAQKSIADEKARKVIVELMAYENANPECQSAIKPL
KGKVPAGSDVISEYVKACDGIGGAMHKAMLMAQAITGVVLGGQVR
TFGGKCYNCGQIGHLKKNCPVLNKQNITIQATTTGREPPDLCPRC
KKGKHWASQCRSKFDKNGQPLSGNEQRGQPQAPQQTGAFPIQPFV
PQGFQGQQPPLSQVFQGISQLPQYNNCPPPQVAVQQVDLCTIQAV
SLLPGEPPQKIPTGVYGPLPEGTVGLILGRSSLNLKGVQIHTSVV
DSDYKGEIQLVISSSVPWSASPGDRIAQLLLLPYIKGGNSEIKRI
GGLGSTDPTGKAAYWASQVSENRPVCKAIIQGKQFEGLVDTGADV
SIIALNQWPKNWPKQKAVTGLVGIGTASEVYQSMEILHCLGPDNQ
ESTVQPMITSIPLNLWGRDLLQQWGAEITMPAPLYSPTSQKIMTK
RGYIPGKGLGKNEDGIKIPFEAKINQKREGIGYPFLGAATIEPPK
PIPLTWKTEKPVWVNQWPLPKQKLEALHLLANEQLEKGHIEPSFS
PWNSPVFVIQKKSGKWRMLTDLRAVNAVIQPMGPLQPGLPSPAMI
PKDWPLIIIDLKDCFFTIPLAEQDCEKFAFTIPAINNKEPATRFQ
WKVLPQGMLNSPTICQTFVGRALQPVKVFRLLYYSLY
SEQ ID NO: 25
NKSRKRRNRESLLGAATVEPPKPIPLTWKTEKPVWVNQWPLPKQK
LEALHLLANEQLEKGHIEPSFSPWNSPVFVIQKKSGKWRMLTDLR
AVNAVIQPMGPLQPGLPSPAMIPKDWPLIIIDLKDCFFTIPLAEQ
DCEKFAFTIPAINNKEPATRFQWKVLPQGMLNSPTICQTFVGRAL
QPVREKFSDCYIIHCIDDILCAAETKDKLIDCYTFLQAEVANAGL
AIASDKIQTSTPFHYLGMQIENRKIKPQKIEIRKDTLKTLNDFQK
LLGDINWIRPTLGIPTYAMSNLFSILRGDSDLNSKRMLTPEATKE
IKLVEEKIQSAQINRIDPLAPLQLLIFATAHSPTGIIIQNTDLVE
WSFLPHSTVKTFTLYLDQIATLIGQTRLRIIKLCGNDPDKIVVPL
TKEQVRQAFINSGAWKIGLANFVGIIDNHYPKTKIFQFLKLTTWI
LPKITRREPLENALTVFTDGSSNGKAAYTGPKERVIKTPYQSAQR
AELVAVITVLQDFDQPINIISDSAYVVQATRDVETALIKYSMDDQ
LNQLENLLQQTVRKRNFPFYITHIRAHTNLPGPLTKANEQADLLV
SSALIKAQELHALTHVNAAGLKNKFDVTWKQAKDIVQHCTQCQVL
HLPTQEAGVNPRGLCPNALWQMDVTHVPSFGRLSYVHVTVDTYSH
FIWATCQTGESTSHVKKHLLSCFAVMGVPEKIKTDNGPGYCSKAF
QKFLSQWKISHTTGIPYNSQGQAIVERTNRTLKTQLVKQKEGGDS
KECTTPQMQLNLALYTLNFLNIYRNQTTTSAEQHLTGKKNSPHEG
KLIWWKDNKNKTWEIGKVITWGRGFACVSPGENQLPVWIPTRHLK
FYNEPIRDAKKSTSAETETSQSSTVDSQDEQNGDVRRTDEVAIHQ
EGRAANLGTTKEADAVSYKISREHKGDTNPREYAACSLDDCINGG
KSPYACRSSCS
SEQ ID NO: 26
MNPSEMQRKAPPRRRRHRNRAPLTHKMNKMVTSEEQMKLPSTKKA
EPPTWAQLKKLTQLATKYLENTKVTQTPESMLLAALMIVSMVVSL
PMPAGAAAANYTYWAYVPFPPLIRAVTWMDNPIEIYVNDSVWVPG
PTDDCCPAKPEEEGMMINISIGYRYPPICLGRAPGCLMPAVQNWL
VEVPTVSPISRFTYHMVSGMSLRPRVNYLQDFSYQRSLKFRPKGK
PCPKEIPKESKNTEVLVWEECVANSAVILQNNEFGTLIDWAPRGQ
FYHNCSGQTQSCPSAQVSPAVDSDLTESLDKHKHKKLQSFYPWEW
GEKGISTARPKIISPVSGPEHPELWRLTVASHHIRIWSGNQTLET
RDRKPFYTIDLNSSLTVPLQSCVKPPYMLVVGNIVIKPDSQTITC
ENCRLLTCIDSTFNWQHRILLVRAREGVWIPVSMDRPWEASPSVH
ILTEVLKGVLNRSKRFIFTLIAVIMGLIAVTATAAVAGVALHSSV
QSVNFVNDWQNNSTRLWNSQSSIDQKLANQINDLRQTVIWMGDRL
MSLEHRFQLQCDWNTSDFCITPQIYNESEHHWDMVRCHLQGREDN
LTLDISKLKEQIFEASKAHLNLVPGTEAIAGVADGLANLNTVTWV
KTIGSTTIINLILILVCLFCLLLVYRCTQQLRRDSDHRERAMMTM
VVLSKRKGGNVGKSKRDQIVTVSV

In one embodiment, the epitope is selected if its sequence shares at least 70, 75, 80, 85, 90, 95, 96, 97, 98, 99% or more identity with at least one HERV protein as defined hereinabove. In one embodiment, the sequence is selected if said sequence shares at least 90% of identity with at least one HERV protein as defined hereinabove.

In one embodiment, an epitope is selected if its sequence shares at least 70, 75, 80, 85, 90, 95, 96, 97, 98, 99% or more identity with a HERV protein as defined hereinabove and/or if said epitope is predicted to bind with MHC class I molecules as described hereinabove.

In one embodiment, the method described hereinabove comprises a step of selecting epitopes among the most shared epitopes in the HERVs identified in one of the steps described hereinabove. This step notably enables to select the epitopes that are among the most shared epitopes in the HERVs, such as the HERVs associated with cancer.

Said step may lead to the selection of 1, 2, 3, 4, 5, 6, 7, 8, 9, 10 or more epitopes among the most shared epitopes in the HERVs identified in one of the steps described previously, such as the HERVs associated with cancer.

In one embodiment, the method described hereinabove comprises the step of assessing the expression of the epitopes identified by one of the steps described hereinabove at the protein or peptide level in tumor samples. Said step notably enables to confirm that the selected epitopes are translated or expressed in tumor samples.

In one embodiment, said expression of epitopes at the protein or peptide level is evaluated in silico by using one or more proteomic database(s).

The proteomic database may be obtained from tandem mass spectrometry (MS/MS) data. As used herein, tandem mass spectrometry (TANDEM MS), also named MS/MS, refers to a mass spectrometry technique using two or more mass analyzers. With two in tandem, the precursor ions are mass-selected by a first mass analyzer, and focused into a collision region where they are then fragmented into product ions which are then characterized by a second mass analyzer.

Analysis tools for evaluating the expression of proteins or peptides from database are well known by the skill artisan in the art, and include, for example, pepquery (B. Wen et al., PepQuery enables fast, accurate, and convenient proteomic validation of novel genomic alterations. Genome Res. 29, 485-493 (2019)).

In one embodiment, said expression of epitopes at the protein or peptide level is evaluated in vitro by immunopeptidomics analysis. Immunopeptidomics analysis enables to assess the presence of the epitopes associated with HLA molecules from tumors.

In one embodiment, an epitope is selected if said epitope shows evidence of translation in tumor samples, and/or of translation and association with HLA molecules in tumor samples.

In one embodiment, the method as described hereinabove comprises the following steps:
(a). Identifying HERVs associated with a cancer, preferably associated with at least one cancer, and
(b). Selecting T cell epitopes among the HERVs identified in the previous step, and
   wherein said method further comprises at least one, preferably two, of the following steps:
(i). Selecting HERVs associated with a cytotoxic T cells response among the cancer-associated HERVs identified in step (a), said step being between step (a) and step (b), and/or
(ii). Assessing the expression at the protein or peptide level of the HERVs-derived T cells epitopes identified in step (b) in tumor samples, said step being after step (b).

In one embodiment, steps (a) and (b) and steps (i) and/or (ii) are in silico steps. In one embodiment, step (ii) is an in vitro step.

In one embodiment, step (a) comprises the step of identifying and selecting HERVs associated with a cancer, preferably associated with at least one cancer.

In one embodiment, step (b) comprises the step of selecting shared T cell epitopes.

In one embodiment, the method as described hereinabove comprises the following steps:
(a). Identifying HERVs associated with a cancer, preferably associated with at least one cancer, and
(b). Selecting T cell epitopes among the HERVs identified in the previous step, and
   wherein said method further comprises at least one, preferably two, of the following steps:
(i). Selecting HERVs associated with a cytotoxic T cells response among the cancer-associated HERVs identified in step (a), said step being between the step (a) and the step (b), and/or
(ii). Assessing the expression at the protein or peptide level of the HERVs-derived T cells epitopes identified in step (b) in tumor samples, said step being after the step (b), wherein the steps (a), (b), (i) and (ii) are performed in silico.

Thus, in one embodiment, the in silico method as described hereinabove comprises the following steps:
(a). Identifying HERVs associated with a cancer, preferably associated with at least one cancer, and
(b). Selecting T cell epitopes among the HERVs identified in the previous step, and
   wherein said method further comprises at least one, preferably two, of the following steps:
(i). Selecting HERVs associated with a cytotoxic T cells response among the cancer-associated HERVs identified in step (a), said step being between the step (a) and the step (b), and/or
(ii). Assessing the expression at the protein or peptide level of the HERVs-derived T cells epitopes identified in step (b) in tumor samples, said step being after the step (b).

In one embodiment, said method further comprises, after the step (b) or before the step (ii), a step of selecting epitopes among the most shared epitopes in the HERVs associated with cancer identified in step (a).

In one embodiment, the in silico method as described hereinabove comprises the following steps:
(a) Identifying HERVs associated with a cancer, preferably associated with at least one cancer,
(b) Selecting T cell epitopes among the cancer-associated HERVs identified in step (a), and
(c) Assessing the expression at the protein or peptide level of the HERVs-derived T cells epitopes identified in step (b) in tumor samples.

In one embodiment, the in silico method as described hereinabove comprises the following steps:
(a) Identifying HERVs associated with a cancer, preferably associated with at least one cancer,
(b) Selecting HERVs associated with a cytotoxic T cells response among the cancer-associated HERVs identified in step (a),
(c) Selecting T cell epitopes among the HERVs identified in step (b), and
(d) Assessing the expression at the protein or peptide level of the HERVs-derived T cells epitopes identified in step (c) in tumor samples.

In one embodiment, the in silico method as described hereinabove comprises the following steps:
(a) Identifying HERVs associated with a cancer, preferably associated with at least one cancer,
(b) Selecting T cell epitopes among the cancer-associated HERVs identified in step (a),
(c) Selecting epitopes among the most shared epitopes in the HERVs associated with cancer identified in step (a), and
(d) Assessing the expression at the protein or peptide level of the HERVs-derived T cells epitopes identified in step (c) in tumor samples.

In one embodiment, the in silico method described hereinabove comprises the following steps:
(a) Identifying HERVs associated with a cancer, preferably associated with at least one cancer,
(b) Selecting HERVs associated with a cytotoxic T cells response among the cancer-associated HERVs identified in step (a),
(c) Selecting T cell epitopes among the HERVs identified in step (b),
(d) Selecting epitopes among the most shared epitopes in the cancer-associated HERVs identified in step (a), and
(e) Assessing the expression at the protein or peptide level of the HERVs-derived T cells epitopes identified in step (d) in tumor samples.

In one embodiment, the in silico method as described hereinabove comprises the following steps:
(a) Identifying HERVs associated with a cancer, preferably associated with at least one cancer,
(b) Selecting HERVs associated with a cytotoxic T cells response among the cancer-associated HERVs identified in step (a), and
(c) Selecting T cell epitopes among the cancer-associated HERVs identified in step (b).

In one embodiment, the in silico method as described hereinabove comprises the following steps:
(a) Identifying HERVs associated with a cancer, preferably associated with at least one cancer,
(b) Selecting HERVs associated with a cytotoxic T cells response among the cancer-associated HERVs identified in step (a),
(c) Selecting T cell epitopes among the cancer-associated HERVs identified in step (b), and
(d) Selecting epitopes among the most shared epitopes in the cancer-associated HERVs identified in step (a).

In one embodiment, the steps are as described hereinabove.

In one embodiment, the in silico method described hereinabove comprises the following steps:
(a) Identifying HERVs associated with a cancer, preferably associated with at least one cancer,
(b) Selecting HERVs associated with a cytotoxic T cells response among the cancer-associated HERVs identified in step (a), wherein the association of the HERVs with a cytotoxic T cells response is assessed by (i) the association of each HERV with at least one CD4 or CD8 T cell signature, (ii) the association of each HERV with a function signature being either interferon (IFN)-γ signature or cytolytic activity, and/or (iii) the absence of expression of each HERV in normal purified T or NK cells.
(c) Selecting T cell epitopes among the HERVs identified in step (b),
   wherein the selection of the T cell epitopes comprises the steps of:
determining putative epitopes,
predicting the binding of the putative epitopes with MHC class I molecules,
optionally, aligning the epitopes with sequences of known HERV proteins,
(d) Optionally, selecting epitopes among the most shared epitopes in the cancer-associated HERVs, and
(e) Optionally, assessing the expression at the protein or peptide level of the HERVs-derived T cells epitopes identified in step (c) or (d) in tumor samples.

In one embodiment, the method as described hereinabove comprises the following steps:
(a). Identifying HERVs associated with a cancer, preferably associated with at least one cancer, and
(b). Selecting T cell epitopes among the HERVs identified in the previous step, and
   wherein said method further comprises at least one, preferably two, of the following steps:
(i). Selecting HERVs associated with a cytotoxic T cells response among the cancer-associated HERVs identified in step (a), said step being between the step (a) and the step (b), and/or
(ii). Assessing the expression at the protein or peptide level of the HERVs-derived T cells epitopes identified in step (b) in tumor samples, said step being after the step (b),
   wherein the steps (a), (b) and (i) are performed in silico, and the step (ii) is performed in vitro.

In one embodiment, step (a) comprises the step of identifying and selecting HERVs associated with a cancer, preferably associated with at least one cancer.

In one embodiment, step (b) comprises the step of selecting shared T cell epitopes.

In one embodiment, said method further comprises, after the step (b) or before the step (ii), a step of selecting epitopes among the most shared epitopes in the HERVs associated with cancer identified in step (a).

In one embodiment, the method as described hereinabove comprises the following steps:
(a) Identifying HERVs associated with a cancer, preferably associated with at least one cancer,
(b) Selecting T cell epitopes among the cancer-associated HERVs identified in step (a), and
(c) Assessing the expression at the protein or peptide level of the HERVs-derived T cells epitopes identified in step (b) in tumor samples,
   wherein the steps (a), (b) are performed in silico and the step (c) is performed in vitro.

In one embodiment, the method as described hereinabove comprises the following steps:
(c) Identifying HERVs associated with a cancer, preferably associated with at least one cancer,
(d) Selecting HERVs associated with a cytotoxic T cells response among the cancer-associated HERVs identified in step (a),
(c) Selecting T cell epitopes among the HERVs identified in step (b), and (d) Assessing the expression at the protein or peptide level of the HERVs-derived T cells epitopes identified in step (c) in tumor samples,
   wherein the steps (a), (b), (c) are performed in silico and the step (d) is performed in vitro.

In one embodiment, the method as described hereinabove comprises the following steps:
(a) Identifying HERVs associated with a cancer, preferably associated with at least one cancer,
(b) Selecting T cell epitopes among the cancer-associated HERVs identified in step (a),
(c) Selecting epitopes among the most shared epitopes in the HERVs associated with cancer identified in step (a), and
(d) Assessing the expression at the protein or peptide level of the HERVs-derived T cells epitopes identified in step (c) in tumor samples,
   wherein the steps (a), (b), (c) are performed in silico and the step (d) is performed in vitro.

In one embodiment, the method described hereinabove comprises the following steps:
(a) Identifying HERVs associated with a cancer, preferably associated with at least one cancer,
(b) Selecting HERVs associated with a cytotoxic T cells response among the cancer-associated HERVs identified in step (a),
(c) Selecting T cell epitopes among the HERVs identified in step (b),
(d) Selecting epitopes among the most shared epitopes in the cancer-associated HERVs identified in step (a), and
(e) Assessing the expression at the protein or peptide level of the HERVs-derived T cells epitopes identified in step (d) in tumor samples,
   wherein the steps (a), (b), (c), (d) are performed in silico and the step (e) is performed in vitro.

In one embodiment, the steps are as described hereinabove.

In one embodiment, the method described hereinabove comprises the following steps:
(a) Identifying HERVs associated with a cancer, preferably associated with at least one cancer,
(b) Selecting HERVs associated with a cytotoxic T cells response among the cancer-associated HERVs identified in step (a), wherein the association of the HERVs with a cytotoxic T cells response is assessed by (i) the association of each HERV with at least one CD4 or CD8 T cell signature, (ii) the association of each HERV with a function signature being either interferon (IFN)-γ signature or cytolytic activity, and/or (iii) the absence of expression of each HERV in normal purified T or NK cells.
(c) Selecting T cell epitopes among the HERVs identified in step (b),
   wherein the selection of the T cell epitopes comprises the steps of:
determining putative epitopes,
predicting the binding of the putative epitopes with MHC class I molecules,
optionally, aligning the epitopes with sequences of known HERV proteins,
(d) Optionally, selecting epitopes among the most shared epitopes in the cancer-associated HERVs, and
(e) Optionally, assessing the expression at the protein or peptide level of the HERVs-derived T cells epitopes identified in step (c) or (d) in tumor samples, wherein the steps (a), (b), (c), (d) are performed in silico and the step (e) is performed in vitro.

In one embodiment, the steps (d) and (e) are performed in parallel, when present. In one embodiment, the step of aligning the epitopes with sequences of known HERV proteins and the steps (d) and/or (e) are performed in parallel, when present.

In one embodiment, the methods described hereinabove further comprise the step of aligning the selected epitopes with human proteome. This step notably enables to confirm that the selected epitopes do not match any self-protein.

In one embodiment, the step of aligning the selected epitopes with human proteome is performed in parallel with the steps of aligning the epitopes with sequences of known HERV proteins, the step (d) and/or the step (e), when present.

In one embodiment, the methods described hereinabove are combined with an in vitro validation of the selected epitopes. In particular, the methods described hereinabove may be combined with one or several step(s) described hereinbelow.

In one embodiment, said in vitro validation comprises the step of evaluating the induction of T cell responses.

In one embodiment, the induction of T cells responses is assessed by measuring the induction of CD8+ T cells specific for the selected epitopes. Examples of methods to assess induction of CD8+ T cells include, for example, in vitro priming assays with the selected epitopes (see the Example part for example).

In one embodiment, the induction of T cells responses is assessed by measuring IFN-γ or granzyme B production in presence of epitopes-stimulated cells (see the Example part for example). Examples of methods for measuring IFN-γ or granzyme B production include, for example, flow cytometry or fluorospot assays.

In one embodiment, the induction of T cells responses is assessed by measuring degranulation markers, such as, for example, CD107a (see the Example part for example).

In one embodiment, said in vitro validation comprises a step of evaluating the affinity of the CD8+ T cells specific for the selected epitopes with MCH molecules. Examples of methods for evaluating such affinity include, without limitation, 3D modeling (see the Example part for example).

In one embodiment, said in vitro validation comprises a step of evaluating the functionality of the CD8+ T cells specific for the selected epitopes. Examples of methods to measure the functionality of CD8+ T cells include, without limitation, immune-cell killing assays and analyses of morphological signs of T-cells activation by microscopy (see the Example part for example).

In one embodiment, said in vitro validation comprises a step of assessing the presence of T cells specific for the selected epitopes in tumor tissues from a subject affected with cancer (see the Example part for example). Said tumor tissues may be obtained by biopsies.

The present invention also relates to a peptide comprising or consisting of an epitope identified by the method described hereinabove.

The present invention relates to a peptide comprising or consisting of an epitope having a sequence selected in the group comprising or consisting of KLLGDINWI (SEQ ID NO: 1), FIFTLIAVI (SEQ ID N: 2), RMLTDLRAV (SEQ ID NO: 3), FLSLYFVSV (SEQ ID NO: 4), TLIAVIMGL (SEQ ID NO: 5), YLDQIATLI (SEQ ID NO: 6), FLQAEVANA (SEQ ID NO: 7), WMGDRLMSL (SEQ ID NO: 8), ALHSSVQSV (SEQ ID NO: 9), ILTEVLKGV (SEQ ID NO: 10), LMAQAITGV (SEQ ID NO: 11), RLSYVHVTV (SEQ ID NO: 12), VLQDFDQPI (SEQ ID NO: 13), ALMIVSMVV (SEQ ID NO: 14), MLLAALMIV (SEQ ID NO: 15), YIDDILCAA (SEQ ID NO: 16), YIIHYIDDI (SEQ ID NO: 17), YIWCPTWRL (SEQ ID NO: 18), and YIWCPTWSL (SEQ ID NO: 19). The present invention relates to a peptide comprising or consisting of ID NO: 1), FIFTLIAVI (SEQ ID N: 2), RMLTDLRAV (SEQ ID NO: 3), FLSLYFVSV (SEQ ID NO: 4), TLIAVIMGL (SEQ ID NO: 5), YLDQIATLI (SEQ ID NO: 6), FLQAEVANA (SEQ ID NO: 7), WMGDRLMSL (SEQ ID NO: 8), ALHSSVQSV (SEQ ID NO: 9), ILTEVLKGV (SEQ ID NO: 10), LMAQAITGV (SEQ ID NO: 11), RLSYVHVTV (SEQ ID NO: 12), VLQDFDQPI (SEQ ID NO: 13), ALMIVSMVV (SEQ ID NO: 14), MLLAALMIV (SEQ ID NO: 15), YIDDILCAA (SEQ ID NO: 16), YIIHYIDDI (SEQ ID NO: 17), YIWCPTWRL (SEQ ID NO: 18), and YIWCPTWSL (SEQ ID NO: 19).

In one embodiment, the peptide comprises or consists of an epitope having a sequence selected in the group comprising or consisting of RMLTDLRAV (SEQ ID NO: 3), ALHSSVQSV (SEQ ID NO: 9), LMAQAITGV (SEQ ID NO: 11), VLQDFDQPI (SEQ ID NO: 13), ALMIVSMVV (SEQ ID NO: 14), MLLAALMIV (SEQ ID NO: 15), YIDDILCAA (SEQ ID NO: 16). In one embodiment, the peptide comprises or consists of a sequence selected in the group comprising or consisting of RMLTDLRAV (SEQ ID NO: 3), ALHSSVQSV (SEQ ID NO: 9), LMAQAITGV (SEQ ID NO: 11), VLQDFDQPI (SEQ ID NO: 13), ALMIVSMVV (SEQ ID NO: 14), MLLAALMIV (SEQ ID NO: 15), YIDDILCAA (SEQ ID NO: 16).

In one embodiment, the peptide comprises or consists of an epitope having a sequence selected in the group comprising or consisting of RMLTDLRAV (SEQ ID NO: 3), LMAQAITGV (SEQ ID NO: 11), VLQDFDQPI (SEQ ID NO: 13), ALMIVSMVV (SEQ ID NO: 14), MLLAALMIV (SEQ ID NO: 15), YIDDILCAA (SEQ ID NO: 16). In one embodiment, the peptide comprises or consists of a sequence selected in the group comprising or consisting of RMLTDLRAV (SEQ ID NO: 3), LMAQAITGV (SEQ ID NO: 11), VLQDFDQPI (SEQ ID NO: 13), ALMIVSMVV (SEQ ID NO: 14), MLLAALMIV (SEQ ID NO: 15), YIDDILCAA (SEQ ID NO: 16).

In one embodiment, the peptide comprises or consists of an epitope having a sequence selected in the group comprising or consisting of RMLTDLRAV (SEQ ID NO: 3), LMAQAITGV (SEQ ID NO: 11), VLQDFDQPI (SEQ ID NO: 13), ALMIVSMVV (SEQ ID NO: 14), YIDDILCAA (SEQ ID NO: 16). In one embodiment, the peptide comprises or consists of a sequence selected in the group comprising or consisting of RMLTDLRAV (SEQ ID NO: 3), LMAQAITGV (SEQ ID NO: 11), VLQDFDQPI (SEQ ID NO: 13), ALMIVSMVV (SEQ ID NO: 14), YIDDILCAA (SEQ ID NO: 16).

In one embodiment, the peptide comprises or consists of an epitope of sequence KLLGDINWI (SEQ ID NO: 1). In one embodiment, the peptide comprises or consists of an epitope of sequence FIFTLIAV1 (SEQ ID N: 2). In one embodiment, the peptide comprises or consists of an epitope of sequence RMLTDLRAV (SEQ ID NO: 3). In one embodiment, the peptide comprises or consists of an epitope of sequence FLSLYFVSV (SEQ ID NO: 4). In one embodiment, the peptide comprises or consists of an epitope of sequence TLIAVIMGL (SEQ ID NO: 5). In one embodiment, the peptide comprises or consists of an epitope of sequence YLDQIATLI (SEQ ID NO: 6). In one embodiment, the peptide comprises or consists of an epitope of sequence FLQAEVANA (SEQ ID NO: 7). In one embodiment, the peptide comprises or consists of an epitope of sequence WMGDRLMSL (SEQ ID NO: 8). In one embodiment, the peptide comprises or consists of an epitope of sequence ALHSSVQSV (SEQ ID NO: 9). In one embodiment, the peptide comprises or consists of an epitope of sequence ILTEVLKGV (SEQ ID NO: 10). In one embodiment, the peptide comprises or consists of an epitope of sequence LMAQAITGV (SEQ ID NO: 11). In one embodiment, the peptide comprises or consists of an epitope of sequence RLSYVHVTV (SEQ ID NO: 12). In one embodiment, the peptide comprises or consists of an epitope of sequence VLQDFDQPI (SEQ ID NO: 13). In one embodiment, the peptide comprises or consists of an epitope of sequence ALMIVSMVV (SEQ ID NO: 14). In one embodiment, the peptide comprises or consists of an epitope of sequence MLLAALMIV (SEQ ID NO: 15). In one embodiment, the peptide comprises or consists of an epitope of sequence YIDDILCAA (SEQ ID NO: 16). In one embodiment, the peptide comprises or consists of an epitope of sequence YIIHYIDDI (SEQ ID NO: 17). In one embodiment, the peptide comprises or consists of an epitope of sequence YIWCPTWRL (SEQ ID NO: 18). In one embodiment, the peptide comprises or consists of an epitope of sequence YIWCPTWSL (SEQ ID NO: 19).

In one embodiment, the peptide comprises or consists of KLLGDINWI (SEQ ID NO: 1). In one embodiment, the peptide comprises or consists of FIFTLIAV1 (SEQ ID N: 2). In one embodiment, the peptide comprises or consists of RMLTDLRAV (SEQ ID NO: 3). In one embodiment, the peptide comprises or consists of FLSLYFVSV (SEQ ID NO: 4). In one embodiment, the peptide comprises or consists of TLIAVIMGL (SEQ ID NO: 5). In one embodiment, the peptide comprises or consists of YLDQIATLI (SEQ ID NO: 6). In one embodiment, the peptide comprises or consists of FLQAEVANA (SEQ ID NO: 7). In one embodiment, the peptide comprises or consists of WMGDRLMSL (SEQ ID NO: 8). In one embodiment, the peptide comprises or consists of ALHSSVQSV (SEQ ID NO: 9). In one embodiment, the peptide comprises or consists of ILTEVLKGV (SEQ ID NO: 10). In one embodiment, the peptide comprises or consists of LMAQAITGV (SEQ ID NO: 11). In one embodiment, the peptide comprises or consists of RLSYVHVTV (SEQ ID NO: 12). In one embodiment, the peptide comprises or consists of VLQDFDQPI (SEQ ID NO: 13). In one embodiment, the peptide comprises or consists of ALMIVSMVV (SEQ ID NO: 14). In one embodiment, the peptide comprises or consists of MLLAALMIV (SEQ ID NO: 15). In one embodiment, the peptide comprises or consists of YIDDILCAA (SEQ ID NO: 16). In one embodiment, the peptide comprises or consists of YIIHYIDDI (SEQ ID NO: 17). In one embodiment, the peptide comprises or consists of YIWCPTWRL (SEQ ID NO: 18). In one embodiment, the peptide comprises or consists of YIWCPTWSL (SEQ ID NO: 19).

Said peptides may be made by any technique known to those of skill in the art, including the expression of proteins, polypeptides or peptides through standard molecular biological techniques, the isolation of proteins or peptides from natural sources, or the chemical synthesis of proteins or peptides. Synthetic peptides will generally be about up 35 residues long, which is the approximate upper length limit of automated peptide synthesis machines, such as those available from Applied Biosystems (Foster City, Calif.). Longer peptides also may be prepared, e.g., by recombinant means.

The present invention also relates to an expression vector inducing expression of one or more peptide(s) as described hereinabove. The present invention also relates to an expression vector comprising a nucleic acid sequence encoding one or more peptide(s) as described hereinabove.

Said vector may be especially a RNA vector, a DNA vector or plasmid, a viral vector or a bacterial vector. There can be integration of an expression cassette into the host cell genome or there can be no integration, depending on the nature of the vector and as this is well known to the skilled person. The expression vector or the expression cassette may further comprise elements necessary for the in vivo expression of the nucleic acid (polynucleotide) in a subject. For example, this may consist of an initiation codon (ATG), a stop codon and a promoter, as well as a polyadenylation sequence for certain vectors such as the plasmids and viral vectors other than poxviruses. The ATG may be placed at 5′ of the reading frame and a stop codon may be placed at 3′. As it is well-known, other elements making it possible to control the expression could be present, such as enhancer sequences, stabilizing sequences and signal sequences permitting the secretion of the peptide.

Regarding RNA vectors, said vectors may use, for example, non-replicating mRNA or virally derived, self-amplifying RNA. Conventional mRNA-based vectors may encode the peptide of interest and may contain 5′ and 3′ untranslated regions (UTRs). Self-amplifying RNAs may encode not only the peptide of interest but also the viral replication machinery that enables intracellular RNA amplification and abundant protein expression.

Examples of viral vectors, include, without limitation, lentivirus and retrovirus.

In one embodiment, the method to obtain the peptides as described hereinabove comprises:
introducing in vitro or ex vivo a vector as described hereinabove into a competent host cell;
culturing in vitro or ex vivo host cells transformed with the expression vector as described hereinabove, under conditions suitable for expression of the peptides;
optionally, selecting the cells which express and/or secrete said peptides; and
recovering the expressed peptides.

The present invention also relates to a cytotoxic T lymphocyte (CTL) of a subject treated with one or more peptide(s) as described hereinabove.

The present invention also relates to a cytotoxic T lymphocyte (CTL) of a subject treated with one or more expression vector(s) as described hereinabove.

The present invention also relates to a T-cell receptor (TCR) recognizing a peptide as described hereinabove.

The present invention also relates to an engineered T cell expressing a TCR recognizing a peptide as described hereinabove.

The process of preparing these T cells is known from the skilled person. It may be the following: (i) TCR α and β chains are isolated from T cells recognizing peptides as described hereinabove and inserted into a vector; (ii) T cells isolated from the peripheral blood of a patient or a donor are modified with such a vector to encode the desired TCRαβ sequences; (iii) these modified T cells are then expanded in vitro to obtain sufficient numbers for treatment and administered into the patient. Of note, TCR sequences can be modified for optimization of TCR affinity.

The present invention also relates to one or more peptide(s), one or more expression vector(s), one or more CTL(s), or one or more engineered T cell(s) as described hereinabove for use as a vaccine.

The present invention also relates to one or more peptide(s), one or more expression vector(s), one or more CTL(s), or one or more engineered T cell(s) as described hereinabove for use as a medicament.

The present invention also relates to one or more peptide(s), one or more expression vector(s), one or more CTL(s), or one or more engineered T cell(s) as described hereinabove for use in treating or preventing a cancer in a subject in need thereof.

The present invention also relates to the use of one or more peptide(s), one or more expression vector(s), one or more CTL(s), or one or more engineered T cell(s) as described hereinabove in the manufacture of a medicament for treating or preventing a cancer in a subject in need thereof.

The present invention also relates to a method for treating or preventing a cancer in a subject in need thereof, wherein said method comprises the administration of one or more peptide(s), one or more expression vector(s), one or more CTL(s), or one or more engineered T cell(s) as described hereinabove in said subject.

In one embodiment, said cancer is selected from the group comprising or consisting of breast cancer, including triple negative breast cancer (TNBC), ovarian cancer, melanoma, sarcoma, teratocarcinoma, bladder cancer, lung cancer, including non-small cell lung carcinoma and small cell lung carcinoma, head and neck cancer, colorectal cancer, glioblastoma, leukemias, lymphomas and other solid tumors and hematological malignancies.

In one embodiment, said cancer is selected from the group comprising or consisting of breast cancer, including triple negative breast cancer (TNBC), ovarian cancer, melanoma, sarcoma, lung cancer, including non-small cell lung carcinoma and small cell lung carcinoma, head and neck cancer, glioblastoma, leukemias, lymphomas and other solid tumors and hematological malignancies.

In one embodiment, said cancer is a breast cancer, preferably TNBC.

In one embodiment, the one or more peptide(s), one or more expression vector(s), one or more CTL(s) or one or more engineered T cell(s) as described hereinabove induce an immune response, such as a T cell response, preferably an immune response against the tumor-associated epitope.

One of ordinary skill would know various assays to determine whether an immune response against a tumor-associated epitope was generated. Various B lymphocyte and T lymphocyte assays are well known, such as ELISAs, cytotoxic T lymphocyte (CTL) assays, such as chromium release assays, proliferation assays using peripheral blood lymphocytes (PBL), tetramer assays, and cytokine production assays.

Thus, the present invention also relates to a method for inducing an immune response in a subject in need thereof, wherein said method comprises the administration of one or more peptide(s), one or more expression vector(s), one or more CTL(s), or one or more engineered T cell(s) as described hereinabove in said subject.

In one embodiment, the one or more peptide(s), one or more expression vector(s), one or more CTL(s), or one or more engineered T cell(s) as described hereinabove is/are administered at a therapeutically effective amount.

It will be however understood that the total daily usage of one or more peptide(s), one or more expression vector(s), one or more CTL(s), or one or more engineered T cell(s) as described hereinabove will be decided by the attending physician within the scope of sound medical judgment.

The specific dose for any particular subject will depend upon a variety of factors including the symptom being treated and the severity of the symptom; activity of the specific compound employed; the specific composition employed, the age, body weight, general health, sex and diet of the patient; the time of administration, route of administration, and rate of excretion of the specific compound employed; the duration of the treatment; drugs used in combination or coincidental with the specific compounds employed; and like factors well known in the medical arts.

For use in administration to a subject, the one or more peptide(s), one or more expression vector(s), one or more CTL(s) or one or more engineered T cell(s) is/are to be formulated for administration to the subject. The one or more peptide(s), one or more expression vector(s), one or more CTL(s) or one or more engineered T cell(s) as described hereinabove may be administered parenterally, by inhalation spray, rectally, nasally, or via an implanted reservoir. The term administration used herein includes subcutaneous, intravenous, intramuscular, intra-articular, intra-synovial, intrasternal, intrathecal, intrahepatic, intralesional and intracranial injection or infusion techniques.

### BRIEF DESCRIPTION OF THE DRAWINGS

FIG. 1 is a combination of diagrams and one histogram showing the pancancer identification of HERVs associated with CTL responses. FIG. 1A: Venn diagram representing the total number of HERVs overexpressed in a tumor versus its normal counterpart (peritumoral tissue), and the total number of HERVs overexpressed in a normal peritumoral tissue versus its tumoral counterpart. HERVs overexpressed in at least 1 tumor and never overexpressed in any peritumoral tissue are considered cancer-associated. FIG. 1B: Venn diagram of the selection criteria for a HERV to be annotated as associated with CTL response (cyt-HERV). Each HERV had to be associated with both a phenotype (CD8 or CD4 T cell signatures) and a function (cytolytic activity (Granzyme B & Perforin 1) or IFN-γ signature) criteria (A and B) and not overexpressed in normal purified T/NK cells (A AND B NOT C). FIG. 1C: Venn diagram of cancer-associated HERVs' association with CTL responses criteria defined in A. A total of 192 HERVs are annotated as cyt-HERVs. FIG. 1D: Proportion of cancer-associated HERVs annotated as cyt-HERVs per cancer subtype. Cyt-HERVs are represented in light grey. CTL: Cytotoxic T cell response, Cyt-HERVs: HERVs associated with CTL response in cancer, TNBC: Triple-Negative Breast Cancer

FIG. 2 is a combination of one histogram, one schema and one table showing the selection of shared HLA-A2 epitopes derived from Gag and Pol HERV-K/HML-2.

FIG. 2A: Flow Chart of peptide selection from cyt-HERVs sequences. FIG. 2B: Bar chart of the top 25 most shared peptides predicted as strong HLA-A*02 binders among the 192 cyt-HERVs. Selected peptides (P1 to P6) are marked with a star. FIG. 2C: Characteristics of the 6 selected HLA-A2 epitopes. 9-mer peptides were selected according to their predicted HLA-A*02 affinity (considering strong binders for percentile ranks ≤0.5) and the number of HERVs containing their sequences.

FIG. 3 is a combination of a diagram and two histograms showing that the shared CD8+ T cell epitopes derived from conserved Gag and Pol HERV-K/HML-2 motifs are expressed in TNBC. FIG. 3A: Venn diagram of total number of cyt-HERVs overexpressed in each subtype of breast cancer in TCGA database. FIG. 3B: Mean expression of the 54 cyt-HERVs overexpressed in TCGA basal subtype, in the independent database of Varley et al and in medullary thymic epithelial cells (mTECs). FIG. 3C: Expression of the 18 peptide-containing CAHs in the breast cancer basal (Hs578t & MDA-MB-231) and luminal A (MCF7 and T47D) cell lines analyzed by Riboseq.

FIG. 4 is a combination of schemas and plots showing that HERV-derived epitopes induce polyfunctional CD8+ T cell responses. FIG. 4A: Schematic representation of the in vitro priming protocol. FIG. 4B: Summary of the results obtained with PBMCs from 11 HLA-A2-positive HD (HD1 to HD11, one donor per line). FIG. 4C: Plots of IFN-γ (left panels), IFN-γ and TNF-α (center panels) or IFN-γ and CD107a (right panels) staining gated on CD8+ T cells. PBMCs were stimulated with peptide (here P6, upper line), no peptide (central line) or CMV pp65 peptide (bottom line).

FIG. 5 is a combination of tables and a diagram showing the visualization of CDR Loops and CDR Loop interactions with peptides. FIG. 5A: Productive frequency of the TCRα and TCRβ CDR3 sequences for the top clones specific to each peptide (P1, P2, P4, P6) and the corresponding resolved V, D and J alleles. FIG. 5B: Predicted binding affinities (Predict. Ag) are expressed in kcal/mol. Average values are reported. Stars indicate significant statistical test (Welch two-sample t-test) at the 5% level. FIG. 5C: Diagram ranking of modeled HERV-specific TCR-pMHC and reference TCR-pMHC complexes available in the Protein Data Bank and obtained from crystallography data, according to their predicted binding affinity. CDR: complementarity-determining region, TCR: T cell receptor; MHC: major histocompatibility complex; pMHC: peptide-MHC, TRA: alpha chain of TCR; TRB: beta chain of TCR.

FIG. 6 is a combination of graphs showing that HERV-specific T cell clones are functional, recognize and kill tumor cells. FIG. 6A: Functional avidity of CD8+ T cell clones calculated as nonlinear fit of normalized IFN-γ production. N9-V1 and -2: CMV-specific T cell clones (see Methods). EC50 are represented for each clone by the interpolation of the dashed lines with the X-axis. FIG. 6B: Cell death quantification represented as fluorescence intensity increase from the baseline (Y-axis) in function of the time (hours, X-axis). FIG. 6C: Specific tumor cell lysis at 48h. Mean percentage of technical triplicates is plotted for each condition (data representative of at least 2 independent experiments).

FIG. 7 is a combination of a graph and pictures showing that HERV-specific T cells are present among tumor infiltrating T cells. FIG. 7A: Overall survival according to 18-HERVs score in TCGA HLA-A2 TNBC patients (n=65). Patients were divided in three groups according to the score terciles: high expression (n=22); intermediate expression (n=21); low expression (n=22). FIG. 7B: 60X-pictures of TNBC organoids co-cultured with CMV, P1 or P6-specific CD8+ T cell clones (top-down) acquired at different time points using Nanolive technology. T cells are shown by white arrows.

### EXAMPLES

The present invention is further illustrated by the following examples.

### Materials and Methods

### Datasets

For RNA-seq data, raw fastq files were accessed from the NCBI Gene Expression Omnibus (GEO) portal, under the accession number GSE58135 for Varley et al. independent breast cancer dataset (K. E. Varley et al., Recurrent read-through fusion transcripts in breast cancer. Breast Cancer Res. Treat. 146, 287-297 (2014)), GSE74246 for the sorted PBMC dataset (M. R. Corces et al., Lineage-specific and single-cell chromatin accessibility charts human hematopoiesis and leukemia evolution. Nature Genetics. 48, 1193-1203 (2016)), GSE127825 and GSE127826 for the six mTECs samples (J.-D. Larouche et al., Widespread and tissue-specific expression of endogenous retroelements in human somatic tissues. Genome Med. 12, 1-16 (2020)). TCGA pancancer raw fastq files were accessed from the Genomic Data Commons (GDC) portal (https://portal.gdc.cancer.gov/). Cell line data were accessed from the Broad Institute Cancer Cell Line Encyclopedia (CCLE) portal (https://portals.broadinstitute.org/ccle).

### HERV Expression Quantification

HERV expression was assessed using the HervQuant pipeline (C. C. Smith et al., Endogenous retroviral signatures predict immunotherapy response in clear cell renal cell carcinoma. Journal of Clinical Investigation. 128, 4804-4820 (2018)). Briefly, RNAseq reads were mapped with STAR v2.7.3a (A. Dobin et al., STAR: ultrafast universal RNA-seq aligner. Bioinformatics. 29, 15-21 (2013)) to the hg19 reference transcriptome compiled with the annotation of 3,173 HERV sequences (L. Vargiu et al., Classification and characterization of human endogenous retroviruses; mosaic forms are common. Retrovirology. 13, 7 (2016)). Multimaps≤10 and mismatch≤7 were allowed, as in the original publication. BAM outputs were filtered for reads that mapped HERV sequences using SAMtools v1.4 (H. Li et al., 1000 Genome Project Data Processing Subgroup, The Sequence Alignment/Map format and SAMtools. Bioinformatics. 25, 2078-2079 (2009)) and then quantified using Salmon v0.7.2 (R. Patro et al., Salmon: fast and bias-aware quantification of transcript expression using dual-phase inference. Nat Methods. 14, 417-419 (2017)). Raw counts were normalized to counts per million total reads and then log 2+1 transformed.

### Quality Check/Sample Filtering

Only primary solid tumor samples (TCGA code 01) were included, regrouping 9,718 samples from 32 different cancer types, from which 9,492 were analyzable for HERV expression. Quality check resulted in complete removal of ESCA and STAD samples due to a largely skewed HERV distribution, leading to the final analysis of 8,893 samples from 29 different cancer types.

### Immune Signatures and Genetic Alterations

Phenotypic immune signatures were calculated with the Xcell method (D. Aran, et al., xCell: digitally portraying the tissue cellular heterogeneity landscape. Genome Biol. 18, 220 (2017)). For the TCGA Pancancer samples, Xcell signatures were directly downloaded from the Xcell website (https://xcell.ucsf.edu/xCell_TCGA_RSEM.txt). For the GSM1401648 dataset, signatures were calculated for the whole dataset, and immune signatures were filtered after. Interferon-gamma (IFN-γ) signature was calculated by single-sample gene set variation analysis (GSVA) (S. Hänzelmann, et al., GSVA: gene set variation analysis for microarray and RNA-Seq data. BMC Bioinformatics. 14, 7 (2013)). based on the HALLMARK_INTERFERON_GAMMA_RESPONSE signature from the Molecular Signature Database (http://software.broadinstitute.org/gsea/msigdb/index.jsp). Enrichment scores were calculated for each sample per cancer type. The cytolytic activity (CYT_score) was calculated as the geometric mean of granzyme-B (GRZB) and perforin (PRF1) expression, as previously described (M. S. Rooney et al., Molecular and genetic properties of tumors associated with local immune cytolytic activity. Cell. 160, 48-61 (2015)). TCGA Pancancer Genetic alterations were retrieved from Thorsson V. et al. (V. Thorsson et al., The Immune Landscape of Cancer. Immunity. 48, 812-830.e14 (2018)).

### Cancer-Associated and Cyt-HERV Annotation

To define cancer-specificity, differential HERV expression was performed between tumor samples and their respective normal peritumoral matched tissues. Only TCGA studies with at least 10 peritumoral samples were included (n=14 different cancer types). Differential HERV expression analysis was performed independently for each TCGA cancer type. Having filtered-out any HERV expressed more than 2-fold in any normal tissue compared to its matched tumor, remaining HERVs overexpressed more than 2 fold in at least one cancer compared to its normal counterpart (peritumoral tissue) was considered cancer-associated.

To be annotated as potentially immunogenic, each cancer-associated HERV had to be associated with at least one phenotype (A) criterion and one functionality (B) criterion and not be overexpressed by T/NK cells (C). Phenotype criteria included association with either CD4 or CD8+ T cell signatures as defined by the Xcell method. Function criteria included association with either IFN-γ or the cytolytic activity, defined by the geometric mean of granzyme-A (GZMA) and perforin (PRF1) expression. Normal PBMC expression was assessed in an independent dataset of sorted-PBMCs from healthy donors (M. R. Corces et al., Lineage-specific and single-cell chromatin accessibility charts human hematopoiesis and leukemia evolution. Nature Genetics. 48, 1193-1203 (2016)). HERV expression was compared independently in T cells and NK cells to the rest of PBMCs.

### L1-Penalized Regression (LASSO)

Association were calculated by Lasso regression using the glmnet and the c060 packages (J. H. Friedman et al., Regularization Paths for Generalized Linear Models via Coordinate Descent. Journal of Statistical Software. 33, 1-22 (2010), M. Sill et al., c060: Extended Inference with Lasso and Elastic-Net Regularized Cox and Generalized Linear Models. Journal of Statistical Software. 62, 1-22 (2014)). Gaussian distribution was considered for the CYT score and the IFN-γ signatures, and Poisson distribution was considered for the Xcell signatures. HERVs were analyzed as log 2(CPM+1), requiring no further standardization. For each cancer type, a model was built based on optimal parameters found with 10-fold cross validation. Each HERV with a positive coefficient in the final model (based on the lambda parameter minimizing the mean-squared error) was considered to be associated with the variable.

### Epitope Screening

Open-reading frame (ORF) detection was performed using sixpack from EMBOSS v6.6.0.0 (F. Madeira et al., The EMBL-EBI search and sequence analysis tools APIs in 2019. Nucleic Acids Res. 47, W636-W641 (2019)). Detected ORFs of more than 10 amino acids were then aligned to known HML-2 (HERV-K) Gag, Pro, Pol, Env, Rec and Np9 proteins referenced in UniProt (UniProt: a worldwide hub of protein knowledge. Nucleic Acids Res. 47, D506-D515 (2019)). Blast with optimal parameters for retrovirus was used (Word size=3, composition-based statistics, no “low-complexity-region” filter). Conserved sequences aligned with Gag and Pol proteins with more than 90% identity and an e-value <0.05 were then screened for predicted HLA-A*02 strong binders using MHCflurry v1.3 (T. J. O'Donnell et al., MHCflurry: Open-Source Class I MHC Binding Affinity Prediction. Cell Systems. 7, 129-132.e4 (2018)). Peptides with a rank<=0.5 percentile were considered to be strong-binders. The human proteome was downloaded on Uniprot (ID: UP000005640) to validate the absence of match before peptide synthesis and in vitro validation.

### Cumulative Expression Score

The T-value score was defined for each HERV and each tissue comparison (TNBC versus peritumoral tissue) as the product of the log 2 fold change of expression and the log 10 of the inverse p-value, according to the method proposed by Xiao et al (Y. Xiao et al., A novel significance score for gene selection and ranking. Bioinformatics. 30, 801-807 (2014)). The cumulative expression score was calculated by summing the T-values of all the HERVs containing the epitope sequence (including CAHs and other HERVs).

### Analysis of Peptidome Proteomic Datasets

Raw MS/MS datasets were downloaded from CPTAC (N. J. Edwards et al., The CPTAC Data Portal: A Resource for Cancer Proteomics Research. J Proteome Res. 14, 2707-2713 (2015)) for breast cancer studies (Cancer Genome Atlas Network, Comprehensive molecular portraits of human breast tumours. Nature. 490, 61-70 (2012); K. Krug et al., Proteogenomic Landscape of Breast Cancer Tumorigenesis and Targeted Therapy. Cell. 183, 1436-1456.e31 (2020)). Retrieved MS/MS spectra were converted to MGF format using msconvert from proteowizard (R. Adusumilli, P. Mallick, Data Conversion with Proteo Wizard msConvert. Methods Mol Biol. 1550, 339-368 (2017)). Then the list of peptides was analysed using the standalone version of Pepquery (v.1.6.2.0) (B. Wen et al., PepQuery enables fast, accurate, and convenient proteomic validation of novel genomic alterations. Genome Res. 29, 485-493 (2019)). The used command line was: java-Xmx10G-jar pepquery.jar-fixMod 6,62,108-varMod 117-max Var 3-c 1-tol 10-tolu ppm-minScore 12-e 1-um-hc TRUE-n 1000-itol 0.05-m 1 cpu 12-pep $ {peptides_list}-db $ {Reference_database}-ms $ {MS_database}-0 $ {output_directory}. For the second data set the fixmod and varMod in the command line were adapted like the following: -fixmod 6,103,157 -varMod 101,117.

### Riboseq Analysis

Ribosome-profiling data were retrieved from a previously published study (F. Loayza-Puch et al., Tumour-specific proline vulnerability uncovered by differential ribosome codon reading. Nature. 530, 490-494 (2016)). Raw fastq files were pre-processed as described in the initial publication. Briefly, adapter sequences were trimmed from raw data using cutadapt 1.1 with parameters (-quality-base=33-O 12-m 20-q 5) and mapped to our reference hg19-HERV transcriptome.

### Statistical Analysis

All analyses were performed using R statistical software version 3.6.0. Differential HERV expression analysis was performed using DESEQ2 v1.24.0 (M. I. Love et al., Moderated estimation of fold change and dispersion for RNA-seq data with DESeq2. Genome Biol. 15, 550 (2014)) and logarithmic fold changes were shrunk with the apeglm package (A. Zhu et al., Heavy-tailed prior distributions for sequence count data: removing the noise and preserving large differences. Bioinformatics. 35, 2084-2092 (2019)).

### Biological Samples

Blood from healthy donors was obtained from the “Etablissement Français du Sang” (Lyon). Fresh TNBC (n=11) were provided by the tissue bank of Centre Léon Bérard (CLB) (BB-0033-00050, CRB-CLB, Lyon, France; French agreement number: AC-2013-1871), after approval from the institutional review board and ethics committee (L-06-36 and L-11-26) and patient written informed consent, in accordance with the Declaration of Helsinki.

### Peptides Synthesis

Peptides were synthetized at JPT peptide Technologies (GE, EU) with a specification and a purity >90%. Lyophylized powder was resuspended in 1% DMSO distilled water.

### Cell Lines

MDA-MB-231 basal breast cancer epithelial cells were obtained from American Type Culture Collection (ATCC catalog name: HTB-26) and cultured in 10% FBS DMEM (Gibco, FR, EU) 1% Penicillyn/streptomicyn 1% L-Glutammine. HMEC primary cells were obtained from Promocell (GE, EU) and cultured in mammary epithelial growth medium (Promo Cell, GE, EU).

### In Vitro Priming Assays

PBMCs were obtained by Ficoll density gradient centrifugation (Eurobio, FR, EU). They were rapidly thawed at 37° C. and extensively washed, let at room temperature or overnight at 37° C. before assessing their viability. 0.15×10⁶ PBMCs per well were cultured in 96 well plates with AIM V Medium (Gibco, FR, EU) enriched with 5 μg/mL R-848 (Resquimod), 10 μg/mL HMW poly-IC (both Invivogen, FR, EU), 20 IU/mL IL-2 (PROLEUKIN aldesleukine, Novartis Pharma, CH, EU) and 10 μg/mL of the peptide of interest at day 0. After 3, 6 and 10 days 100 μL of medium were replaced by enriched fresh medium (IL-2 and peptide only at day 6 and IL-2 only at day 10) and splitted if necessary. On day 12 cells were collected and counted for analysis.

### Feeding Protocol

Dextramer single-cell sorted CD8+ T cells were expanded on a feeder composed by 35 Gy-irradiated allogeneic PBMCs and B-lymphoblastoic cell lines in a ratio 10:1. Feeder cells were plated in a 96-well round bottom plate at a concentration of 0.10×10⁶ cells per well in RPMI 5% human serum with PHA-L 1.5 μg/mL (Merck KgAa, GE, EU) and IL-2 150 IU/mL (Novartis Pharma, CH, EU) and up to 5×10³ sorted cells were added per well. Cells were cultured for 14 days and medium was replaced when needed with fresh IL-2 enriched RPMI 5% human serum. This process was repeated if needed.

### TCR Immunosequencing

DNA from specific CD8+ T cells and the corresponding bulk PBMCs was extracted using the QIAGEN QIAmp® DNA Blood Micro kit (QIAGEN, GE, EU) and sent for TCR survey and deep analysis to Adaptive Biotechnologies (WA, US).

### Generation and Refinement of 3D Models

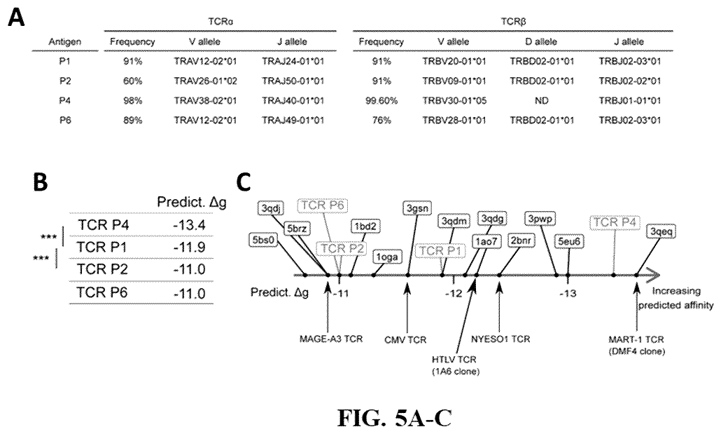

The full TCR sequences of both alpha and beta chains were reconstructed for each T cell clone from the results of the immunosequencing as previously published (A. Gros et al., Recognition of human gastrointestinal cancer neoantigens by circulating PD-1+ lymphocytes. J Clin Invest. 129, 4992-5004 (2019)) For variable domains, TRA and TRB CDR3 nucleotide sequences were obtained from immunosequencing (FIG. 5A) and the 5′ and 3′ ends of the TRAV and TRBV regions were obtained from International Immunogenetics Information System (IMGT) online database. Human constant domains of TRA and TRB were added in 3′ of the variable domains to reconstitute the full-length TCR. These full-length TCR sequences, together with the MHC and peptide sequences were submitted to the CBS TCRpMHCmodels-1.0 web server, specifically developed for the automatic structural modeling of TCR-pMHC complexes (K. K. Jensen et al., TCRpMHCmodels: Structural modelling of TCR-pMHC class I complexes. Sci Rep. 9, 14530 (2019)) using template-based modeling. TCR residues are renumbered using a standardized procedure (K. R. Abhinandan, A. C. R. Martin, Analysis and improvements to Kabat and structurally correct numbering of antibody variable domains. Mol Immunol. 45, 3832-3839 (2008); M. S. Klausen et al., LYRA, a webserver for lymphocyte receptor structural modeling. Nucleic Acids Res. 43, W349-355 (2015)). The initial models generated by the web server were further refined in four rounds using a protocol adapted from Bobisse et al (S. Bobisse et al., Sensitive and frequent identification of high avidity neo-epitope specific CD8+ T cells in immunotherapy-naive ovarian cancer. Nat Commun. 9, 1-10 (2018)). Briefly, the CDR loops were refined by pairs using Modeller software version 9.25 (A. Fiser, A. Sali, Modeller: generation and refinement of homology-based protein structure models. Methods Enzymol. 374, 461-491 (2003); A. Sali, T. L. Blundell, Comparative protein modelling by satisfaction of spatial restraints. J Mol Biol. 234, 779-815 (1993)): CDR loops α1/α2 at round 1, α1/α3 at round 2, β1/β2 at round 3 and β1/β3 at round 4. Only the residues in coil conformations in the initial models were refined. In each round, 500 models were generated and the best model based on the Modeller internal DOPE score was selected and used as input for the next round.

At the end of the four rounds, a representative model was chosen based on the consensus of unweighted contacts as follows: contacts between residues at the TCR-pMHC interface (defined by a distance lower than 5 Angstrom between heavy atoms) were counted in the set of 4*25 models with best DOPE scores of each refinement round. Then, among the 25 models with best DOPE scores in the final round, the model with the highest number of recurrent contacts, referred as un-normalized CONSRANK score (G. Launay et al., Evaluation of CONSRANK-Like Scoring Functions for Rescoring Ensembles of Protein-Protein Docking Poses. Front Mol Biosci. 7, 559005 (2020); R. Oliva et al., Ranking multiple docking solutions based on the conservation of inter-residue contacts. Proteins. 81, 1571-1584 (2013)), is elected as the representative model.

A quantitative view of potential stabilizing interactions between TCR and pMHC is provided by the frequencies of inter-residue contacts observed in the set of 4*25 models with best DOPE scores of each refinement round. Whereas CDR1 and CDR3 loops of both TCRα and TCRβ chains interact with both the MHC and the peptide, the CDR2 loops interact mostly with the MHC molecule.

### 3D Structure Analysis

The structural similarity between representative models of different complexes was assessed by the RMSD between backbone CDR loops computed with UCSF Chimera (E. F. Pettersen et al., UCSF Chimera—a visualization system for exploratory research and analysis. J Comput Chem. 25, 1605-1612 (2004)). As expected, structural variability was highest within CDRβ3 loops. UCSF Chimera was also used for hydrogen bonds and hydrophobic contact detection and structure visualization.

### Binding Affinity Prediction

The binding affinity was predicted using the prodigy method (A. Vangone, A. M. Bonvin, Contacts-based prediction of binding affinity in protein-protein complexes. Elife. 4, e07454 (2015)) which uses a linear model based on the number and types of contacts at the interface. For each complex, instead of running one prediction on the representative model, we averaged the predictions obtained for the 25 models with best DOPE scores obtained at round 4 of the refinement protocol.

### Fluorospot

After co-culture of PBMCs or CD8⁺ T cells with T2 cells pulsed or not with the peptide in a ratio 10:1 in AIM V medium (Gibco, FR, EU) at 37° C. 9% CO2 for 24 hours, double-color Fluorospot (CTL GmbH, CA, US) with IFN-γ AF488 and Grz-b CTL-red was performed according to manufacturer's instructions. Revelation plate was read on ImmunoSpot® S6 ULTIMATE UV Image Analyzer and analyzed with the ImmunoSpot® analysis software.

### Functional Avidity

Dextramer-isolated specific CD8+ T cells for the selected peptides (P1, P2, P6) were used in a functional avidity IFN-γ production test by enzyme-linked immunosorbent assay (IFN-γ ELISA, Thermo Fisher scientific, FR, EU). Two CMV T cell clones specific for the immuno-dominant epitope N9V (NLVPMVATV) were used. N9V-1 corresponds to pp65 dextramer-selected CD8+ T cells and N9V-2 is a CD8+ T cell clone kindly provided by Dr Henri Vie. Functional avidity of specific CD8+ T-cell responses was assessed by performing limiting peptide dilutions from 10-4 to 10-9M (log) charged on T2 cells pulsed for 5 hours. After wash, peptide-pulsed T2 cells were co-cultured with specific CD8+ T cell in a ratio 1:1 in AIM-V medium (Gibco, FR, EU) supplemented with 5% of human serum. After 18 hours, supernatants were collected and ELISA was performed. The peptide concentration required to achieve a half maximal cytokine response (EC50) was determined (graphpad prism, version 6.0 for Windows was used for the 50% EC (EC50) determinations, R>0.98)

### Epitope Validation and Quantification by MS

Epitope validation and quantification by MS was performed by Complete Omics Inc. (Maryland, USA) according to the method previously described (J. Douglass et al. Bispecific antibodies targeting mutant RAS neoantigens. Sci Immunol. 6, eabd5515 (2021)) with further modifications. In brief, a total of 300 million cells were lysed and peptide-HLA complexes were immunoprecipitated using self-packed Valid-NEO neoantigen enrichment column pre-loaded with anti-human HLA-A, B, C antibody clone W6/32 (Bio-X-Cell). After elution, dissociation, filtration and clean up, peptides were lyophilized before further analysis. Transition parameters for each epitope peptide were examined and curated through Valid-NEO method builder bioinformatics pipeline to exclude ions with excessive noise due to co-elution with impurities and to boost up the detectability through recursive optimizations of significant ions. Absolute copy numbers of peptides presented on the cell surface were calculated based on the quantification using the heavy isotope labeled peptides. The MS data have been deposited via ProteomeXchange and can be accessed through identifier PASS01698.

### Live Imaging

Cells were plated in DMEM (Gibco, FR, EU) medium, 10% SVF, 1% Penicillin/Streptomycin. For IncuCyte analysis medium was removed from the 96 wells plate after overnight cell adhesion. A blocking HLA-A2 antibody (GeneTex, clone BB7.2, GTX75806, CA, US) was added in AIM-V medium (Gibco, FR, EU) for 1 hour, according to conditions. T cells were then added in an effector to target (E:T) ratio 2:1 in the presence of IncuCyte Cytotox dye (Essen Bioscience, UK, EU) for cell death quantification. A 48-hour live imaging was performed at 37° C. 5% CO2 with Incucyte Zoom. Cell death was calculated as the total number of counted stained cells corrected by the number of counted stained cells at baseline. Maximum killing was established using

DMSO. Specific lysis was calculated according to the following formula:

$$\% specific lysis = ( ( ( HERV - specific T cells induced target cell death - spontaneous target cell death ) - ( non - specific dextramer - negative t cells induced target cell death - spontaneous target cell death ) ) / ( DMSO induced target cell death - spontaneous target cell death ) ) \times 100$$

For Nanolive imaging T cells were then added with an E:T 10:1 and phase imaging was performed every minute using Nanolive microscope 3D cell explorer.

### Tumor Dilacerations: Organoids and TILs Expansion

Tumor tissues were dissected into fragments of approximately 1 mm³ and dilacerated with collagenase IV and DNAse for 45 minutes in 20% SVF enriched RPMI. The tumor lysate was centrifuged at 1500 rpm for 5 minutes and resuspended in 5% human serum enriched RPMI. Cells were counted and plated at a density of 5×10⁴ cells per well in a flat bottom 96-well plate with anti-CD3 anti-CD28 Dynabeads (Dynabeads, Gibco, EU) and IL-2 at 100 IU/mL in a ratio beads to cells of 1:4.

For organoids, a part of the tumor lysate (3 to 10 million of cells) was resuspended in 10 mL of Advanced DMEM/F12 medium. Cells were centrifuged at 500 rcf for 10 seconds and then resuspended in full medium. This protocol was repeated 3 to 5 times according to the cell number at the beginning to enrich the cell suspension in epithelial cells. These cells were then cultured according to the protocol previously described by Drehuis et al. (E. Driehuis et al., Establishment of patient-derived cancer organoids for drug-screening applications. Nature Protocols. 15, 3380-3409 (2020)).

### Multi-Parametric Flow Cytometry

T cells were counted and co-cultured with T2 cells loaded or not with the cognate peptide in a 5:1 ratio. After one hour CD107a antibody (BD, clone H4A3) was added in each well with Golgi plug (1/1000) (10 μg/mL, BD, FR, EU). After 5 hours, viability, surface and intra-cellular staining were performed. To assess cytokine expression in CD8+T cells an intracellular staining with the FoxP3 Fixation and Permeabilization kit (Thermo Fisher scientific, Life Technologies, CA, US) was used, according to manufacturer's instructions.

Dextramer staining was performed on PBMCs after a 12-day culture (priming protocol) or on TILs expanded for 14 days after tumor dilaceration. Cells were washed in 2 mL washing buffer (PBS+2% FBS+2 mM EDTA (Sigma Alderich, MI, US)) and stained for 10 minutes with dextramers (Immudex ApS, DK, EU) at room temperature prior to viability and surface marker staining. Washing was performed 2 times to avoid non-specific dextramer staining. CMV pp65 NLVPMVATV was used as positive control. For TILs analysis, a dextramer complexed to a non-natural irrelevant peptide (ALIAPVHAV) was used as negative control.

All samples were analyzed on a LSR-Fortessa (BD Biosciences, FR, EU) with conserved settings throughout the entire study. Data were analyzed using FlowJo Software (Tree Star v10.4, NJ, USA).

### Results

A Machine Learning-Based Approach Allows the Identification of HERVs Associated with CTL Response

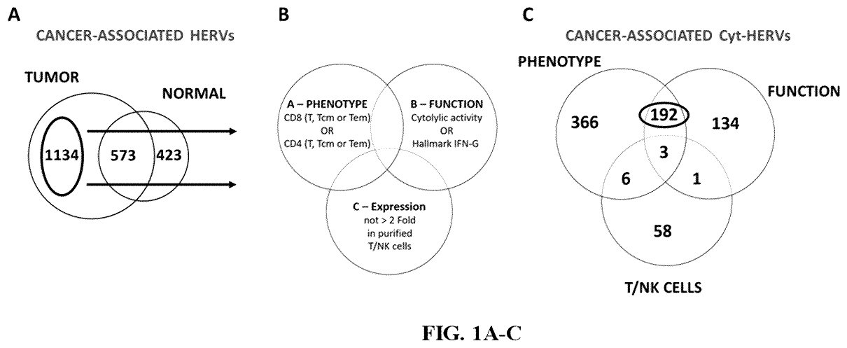

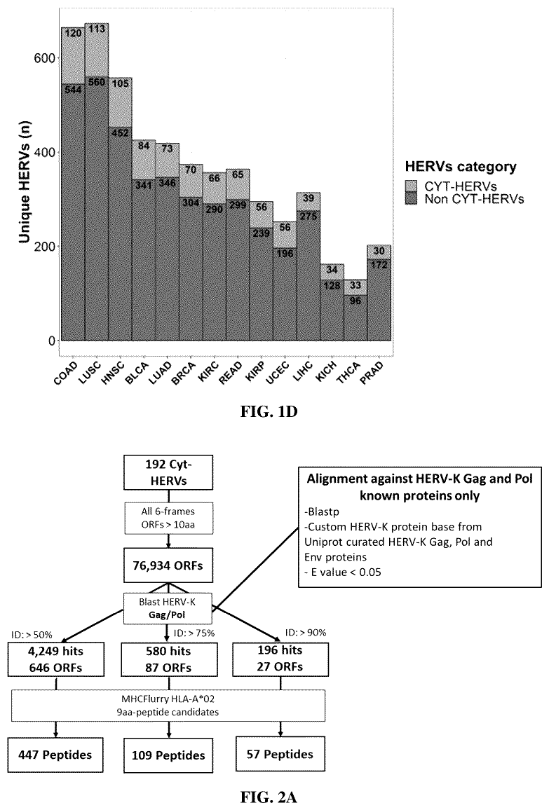

To optimize the epitope detection, we developed a new pipeline for annotating HERVs. For this, we reviewed multiple HERV databases and selected a recent and complete reference of 3,173 HERVs, mostly composed of complete proviral sequences, thus having a higher probability of containing translated peptides. We assessed HERV expression in 8,893 primary tumor samples from 29 different cancer types from The Cancer Genome Atlas (TCGA) pancancer RNAseq database using HervQuant. We selected cancers with at least 10 available matched peritumoral samples (n=14) to filter HERVs highly expressed in tumors and not in normal tissue (cancer-associated HERVs or CAHs). Differential HERV expression analysis unveiled 1,134 CAH candidates (FIG. 1A). To reduce the number of candidates to test, we next applied a second filter to retain only HERVs associated with a CTL response among CAHs (cyt-HERVs). Cyt-HERV annotation was based on two inclusion criteria, namely the association of each HERV with at least one CD4 or CD8 T cell phenotype (A) and function (B) signature, and one exclusion criterion, namely its expression by purified T or NK cells (C) (A and B not C) (FIG. 1B). To reduce the risk of false-positive association and control for the high collinearity encountered with HERV expression, these associations were evaluated by L1 penalized regression (R. Tibshirani, Regression Shrinkage and Selection via the Lasso. Journal of the Royal Statistical Society. 58, 267-288 (1996)) to retain only HER Vs highly associated with CTL responses, controlling for cancer subtypes. A machine learning-based approach was used to test the associations independently for each cancer type (see Methods for full details) leading to the final identification of 192 cyt-HERVs (FIG. 1C). Sub-cancer analysis revealed that colon adenocarcinoma (COAD), lung squamous cell carcinoma (LUSC), head and neck squamous cell carcinoma (HNSC), bladder urothelial carcinoma (BLCA) and lung adenocarcinoma (LUAD) were the top 5 cancers with the highest total number of cyt-HERVs (FIG. 1D).

Overall, cyt-HERVs constituted around 15% of total CAHs, greatly reducing the number of potential candidates. Among the most shared cyt-HERVs, 11 were overexpressed in more than 10 different types of cancers, including 3 HERVs (herv_2256, herv_6069 and herv_4700) formerly reported to induce CD8+ T cell responses. Analysis of the mean beta-value of the 10 nearest surrounding probes from TCGA Illumina 450k methylation data revealed more cyt-HERVs significantly correlated with local demethylation (n=37) than methylation (n=15), suggesting a partial epigenetic control of these HERVs.

### Selection of Conserved Gag and Pol HERV-K/HML-2 Motifs Among Cyt-HERVs Leads to the Identification of Shared CD8+ T Cell Epitopes

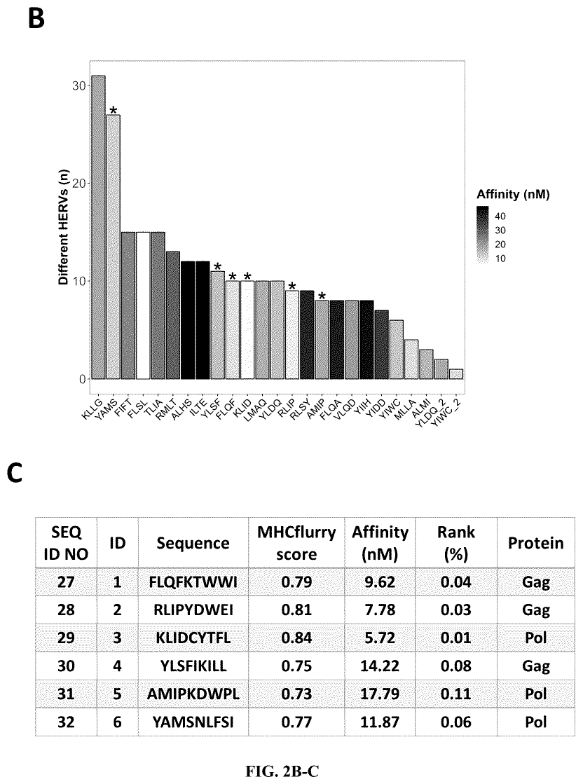

We next assessed the presence of shared T cell epitopes among these cyt-HERVs, focusing on HLA-A2, the most common HLA class I allele. We translated our 192 cyt-HERV sequences into the 6 possible frames and retained predicted open-reading frames (ORFs) of at least 10 amino acids. To reduce the number of false-positives (non-translated sequences), we aligned these ORFs against known HERV-K/HML-2 Gag and Pol proteins referenced in UniProt and kept only ORFs with 90% homology with known existing HML-2 proteins. This conservative approach led to the identification of 57 HML-2 HLA-A*0201 epitope candidates from 27 distinct ORFs (FIG. 2A) with herv_2410 and herv_6069 showing the highest number of conserved HML-2-derived ORFs. To better appreciate the distribution of these epitopes, we relocated each peptide among all the CAHs. The top 25 most shared epitopes are shown in FIG. 2B. Thirteen unique epitopes were present in at least 10 different HERVs (FIG. 2B). For further biological validation and immunological assays, we selected 6 of the most shared epitope candidates: 3 from Gag (P1, P2 and P4) and 3 from Pol (P3, P5 and P6) (FIG. 2C). Analysis of mass spectrometry (MS) data from TCGA and Clinical Proteomic Tumor Analysis Consortium (CPTAC) showed evidence of translation for P1, P2, P3, P5 and P6 peptides. P4 was also selected as it had been described among HLA-I eluted peptides from tumors (patent WO2019/162110A1). Importantly, alignment against the human proteome revealed that the sequences of these epitope candidates did not match any self-protein sequence. Of note, these HLA-A2 epitopes were not predicted as strong binders for the other most common HLA-A and B alleles.

### Triple Negative Breast Cancer (TNBC) is Characterized by Many Cyt-HERVs Containing Shared HERV Epitopes

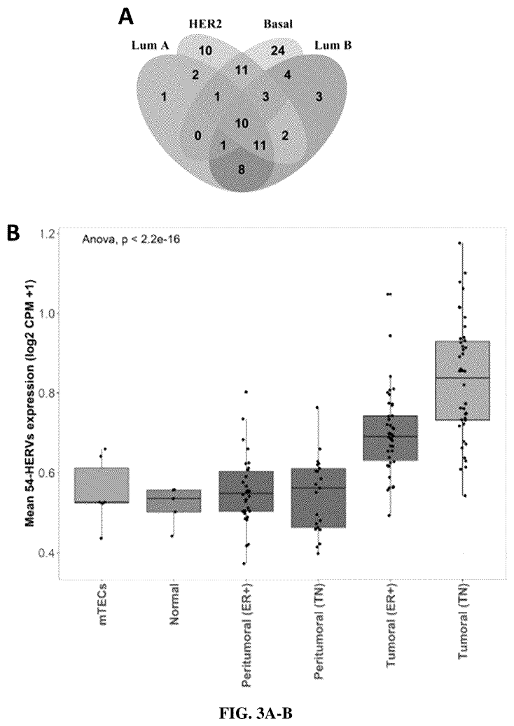

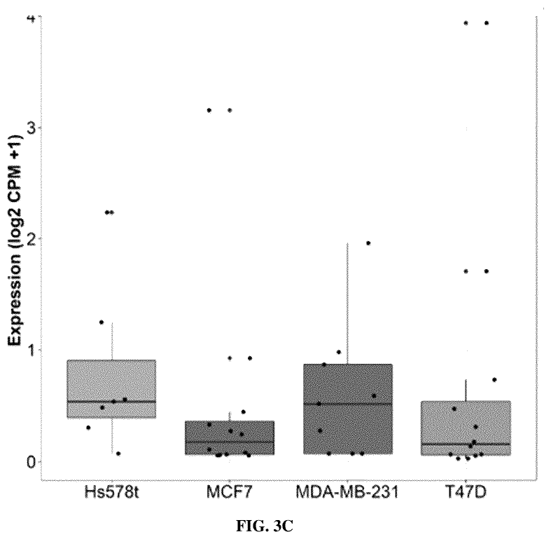

Owing to the well-characterized expression of HERVs in TNBC and the availability of a RNA sequencing (RNAseq) database comprising normal samples, we then focused on breast cancer. Differential HERV expression analysis uncovered a total of 497 CAHs expressed across different breast cancer subtypes, among which 91 were annotated as cyt-HERVs. Fifty-four of these 91 cyt-HERVs were expressed in the basal subtype (FIG. 3A). The mean expression of these 54 cyt-HERVs was significantly higher in TNBC and ER+ samples compared to peritumoral or normal breast tissues from an independent dataset (FIG. 3B). We confirmed the high expression of these 54 cyt-HERVs in breast cancer cell lines sequenced in Varley et al.'s study and in cell lines from the Broad Institute Cancer Cell Line Encyclopedia. The top 25 most shared epitope candidates among all CAHs expressed in the basal subtype contained the 6 previously identified peptides P1-P6.

We next selected HERVs containing the sequences of the 6 previously identified epitope candidates P1-P6 among the CAHs expressed in the basal subtype. Eighteen different CAHs contained at least one of the 6 peptides in their ORFs (Table 1).

TABLE 1

| herv_id | P1 | P2 | P3 | P4 | P5 | P6 |
|---|---|---|---|---|---|---|
| herv_2953 | | | | | | x |
| herv_4833 | x | x | | | | x |
| herv_3232 | | | | | | x |
| herv_4873 | | | x | x | x | x |
| herv_6069 | x | x | x | x | x | x |
| herv_2025 | x | x | x | x | x | x |
| herv_6079 | | x | x | x | x | x |
| herv_2704 | x | x | x | x | x | x |
| herv_1741 | | | x | x | | x |
| herv_3192 | x | x | | x | | x |
| herv_3288 | | | | | | x |
| herv_2794 | | | | | x | |
| herv_2288 | | | | | | x |
| herv_2582 | x | x | x | x | x | x |
| herv_4679 | | | | | | x |
| herv_2476 | | | | | | x |
| herv_4695 | x | x | x | x | x | x |
| herv_3652 | x | x | x | | x | x |

Genomic mapping of the corresponding loci showed a diffuse location for these 18 HERVs on chromosomes. To quantify the differential expression of the epitope-containing HERVs in TNBC versus normal tissues (represented here by each available peritumoral sample), we used the x-value score that takes into account both statistical significance, given by the p-value, and biological significance expressed by the fold change. A cumulative expression score was then calculated for each epitope by summing the x-values of all the HERVs containing its sequence. This score was between 10 and 200 in most cases, which confirmed the significant overexpression of the epitope-containing HERVs in TNBC versus each evaluated normal sample. Finally, analysis of ribosome profiling (riboseq) data from a previously published study revealed evidence of translation for the 18 peptide-containing CAHs in 4 different breast tumor cell lines including 2 basal and 2 luminal A subtypes (FIG. 3C).

Overall, our bioinformatics approach allowed us to select a limited number of HERV-derived T cell epitopes specifically overexpressed by tumor cells and most likely to be immunogenic among a large number of HERV candidates.

### HERV-Derived Epitopes Induce Strong and Polyfunctional T Cell Responses

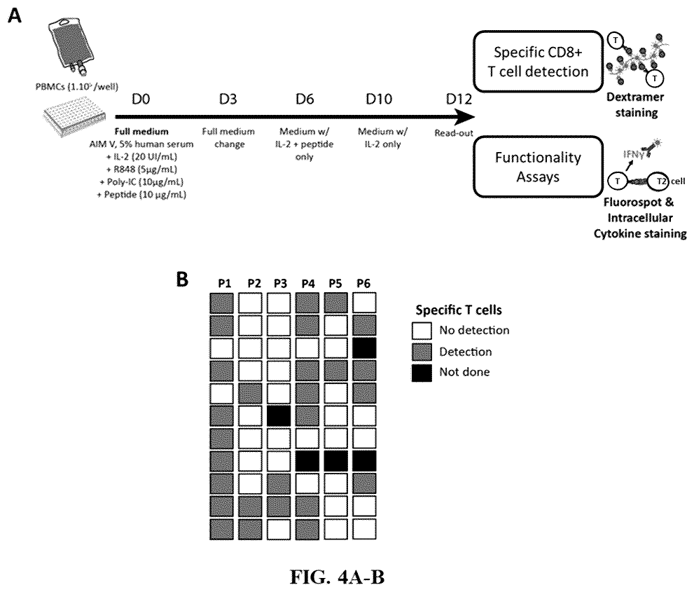

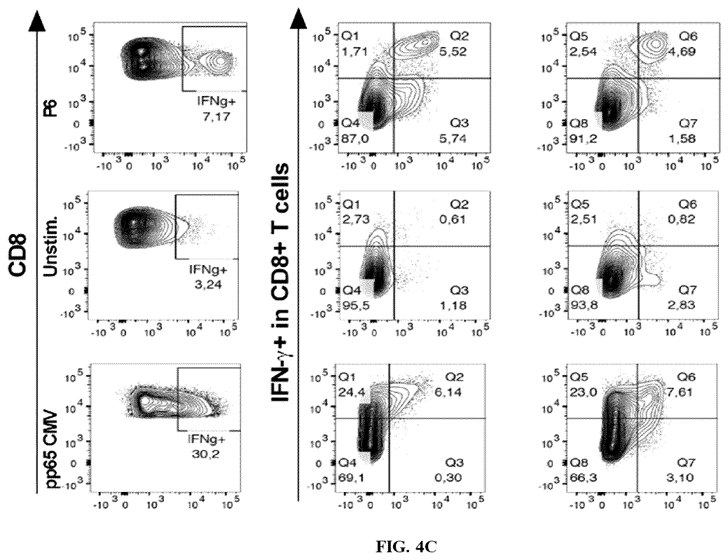

We then evaluated the capacity of the selected epitope candidates to induce efficient T cell responses. The HLA-A2 affinity of the 6 selected peptides was first confirmed using an in vitro binding assay on purified HLA-A*02:01 molecules. To assess the immunogenicity of these 6 peptides, we developed an optimized in vitro priming assay performed on peripheral blood mononuclear cells (PBMCs) from HLA-A2-positive donors (FIG. 4A and Methods for details). The dextramer-based quantification of peptide-specific CD8+ T cells revealed the presence of specific T cells for all peptides, with variations among donors (FIG. 4B). P1 appeared to be the most immunogenic peptide with significant T cell responses in 9/11 donors, followed by P4 (7/10), P6 (4/9) and P2 (3/11) (FIG. 4B). The immunogenicity of these peptides was further confirmed by a classical assay using monocyte-derived dendritic cells (MoDCs) prepared from 5 HLA-A2-positive healthy donors. Flow cytometry analysis showed a CD8+ T cell IFN-γ production when peptide-stimulated PBMCs were co-cultured with T2 cells pulsed with the cognate epitopes. Of note, P1 also induced the highest IFN-γ response compared to the other peptides. In agreement with the bioinformatics prediction, no specific T cell induction was observed using PBMCs from HLA-A2 negative donors (n=5).

Based on these results, we selected P1, P2, P4 and P6 for further experiments. A polyfunctional IFN-γ+ TNF-α+-specific CD8+ T cell response was observed upon co-culture of stimulated PBMCs with peptide-pulsed T2 cells, associated with the presence of the degranulation marker CD107a (FIG. 4C). Fluorospot assay in the same co-culture conditions confirmed the secretion of IFN-γ and granzyme B with the presence of double-positive cells.

### Epitope-Specific CD8+ T Cell Clones are Characterized by T Cell Receptors (TCR) of High Predicted Affinity

P1, P2, P4 and P6-specific CD8+ T cells were sorted by flow cytometry using dextramer staining and expanded on feeder cells (see Methods). More than 90% (90-99%) of the CD8+ T cells were dextramer-positive after one (P1) or two steps (P2, P4 and P6) of selection-expansion. TCRβ immunosequencing confirmed the presence of dominant clones with a unique VB rearrangement representing 90.8%, 90.7%, 99.6% and 76% of the expanded T cells for P1, P2, P4 and P6, respectively (FIG. 5A). Of note, the V/D/J recombination sequences of TCRβ characterizing these clones were not present in the T cell bulk before peptide stimulation (threshold sensitivity: 3×10−6). TCRα chains were also sequenced and confirmed the presence of a unique major clone for P1, P4 and P6, enabling TCR pairing and modeling. Because 2 major Vα rearrangements were obtained for P2, the predominant rearrangement occurring at a 60% frequency was used for TCR modeling.

The affinity of the T cell clones specific for the peptides P1, P2, P4 and P6 was then characterized by considering 3D models of the TCR-peptide-MHC (pMHC) complexes (see Methods). The stability of macro-molecular complexes is due to the formation of favorable interactions at the interface such as hydrogen bonds, salt bridges and hydrophobic interactions. These interactions involve specific side-chains of the peptides that are exposed at the TCR-pMHC interface. In the TCR P1 complex, Phe1, Phe4 and Trp8 side chains of the peptide form several hydrophobic interactions, and the backbone atoms of Phe 4 and Ile 9 are involved in H-bonds. In the TCR P2 complex, several hydrophobic interactions are mediated by peptide residues Pro4, Tyr5 and Trp7. In TCR P4, peptide residues Ile5, Ile7 and Leu8 form several hydrophobic interactions, while Tyr1 and Lys6 side chains, as well as Phe4 and Leu8 backbone atoms are involved in H-bonds. In TCR P6, Tyr1, Ser4, Asn5, Leu6 and Phe7 form several hydrophobic interactions, while Ser4/Leu6/Ser8 backbones and Tyr1/Ser4/Asn5/Ser8 side-chains form 8 H-bonds.

Overall, this analysis of the predicted 3D models suggests that the TCR-pMHC complexes are stabilized by several favorable non-covalent interactions, supporting that the TCR identified after clonal expansion of HERV-specific T cells form a stable complex with the peptides presented by HLA-A2 molecules. To gain further insight, we submitted the 3D models to binding affinity prediction (FIG. 5B). When compared to reference TCR-pMHC complexes available in the Protein Data Bank and obtained from crystallography data, the predicted affinities of the identified TCRs match clinically relevant TCR affinities, such as TCRs targeting MAGE-A3, NY-ESO-1, MART-1, HTLV or CMV (FIG. 5C). Hence, the HERV-specific TCRs identified are predicted to stably interact with their respective pMHC complexes, reminiscent of high affinity TCRs.

### High Avidity HERV-Specific T Cell Clones Recognize and Kill Tumor Cells

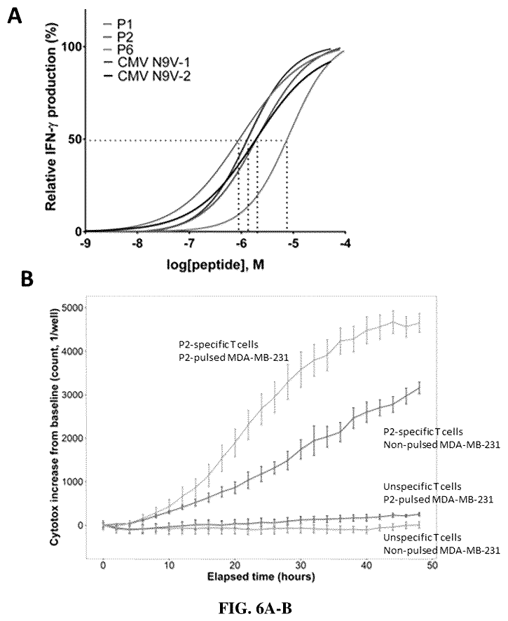

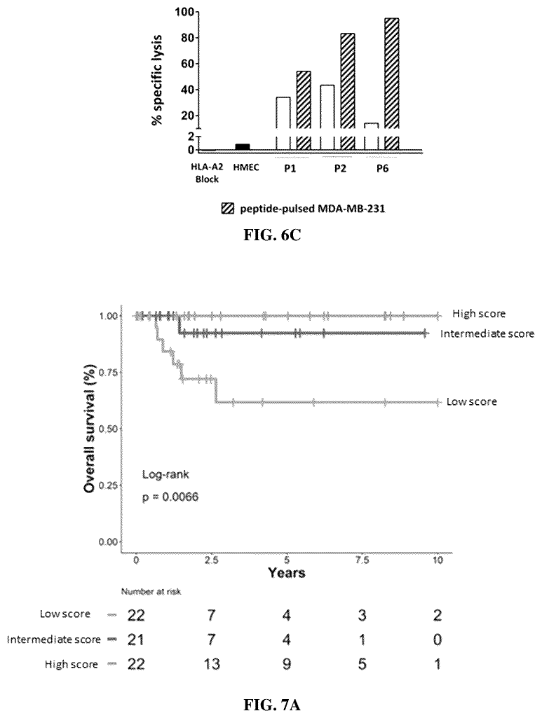

The functionality of the sorted and expanded epitope-specific CD8+ T cells was confirmed by Fluorospot using peptide-pulsed T2 cells. The functional avidity was subsequently assessed by loading T2 cells with decreasing concentrations of the cognate peptide (ranging from 10−4 to 10−9 M) and measuring the lowest peptide concentration necessary to provoke IFN-γ responses in 50% of cells (defined as half maximal effective concentration, i.e. EC50). The EC50 values, estimated at 6.6×10−7 M, 1.9×10−6 M and 6.8×10−6 M for P1, P2 and P6-specific T cells, respectively, are in the same order of magnitude as neoepitope-specific T cell clones (28) and CMV-specific T cells (1.2×10⁻6 and 1.9×10⁻⁶ for N9V-1 and N9V-2, respectively) (FIG. 6A).

We next assessed the capacity of these HERV epitope-specific CD8+ T cells to recognize and kill tumor cells. We selected as a target candidate the HLA-A2-positive MDA-MB-231 basal BRCA tumor cell line, previously shown to express HERVs containing epitope sequences (FIG. 3C). To provide evidence that the epitopes are actually presented on the cell surface, a MS-based method was used to analyze peptides eluted from HLA molecules. P1 and P6 epitopes were clearly detected by MS. On the basis of comparison with the heavy isotope-labeled control, we estimated that there were 1.8 copies of P1-HLA complexes on MDA-MB-231 cell surface.

Tumor cells were co-cultured with the epitope-specific T cells or with the dextramer-negative CD8+ T cell fraction sorted and expanded in the same conditions (negative controls). Flow cytometry analysis highlighted IFN-γ production by approximately 25% of epitope-specific T cells in contact with MDA-MB-231, with a significant increase (>6-fold) compared to the background observed with non-specific T cells. This IFN-γ production was inhibited by a HLA-A2 blocking monoclonal antibody, demonstrating that the T cell clones specifically recognized the tumor cells in a HLA-A2 restricted manner.

In order to monitor tumor cell death in real-time, we performed an immune-cell killing assay using the IncuCyte technology. T cell clones induced a significant and HLA-A2-restricted killing of MDA-MB-231 cells, as shown by the time-dependent increase in the amount of Cytotox fluorescent reagent of target cells. In contrast, the dextramer-negative fraction of T cells did not induce significant cell death of MDA-MB-231 cells (pulsed or not with the peptide) (FIG. 6B). Specific lysis (at effector to target ratio E:T=2:1) was calculated based on the quantification of target cell death after 48 hours after subtracting the alloreactive background (assessed by the target cell death induced with the corresponding dextramer-negative T cell fraction) (see Methods). A particularly high specific lysis of the tumor cells was achieved with P1 and P2-specific T cells (35% and 44%, respectively), with a more moderate lysis (15%) with P6-specific T cells. The specific lysis was further increased when the target tumor cells were pulsed with the cognate epitope, reaching 55%, 80% and even 95% for P1, P2 and P6-specific T cells, respectively. Of note, epitope-specific T cell clones did not kill HLA-A2-positive human mammary epithelial cells (HMECs) used here as a negative, normal cell, control (FIG. 6C). These results were further validated using the 3D microscopy Nanolive technology, showing morphological signs of activation of specific T cells associated with killing of the majority of the tumor cells after 4.5 hours (E:T=10:1). Again, no cell death was observed when the specific T cells were co-cultured with HMECs and when MDA-MB-231 cells were co-cultured with non-specific T cells. Similar results were obtained using the TNBC HLA-A2-positive cell line HCC1599 as target. Altogether, these data show that the selected epitopes elicit high avidity CD8+ T cell clones that selectively recognize and kill HERV-expressing tumor cells.

### HERV-Specific T Cells are Present Among Tumor Infiltrating T Cells

In order to test our hypothesis that an adaptive immune response against HERVs may exist in cancer patients, we assessed by dextramer-staining the presence of HERV epitope-specific T cells among polyclonally expanded tumor infiltrating lymphocytes (TILs) from TNBC HLA-A2 patients (without any peptide-specific stimulation). HERV-specific TILs were observed for at least one epitope in 7 of the 11 analyzed tumor samples, with variations in terms of epitope specificity and frequency from one patient to another. P1, P4 and P6 were the most frequently recognized peptides, with a dextramer-based identification in 4/11, 4/11 and 5/11 cases, respectively.

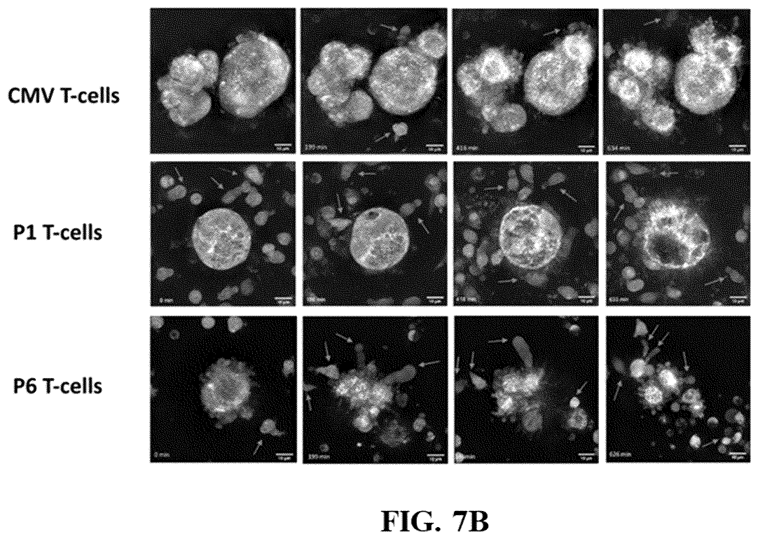

These results prompted us to investigate the potential link between the outcome of TNBC patients and the expression of the 18 CAHs containing these HLA-A2 epitopes. We established a score based on the mean expression of these 18 HERVs in HLA-A2 patients with basal breast cancer from TCGA cohort. Interestingly, HLA-A2-positive patients with a high or intermediate 18-HERVs score had a significantly better overall survival than those with a low score (P=0.0066) (FIG. 7A). This prognostic impact was not observed in the overall population.

Finally, we evaluated the antitumor activity of HERV-specific T cells against primary tumor cells by using organoids derived from the tumor of patient 8 (see Methods). RNAseq analysis confirmed the expression of the 18 epitopes-containing CAHs at early and late passage. Tumor organoids were co-cultured with P1, P6 or CMV-specific CD8+ T cell clones in a 3D microscopy Nanolive experiment (E:T=10:1). Whereas no activation of T cells was observed with CMV-specific T cells, P1 and P6-specific T cells exhibited signs of active proliferation associated with lysis of the organoids (FIG. 7B).

Altogether, these last results suggest that HERV-specific T cells are induced during tumor development and may participate in the antitumor immune response.

1-15. (canceled)

16. A method for identifying Human endogenous retroviruses (HERVs)-derived T cells epitopes associated with at least one cancer, wherein said method comprises the following steps:
(a) identifying HERVs associated with at least one cancer, and
(b) selecting T cell epitopes among the HERVs identified in the previous step, and
wherein said method further comprises at least one of the following steps:
(i) selecting HERVs associated with a cytotoxic T cells response among the cancer-associated HERVs identified in step (a), said step being between the step (a) and the step (b), and/or
(ii) assessing the expression at the protein or peptide level of the HERVs-derived T cells epitopes identified in step (b) in tumor samples, said step being after the step (b).

17. The method according to claim 16, wherein said method comprises the two steps (i) and (ii).

18. The method according to claim 16, wherein steps (a), (b), (i) and (ii) are in silico steps, or wherein steps (a), (b) and (i) are in silico steps and step (ii) is an in vitro step.

19. The method according to claim 16, wherein the step (a) comprises the step of comparing HERVs expression in tumor and in normal samples.

20. The method according to claim 16, wherein the step (b) comprises the step of aligning the sequences of the HERVs identified in the previous step with HERV proteins.

21. The method according to claim 20, wherein the step (b) further comprises the step of predicting the binding of the sequences sharing at least 70, 75, 80, 85, 90, 95, 96, 97, 98, 99% or more identity with HERV proteins to MHC class I molecules.

22. The method according to claim 16, wherein the association of the cancer-associated HERVs with a cytotoxic T cells response in the step (i) is assessed by the association of each HERV with at least one CD4 or CD8 T cell signature, the association of each HERV with a function signature being either interferon (IFN)-γ signature or cytolytic activity, and the absence of expression of each HERV in normal purified T or NK cells.

23. The method according to claim 22, wherein said association is assessed by a machine learning-based approach.

24. The method according to claim 16, wherein said method further comprises, after the step (b) or before the step (ii), a step of selecting epitopes among the most shared epitopes in the cancer-associated HERVs identified in step (a).

25. The method according to claim 16, wherein said method further comprises, after the step (b) or after the step (ii), a step of aligning the HERVs-derived T cell epitopes with human proteome.

26. A peptide comprising or consisting of an epitope identified by the method according to claim 16.

27. A peptide comprising or consisting of an epitope having a sequence selected from the group comprising or consisting of RMLTDLRAV (SEQ ID NO: 3), LMAQAITGV (SEQ ID NO: 11), VLQDFDQPI (SEQ ID NO: 13), MLLAALMIV (SEQ ID NO: 15) and YIDDILCAA (SEQ ID NO: 16).

28. An expression vector inducing expression of one or more peptide(s) according to claim 27.

29. A cytotoxic T-lymphocyte of a subject treated with one or more peptide(s) according to claim 27, or one or more expression vector(s) inducing expression of said one or more peptide(s).

30. An engineered T cell expressing a T-cell receptor recognizing a peptide according to claim 27.

31. A method for treating or preventing a cancer in a subject in need thereof, wherein said method comprises the administration of:
one or more peptide(s) according to claim 27,
one or more expression vector(s) inducing expression of said one or more peptide(s),
one or more cytotoxic T-lymphocyte(s) of a subject treated with said one or more peptide(s), or with said one or more expression vector(s), or
one or more engineered T cell(s) recognizing said one or more peptide(s).

32. The method according to claim 31, wherein said cancer is selected from the group comprising or consisting of breast cancer, ovarian cancer, melanoma, sarcoma, teratocarcinoma, bladder cancer, lung cancer, head and neck cancer, colorectal cancer, glioblastoma, leukemias, lymphomas and other solid tumors and hematological malignancies.

33. The method according to claim 32, wherein the breast cancer is triple negative breast cancer.

34. The method according to claim 32, wherein the lung cancer is non-small cell lung carcinoma or small cell lung carcinoma.
